(19) United States
(12) Patent Application Publication
(10) Pub. No.: US 2025/0281548 A1
(43) Pub. Date: Sep. 11, 2025

(54) ANTIMICROBIAL COMPOSITIONS COMPRISING EXTRACELLULAR VESICLES AND METHODS THEREOF

(71) Applicant: Washington State University, Pullman, WA (US)

(72) Inventors: Wenji Dong, Pullman, WA (US); Marwa Aly, Pullman, WA (US)

(21) Appl. No.: 19/073,347

(22) Filed: Mar. 7, 2025

Related U.S. Application Data
(60) Provisional application No. 63/562,657, filed on Mar. 7, 2024.

(51) Int. Cl. A61K 35/74; A61K 45/06; A61P 31/04; C12N 1/20

(52) U.S. Cl. CPC A61K 35/74; A61K 45/06; A61P 31/04; C12N 1/20

(57) ABSTRACT

The present disclosure provides compositions and methods comprising extracellular vesicles (EVs) derived from microbials and presenting a key alternative to currently available antimicrobials. The compositions and methods described herein utilize a mechanism of action that is currently unavailable with existing methods and can provide synergism to combat microbial infections. In particular, the compositions and methods of the present disclosure use EVs from certain stages of bacterial growth to accomplish their effectiveness, such as death EVs (D-EVs).

### CROSS-REFERENCE TO RELATED APPLICATIONS

This application claims the benefit under 35 U.S.C. § 119(e) of U.S. Provisional Application Ser. No. 63/562,657, filed on Mar. 7, 2024, the entire disclosures of which is incorporated herein by reference.

### BACKGROUND AND SUMMARY

Infections, including those caused by bacteria and by fungi, are an important public health concern. Although several antimicrobial therapies are currently available, the effectiveness of existing treatment options is a constant moving target for clinicians. For example, antibiotic-resistant infections possessing enhanced tolerance of high-dose antibiotic treatments are a critical threat due to their life-threatening and virulent nature.

Drug-resistant bacterial infections are a significant and growing challenge to global health. Several gram-positive and gram-negative bacterial strains, including Pseudomonas aeruginosa and methicillin-resistant Staphylococcus aureus (MRSA), have been identified as drug-resistant pathogens, or “superbugs.” In 2019, drug-resistant bacterial infections caused 1.27 million deaths, and this number is projected to increase to 10 million annual deaths worldwide by 2050. Existing antibiotics are often not effective enough at eradicating pathogenic bacteria.

Antibiotic resistance becomes especially problematic when the pathogenic cells grow as biofilms. Approximately 80% of drug-resistant bacterial infections in the human body are due to biofilms, and bacteria within these biofilms can increase their antibiotic resistance up to 1000-fold.

Both Gram-positive (+) and Gram-negative (−) bacterial strains can form multicellular community networks on wound beds; these are known as wound biofilms. Wound biofilms can comprise one or multiple types of bacterial species and variants, including MRSA and ESKAPE pathogens (Enterococcus faecium, Staphylococcus aureus, Klebsiella pneumoniae, Acinetobacter baumannii, Pseudomonas aeruginosa, and Enterobacter spp.). The biofilms prevent oxygen and drug penetration into the wound bed and evade host immune responses, thus increasing bacterial resistance to antibiotics and ultimately leading to treatment failure. Currently, most available antibiotics are incapable of eliminating drug-resistant wound biofilms. Therefore, there is an urgent need to develop more effective therapeutic approaches to combating wound biofilm infections. Moreover, it is highly desirable to provide therapies that can provide cross-species effectiveness and also utilize new mechanisms of action in treatment of microbials.

Accordingly, the present disclosure provides compositions and methods comprising extracellular vesicles (EVs) derived from microbials and presenting a key alternative to currently available antimicrobials. The compositions and methods described herein utilize a mechanism of action that is currently unavailable with existing methods and can provide synergism to combat microbial infections. In particular, the compositions and methods of the present disclosure use EVs from certain stages of bacterial growth to accomplish their effectiveness.

Development of biofilms generally involves multiple stages, including attachment, adaptation followed by exponential growth, maturation, stationary growth and dispersion leading to new biofilm formation. These stages can be influenced by the culture environment, with some stress or hostile culture conditions forcing the bacterial biofilm into a cell death/survival phase, in which bacterial populations can constantly switch among growth, survival, and death. Each of these developmental stages is tightly regulated by active cell-cell communication via various signaling mediators, including EVs to coordinate cellular processes.

EVs are membrane-bound nanovesicles with diameters of 30-400 nm that can be secreted by all types of cells. They transfer lipids, proteins, mRNAs, and microRNAs from parental cells to other cells, thus altering the target cells' behavior, making EVs important mediators of intercellular communication. Extracellular vesicle-based intercellular communication has been extensively studied in many biological and pathological processes, particularly in the context of cancer, but it is now recognized as a primordial feature of all living cells.

In recent years, it has been recognized that the secretion of EVs appears to be a conserved process in both Gram-negative and Gram-positive bacteria. For example, human pathogen Pseudomonas aeruginosa, an important Gram-negative bacterium and a major cause of infectious keratitis, uses EVs as a part of its signal trafficking system to mediate cell-cell communications within a bacterial community and coordinate group behaviors of the bacterial population. Other studies suggest that bacterial EVs are also involved in regulating essential cellular processes of bacterial life cycles, including cellular division, the formation and maintenance of biofilms, and the transferring of DNA to other bacteria sharing genes involved in antibiotic resistance.

In particular, the compositions and methods of the present disclosure utilize EVs captured from the planktonic and biofilm dispersal (death) phase of cells. For instance, the properties of these death EVs (“D-EVs”) are significantly influenced culture conditions. For example, conditions including inducing bacterial growth to the death phase under stress conditions and utilizing optimized nutrient profiles and environmental parameters that enhance D-EV bioactivity can provide enrichment of unique proteins and signaling molecules that are not present in naturally secreted EVs or EVs extracted under standard conditions.

As a result, the D-EVs contain specific molecules, such as dihydroxy acid dehydratase and heme acquisition proteins, that play pivotal roles in inducing bacterial programmed cell death (PCD). This distinct profile provides enriched EVs with proteins and signaling molecules unique to the death phase, such as those linked to ferroptosis and ROS production. As a result, the compositions and methods of the present disclosure containing D-EVs can be utilized as antimicrobials in numerous contexts, including treatment and prevention of biofilms, treatment of multidrug resistant pathogens, and treatment of cross-species pathogens by utilizing the mechanisms of action described herein.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTIONS OF THE DRAWINGS

The detailed description particularly refers to the accompanying figures in which:

FIG. 1 shows Kirby-Bauer disk diffusion susceptibility test of the sensitivity of P. aeruginosa PAO1 biofilms to 1-dose treatment with the D-EVs derived from PAO1, E. coli, and S. aureus, respectively. Tetracycline (10 mg/mL) and PBS buffer were used as a positive control and a negative control, respectively.

FIGS. 2A-2D show results of D-EV treatments of mouse wounds infected with PAO1. FIG. 2A is a photo shows the location of a wound created on the back of an animal. FIG. 2B displays images show 5-day healing processes of 24-h PAO1-infected wounds treated with PBS (control), or D-EVs (0.33 μg/μL). White circles indicate the original (zero-time) wound size of each condition. FIG. 2C shows cell viability analysis shows >3.5 log 10 reduction of bacterial cells 5 days after treatment of D-EVs (0.33 μg/μL) compared to that of PBS control (n=3). FIG. 2D shows the wound size was reduced >30% 5 days after single-dose treatment with D-EVs (0.33 μg/μL) compared to PBS control. Adding Fe³⁺ (25 μM) further reduced cell viability and wound size.

FIG. 3 illustrates extracellular vesicle extraction protocol in 6 major steps: (1) culturing PAO1 biofilm on filter membrane: P. aeruginosa PAO1 bacterial inoculum with 4.4×10⁵±7.3×10² CFU/cm² was used to seed a 47-mm sterilized membrane on sterilized TSA plates and incubated in the dark at 33° C. until biofilms reached either exponential growth or survival/death phases; (2) suspending and breaking up the biofilms to release EVs; (3) spinning down the bacterial cells; (4) filtering the supernatant through a 0.45 μm filter to remove cellular debris; (5) centrifuging at ultrahigh speed to isolate EVs from soluble proteins and other cellular molecules-after the EV pellet was resuspended in PBS buffer, the sample was filtered with a 0.22 μm syringe filter further to remove small cellular debris before the second ultrahigh-speed centrifugation to pellet down the target EVs; (6) suspending the EV pellet in PBS buffer and keeping the extracted EVs at −20° C. for further analysis.

FIGS. 4A-4D illustrate sizes and particle numbers of the purified D-EVs samples FIG. 4A illustrates a typical growth curve of P. aeruginosa PAO1 biofilm at 33° C., showing four stages of biofilm growth. Arrows indicate the stages where the biofilm samples were collected to extract G-EVs and D-EVs, respectively. Note: the scale of the x-axis is not linear. Data points indicate mean log CFU/cm², and error bars indicate standard deviations (n=4). FIG. 4B shows a fixed bed biofilm reactor filled with 4-mm borosilicate porous glass beads for biofilm growth. FIG. 4C shows TEM image and FIG. 4D depicts the size distributions of the D-EVs analyzed using a Zetasizer NS300 (Malvern Panalytical).

FIG. 5 shows quantitative fluorescence-based assay of EVs. EVs stained with Vybrant™ DiI Cell-Labeling Solution (red spots) were imaged under a confocal fluorescence microscope with an excitation/emission of 549/565 nm. The pictures from left to right show extracellular vesicle protein concentrations from low to high (20%=26.4±3 μg/ml, 40%=40.4±1 μg/ml, 80%=78.7±1 μg/ml, and 100%=96.4±4 μg/ml). The scale bar indicates 75 μm.

FIG. 6 plots the linear relationship of EV protein concentration (μg/ml) to EV particle number (×10³). Data points indicate the mean, and error bars indicate standard deviations (n=8).

FIG. 7 shows TEM images showing the sizes and shapes of the G-EVs and D-EVs from PAO1 biofilms. Samples are negatively stained with 2% uranyl acetate. Magnification power=25000×.

FIGS. 8A-8C exhibit a disc diffusion assay using both D-EVs and G-EVs to examine their functional effects on the growth behavior on bacterial biofilms. Kirby-Bauer disk diffusion susceptibility test of the effects of FIG. 8A, showing PAO1 D-EVs (1, 2, and 3 indicate 0.11, 0.22, and 0.33 μg/l, respectively) on PAO1 growth; FIG. 8B, showing the effect of intact D-EVs and lysed D-EVs on PAO1 growth; and FIG. 8C, showing D-EVs derived by MRSA (Staph) and E. coli PAO1 growth.

FIGS. 9A-9D show functional effect of G-EVs and D-EVs on the growth behavior of P. aeruginosa PAO1 biofilms. FIG. 9A shows growth of P. aeruginosa PAO1 biofilms starting from the initial phase after one-dose treatment with G-EVs (0.028 μg/μL) and D-EVs (0.33 μg/μL), respectively. The data points are the times at which the biomass of the biofilms under each condition was collected and analyzed. FIG. 9B shows growth of P. aeruginosa PAO1 biofilms (8-h) formed during the log growth phase after one-dose treatment with G-EVs (0.037 μg/μL) or D-EVs (0.33 μg/μL). Biomasses were collected and analyzed 24 hours after the biofilms were treated. FIG. 9C shows growth of P. aeruginosa PAO1 biofilms (24 h) developed during the stationary plateau phase after multidose treatment with D-EVs (0.33 μg/μL). Three doses of D-EVs were applied to the biofilms at 0, 24 and 48 hr. (indicated by J signs). The data points on each curve are the times at which the biomass of the biofilms under each condition was collected and analyzed. FIG. 9D shows growth of P. aeruginosa PAO1 biofilms (96-h) developed during the survival/death phase after one dose or 2 doses with a 12-h interval of D-EVs (0.33 μg/μL) or PBS. The mean value and error bar of each group of data are given in blue. For all data points, the biomasses of biofilms were collected and analyzed 24 hours after each treatment. P value <0.05 (*) and <0.01 (***).

FIGS. 10A-10F show TEM and confocal laser scanning microscopy (CLSM) of the interactions of the G-EVs/D-EVs and P. aeruginosa PAO1 bacterial cells. FIG. 10A is TEM images of the bacterial cell after incubation with G-EVs added to the cell culture. After 24 h, the cells were negatively stained with 2% uranyl acetate before TEM images were taken (magnification power=60000× and scale bar=200 nm). Blue arrows indicate either an individual EV or an EV that is interacting with the P. aeruginosa cell. FIG. 10B shows CLSM images of the bacterial cells that were incubated with DAPI-dye-labelled G-EVs. The images were taken with an excitation/emission of 359 nm/461 nm. Under this UV excitation, the recipient cells of DAPI-labeled G-EVs emit green fluorescence. The image on the left shows an overlay of the brightfield and fluorescent images, while the image on the right shows the fluorescent image only. The green fluorescence indicates the presence of the DAPI-labeled G-EVs. FIG. 10C shows TEM images of bacterial cells in the log phase after incubation with D-EVs. The cells were negatively stained with 2% uranyl acetate before TEM images were taken (magnification power=40000× and scale bar=100 nm). The image on the left shows a cell without D-EV treatment; double membrane layers are visible. The image on the right shows two cells treated with D-EVs. The membranes and cell walls of the cells have become damaged. The average sizes of P. aeruginosa PAO1 bacterial cells observed in images FIGS. 10A and 10C are between 0.6 and 1.2 um, consistent with earlier reports. FIG. 10D shows CLSM images of the bacterial cells that were positively stained with LIVE/DEAD BacLight Bacterial Viability Kits (excitation/emission: 480/500 nm for SYTO9 and 490/635 nm for propidium iodide). The image on the left shows the green, fluorescent live cells treated with PBS buffer; the image on the right shows the red dead cells (scale=75 μm) after incubation with D-EVs. The total corrected cell fluorescence (TCCF) of control cultures and cultures treated with SYTO™ 9 was calculated using the ImageJ method: TCCF=Integrated Intensity−(Area of selected cell×Mean fluorescence of background readings). SEM images were obtained after 24 hours of incubation in FIG. 10E (without D-EVs) and FIG. 10F (with D-EVs), with a scale bar of 3 μm.

FIG. 11 shows proteomic profiles of 79 surface and cytoplasmic proteins that are shared by both D-EVs and G-EVs with different abundances.

FIG. 12 shows inhibition effects of D-EVs on 96-h PAO1 biofilms. This graph on the left panel is the same as FIG. 9D, and it is reproduced here for comparison to FIG. 12, showing two doses of D-EVs at a protein concentration of 0.33 μg/μL applied to the biofilms with a 12-hour interval, which induced an inhibition of less than 1 log₁₀. FIG. 12, right panel, shows effects of 10 μM, 25 μM and 50 μM of ferric ions on 96-h biofilm. For all data points, biofilms were collected and analyzed 24 hours after each treatment. Ferric ions alone showed a very minimal effect on the biofilm growth. The mean value and error bar of each group of data are given in blue. P-value <0.05 (*).

FIG. 13 shows synergic effects of D-EVs/Fe³⁺ on 96-h P. aeruginosa PAO1 biofilm growth. For all data points, biomasses of biofilms were collected and analyzed 24 hours after each treatment. The mean value and error bar of each group of data are given in blue. P-value <0.05 (*), <0.03 (**) and <0.01 (***).

FIGS. 14A-14B show ROS levels of PAO1 biofilms. In FIG. 14A the ROS species were measured immediately after 1 hour of incubation of PAO1 bacteria in the lag phase with dye agents from Cellular ROS Assay Kit (Red) (ab186027), mixed with PBS buffer, tetracycline (10 mg/ml), or D-EVs (97.9 μg/ml). Increase in fluorescence intensity (λₑₓ/λₑₘ: 520/605 nm) was correlated with increase of overall ROS level in each sample. FIG. 14B monitors the H₂O₂ level in PAO1 biofilms. 24 hr. grown biofilms were treated with PBS, Tetracycline (0.5 mg/mL), PAO1-D-EVs (115.75 μg/ml), PAO1 D-EVs+Fer-1 (10 μM and 10 mM) or PAO1 D-EVs+Def (10 μM and 10 mM), and H₂O₂ levels were immediately monitored by measuring the fluorescence intensity (excitation/emission: 490/525 nm) with a plate reader using cell based assay kit (ab138874, Abcam). The hydrogen peroxide level was correlated to the fluorescence intensity in all samples. Data points represent the mean and the error bars indicate the standard deviation (n=5)

FIGS. 15A-15D show a cytotoxicity test of D-EVs to human mesenchymal stem cell culture. FIG. 15A shows changes in hMSC growth in the absence/presence of D-EVs (0.33 μg/μl) monitored for two days. Light microscopy images show no change in the cellular morphologies of human mesenchymal stem cells (hMSC) after two days of growth in FIG. 15A (the absence of D-EVs from PAO1) and in FIG. 15B (the presence of D-EVs (0.33 μg/μl) from PAO1). At each time point, cells were stained with Cell Proliferation Reagent WST-1 and incubated for four hours. The absorbance was measured at 450 nm to monitor the replication of their genomic DNA.

FIG. 15C shows cell proliferation (growth) of hMSC monitored via the absorbance of the same hMSC samples as FIG. 15A and FIG. 15B. The hMSC growth was not affected by the presence of the D-EVs. The absorbance was measured at 450 nm to monitor the cellular replications. FIG. 15D is a quantification of the LPS contents of G-EVs, D-EVs and PAO1 cells determined using a Pierce Chromogenic Quant Kit (A395526S, ThermoFisher).

FIGS. 16A-16H summarize the EV bacterial protocol adapted for a fungal pathogen, C. auris. FIG. 16A depicts a growth of inhibition of growth of fresh planktonic Candida auris cells (OD₆₀₀=0.5) in yeast peptone dextrose (YPD) broth medium by one-dose treatment with D-EVs extracted from either PAO1 (0.25 μg/μl) or E. coli (0.15 μg/μl) (n=3). Fungal cell growth was monitored by measuring the OD₆₀₀ of the cell culture. Bacterial D-EVs and H₂O₂ (30%) effectively inhibited fungal growth. FIG. 16B shows the inhibition of C. auris biofilms by one-dose treatment with D-EVs extracted from PAO1 (0.25 μg/μl) (n=2), and FIG. 16B shows the inhibition of C. auris biofilms (24 hr. of growth) by one-dose treatment with D-EVs extracted from PAO1 (0.25 μg/μl) (n=4). FIG. 16D illustrates the overall cellular levels of ROS radicals (including ·O₂⁻ and ·OH) of C. auris in YPD broth culture in response to bacterial D-EV treatments. After 24 hr. of growth, C. auris cell culture (OD₆₀₀=0.5) was mixed with a Cellular ROS Assay Kit (ab186027) and further incubated for 1 hr. Then, one dose of D-EVs extracted from either PAO1 (0.25 μg/μl) or E. coli (0.15 μg/μl) was added, followed by immediate monitoring of changes in fluorescence intensity (n=4). No change in ROS production was observed when the samples were treated with PBS, 2 mg/mL of ertapenem (ETP) and 2 mg/mL of tetracycline (TETRA). H₂O₂ (20%) was used as a positive control to stimulate C. auris to produce ·OH species. FIGS. 16E-16H show SEM images of C. auris cells at 0 hr., 6 hr., 12 hr. and 24 hr. respectively, after one-dose treatment with PAO1-derived D-EVs (0.25 μg/μl), which clearly show the processes of fungal cell destruction.

FIG. 17 depicts a schematic summarizing the protocol for C. auris biofilm preparation and treatment with bacterial D-EVs.

FIG. 18 is a graph of changes in levels of intracellular H₂O₂ in 24-h C. auris biofilm after treatment with PAO1 D-EVs (0.10 μg/μL) and echinocandins (1.0 μg/μL). H₂O₂ levels were monitored via fluorescence intensity (ex/em: 490 nm/525 nm) using an H₂O₂ assay kit (ab138874, Abcam). No H₂O₂ production was observed for treatment with antifungal echinocandins because the drug targets cell wall synthesis rather than H₂O₂ pathways.

FIG. 19 illustrates a porcine ex vivo model for testing the efficacy of bacterial D-EVs.

FIG. 20 depicts a graph of P. aeruginosa PAO1 biofilm growth following treatment with D-EVs±iron. Growth of P. aeruginosa PAO1 biofilms at 2 h, 8 h, 24 h, 48 h, and 96 hr. were calculated as Log₁₀ CFU/cm² following 24 hr. of exposure to the respective treatment. Cells were treated with D-EVs at a concentration of 9.44×10¹¹±3.6×10¹⁰ particle/mL±50 μl Fe³⁺. The data points represent the mean values of three replicates.

FIG. 21 shows a graph monitoring the total free iron levels in P. aeruginosa PAO1 biofilms. P. aeruginosa PAO1 biofilms were grown on TSA plates and incubated at 33° C. 24 hr. biofilms were incubated with the dye agent for 1 h, followed by treatment with PBS, DMSO, Tetracycline (0.5 mg/mL), PAO1-D-EVs (115.75 μg/ml), PAO1 D-EVs+Fer-1 (10 μM), or PAO1 D-EVs+Def (10 μM). The iron inducer and iron probe were added and free total iron levels were monitored by measuring the absorbance at 593 nm with a plate reader. Free iron concentration was calculated based on a standard curve, where the data points were measured for 3 replicates.

FIG. 22 depicts the quantification of Reactive oxygen species (ROS) levels in P. aeruginosa PAO1 biofilms. P. aeruginosa PAO1 biofilms were grown on TSA plates and incubated at 33° C. After 24 hours, the biofilms were treated with PBS, Tetracycline (0.5 mg/mL), PAO1-D-EVs (115.75 μg/ml), PAO1 D-EVs+Fer-1 (10 μM) or PAO1 D-EVs+Def (10 μM). The ROS levels were immediately measured thereafter by monitoring the fluorescence intensity (excitation/emission: 520/605 nm). The ROS level was correlated to the fluorescence intensity for all samples. Data points represent the mean with standard deviation (n=3).

FIG. 23 is a graph quantifying the reactive oxygen species concentration in P. aeruginosa PAO1 biofilms. P. aeruginosa PAO1 biofilms were grown on TSA plates and incubated at 33° C. After 24 hr. the biofilms were treated with PBS, Tetracycline (0.5 mg/mL), PAO1-D-EVs (115.75 μg/ml), PAO1 D-EVs+Fer-1 (10 μM) or PAO1 D-EVs+Def (10 μM), and ROS levels were immediately monitored by measuring the fluorescence intensity (excitation/emission: 520/605 nm) with a plate reader. The ROS level was correlated to the fluorescence intensity in all samples. Data points represent the mean and the error bars indicate the standard deviation (n=3).

FIG. 24 shows the hydrogen peroxide levels in P. aeruginosa PAO1 biofilms. P. aeruginosa PAO1 biofilms were grown on TSA plates and incubated at 33° C. After 24 hr., the biofilms were treated with PBS, Tetracycline (0.5 mg/mL), PAO1-D-EVs (115.75 μg/ml), PAO1 D-EVs+Fer-1 (10 μM) or PAO1 D-EVs+Def (10 PM), and hydrogen peroxide levels were immediately monitored by measuring the fluorescence intensity (excitation/emission: 490/525 nm) with a plate reader. The hydrogen peroxide levels were correlated to the fluorescence intensity in all samples. Data points represent the mean and the error bars indicate the standard deviation (n=3).

FIG. 25 shows E. coli K12, ΔkatE, ΔkatG, and ΔkatEG, biofilms treated with D-EVs. Growth of 24 hr. biofilms were calculated as Log₁₀ CFU/μL after 24 hr. of exposure to the treatment. Cells were treated with D-EVs (9.44×10¹¹±3.6×10¹⁰ particle/mL). The data points represent the mean values of two replicates and the mean values±SD.

FIG. 26 depicts a graph of Lipid hydroperoxide levels in P. aeruginosa PAO1 biofilms. P. aeruginosa PAO1 biofilms were grown on TSA plates incubated at 33° C. The biofilms were treated for 24 hr with one of: PBS, Tetracycline (0.5 mg/mL), PAO1-D-EVs (115.75 μg/ml), PAO1 D-EVs+Fer-1 (10 μM) or PAO1 D-EVs+Def (10 μM). Lipid hydroperoxides were extracted in chloroform then applied to the colorimetric kit. Data points represent the mean and the error bars indicate the standard deviation (n=3).

FIG. 27 shows the quantification of lactate dehydrogenase activity in P. aeruginosa PAO1 biofilms. P. aeruginosa PAO1 biofilms were grown on TSA plates and incubated at 33° C. 24 hr. biofilms were treated for 24 hr. with PBS, Tetracycline (0.5 mg/mL), PAO1-D-EVs (115.75 μg/ml), PAO1 D-EVs+Fer-1 (10 μM) or PAO1 D-EVs+Def (10 μM). Lactate dehydrogenase was extracted then applied to the kit. Data points represent the mean and the error bars indicate the standard deviation (n=2).

FIG. 28 depicts quantification of superoxide levels in P. aeruginosa PAO1 biofilms. P. aeruginosa PAO1 biofilms were grown on TSA plates and incubated at 33° C. 24 hr. biofilms were treated with PBS, Tetracycline (0.5 mg/mL), PAO1-D-EVs (115.75 μg/ml), PAO1 D-EVs+Fer-1 (10 μM) or PAO1 D-EVs+Def (10 PM), and superoxide levels were immediately monitored by measuring the fluorescence intensity (excitation/emission: 488/520 nm) with a plate reader. The superoxide level was correlated to the fluorescence intensity in all samples. Data points represent the mean and the error bars indicate the standard deviation (n=3).

### DETAILED DESCRIPTION

Various embodiments of the invention are described herein as follows.

In an illustrative aspect, a composition comprising one or more death extracellular vesicles (D-EVs) is provided.

The D-EVs as described herein arise under specific, human-controlled death-phase conditions such as bacterial starvation, stress, and the like. As described herein, proteomic data demonstrate that D-EVs have a unique molecular signature that are distinct from normal, non-death extracellular vesicles.

The D-EVs include proteins that are absent in the non-death EVs. For instance, D-EVs can contain iron-regulating enzymes to facilitate ferroptosis and oxidative stress, redox-cycle regulators to promote ROS accumulation inside microbial cells, and AHL acylases to disrupt quorum sensing and prevent formation of biofilms. Thus, proteomic analysis confirms that D-EVs have a significantly altered protein signatures compared to naturally secreted extracellular vesicles, making them functionally distinct.

Various D-EV specific proteins include those in one or more of the following categories and fully described in Table 5 herein:

Iron Acquisition Systems: D-EVs are enriched in iron-scavenging and uptake proteins that growth phase EVs (G-EVs). For example, D-EVs contain outer-membrane receptors for siderophores and heme that are absent in G-EVs, such as a heme acquisition protein (HasA) and multiple TonB-dependent ferric siderophore receptors (for pyochelin, enterobactin, etc.). These proteins are normally be expressed during iron starvation. Their presence suggests D-EVs can sequester or deliver iron-binding factors, a feature not seen in normal EVs from bacteria under nutrient-replete conditions.

ROS Generation & Redox Modulators: Several D-EV-exclusive proteins are associated with production or regulation of reactive oxygen species (ROS). Notably, a soluble pyridine nucleotide transhydrogenase can be present to alter the NADH/NADPH balance, potentially reducing antioxidant capacity in target cells by depleting NADPH. A quinone oxidoreductase and other dehydrogenases are also unique to D-EVs, and may promote H₂O₂ generation. Their inclusion in D-EVs indicates the vesicles can induce oxidative stress in microbes.

Quorum-Sensing Quenchers: D-EVs can include enzymes that interfere with cell-cell signaling. In particular, an N-acyl-homoserine lactone (AHL) acylase (quorum-quenching enzyme) was identified in D-EVs. This enzyme degrades bacterial quorum-sensing signals (AHLs). Inclusion of an AHL-degrading enzyme permits D-EVs to disrupt quorum sensing in other bacteria,

Structural & Regulatory Proteins: D-EVs can also unexpectedly contain structural and regulatory proteins. For example, flagellin fragments and flagellar structural proteins (L-ring and P-ring components) were identified, likely due to cell lysis or membrane blebbing during the death phase. Further, outer membrane porins (OprD family) and several transcriptional regulators (TetR-family, two-component response regulators) were also uniquely present in D-EVs.

Further, the addition of exogenous iron during D-EV induction causes distinct shifts in vesicle protein content. Various observations include those in the following categories and fully described in Table 6 herein:

Suppression of Iron-Scavenging Proteins: D-EVs produced with iron supplementation show marked reduction or absence of the siderophore receptors that dominate normal D-EVs. For example, iron-starved D-EVs can include abundant HasA and ferric-siderophore receptors, whereas in iron-supplemented D-EVs these proteins were either greatly diminished or not detected. This indicates that those unique D-EV components are an induced response to iron starvation. In contrast, D-EVs formed without supplemental iron are loaded with iron-scavenging machinery.

Enhanced Oxidative Stress Factors: Providing excess iron during vesicle production may elevate certain oxidative stress-related proteins in D-EVs. Data suggest that antioxidant enzymes and redox regulators remain in D-EVs even with iron present, and some (e.g. NADH dehydrogenases or peroxidases) potentially become more prominent. For instance, pyridine nucleotide transhydrogenase (a protein that can drive ROS generation in target cells) was still present at high levels in iron-supplemented D-EVs (similar to non-supplemented D-EVs). Although iron supplementation suppresses the iron-scavenging elements of D-EVs, it does not eliminate the ROS-inducing and other stress components. Some stress proteins may even be up-regulated in response to iron overload (e.g. induction of ferric iron storage or detox enzymes).

Overall Proteome Shift: In sum, iron-supplemented D-EVs have a slightly different proteomic fingerprint than regular D-EVs, but both remain clearly distinct from any naturally occurring EVs. As shown in Table 6 herein, the unique composition of D-EVs is retained (e.g., many proteins such as quorum quenchers, redox enzymes, and the like) are still present with iron supplementation but emphasis can shift away from iron-acquisition and towards other stress-related functions.

Normal, non-death EVs play a role in cell signaling but do not actively induce cell death. In contrast, D-EVs can trigger a ferroptosis-like mechanism, characterized by elevated ROS production (e.g., hydroxyl radicals, superoxides, and lipid hydroperoxides, disruption of iron metabolism within bacterial biofilms, leading to oxidative damage, and increased susceptibility to oxidative stress, especially in bacteria lacking antioxidant enzymes (e.g., catalase-peroxidase mutants).

Further, as described herein, the D-EVs exhibit novel therapeutic functionalities such as triggering ROS-based ferroptosis in other microbes and inhibiting biofilm growth cross-species. D-EVs have been shown to inhibit biofilm formation and eliminate up to 99.99% of mature biofilms, including those of antibiotic-resistant bacteria (e.g., P. aeruginosa, MRSA, and ESKAPEE pathogens). Unlike conventional antibiotics, D-EVs can target microbial redox balance and induce oxidative stress-driven cell death, which reduces the likelihood of resistance development. Such effects of D-EVs can include:

Iron Uptake Overload & Ferroptosis-Like Cell Death: D-EVs can drive excess iron into target bacteria, thus instituting a lethal Fenton reaction inside the cells. Many D-EV-specific proteins (see Table 5) are involved in iron uptake and metabolism. When D-EVs encounter a target microbe, these proteins likely stimulate the microbe to import iron or supply it with iron-loaded compounds. In the presence of the hydrogen peroxide from the microbe (or other basal ROS), the excess iron catalyzes production of highly destructive hydroxyl radicals (OH·) via Fenton chemistry. This mechanism is analogous to ferroptosis, an iron-dependent programmed cell death originally characterized in eukaryotes. This mechanism can arise from the unique composition of D-EVs and adding Fe³⁺ can synergistically increase the antimicrobial efficacy of D-EVs, causing >3-log biofilm cell death in otherwise highly resilient 96-hours.

Induction of Oxidative Stress: D-EVs can also include proteins that directly disrupt redox homeostasis in target cells. For instance, the presence of transhydrogenase and oxidoreductases in D-EVs suggests that fusion of the D-EVs can alter the intracellular NADH/NADPH balance and generate ROS. For example, a pyridine nucleotide transhydrogenase can siphon reducing power away from glutathione regeneration, tilting the glutathione redox ratio (GSH/GSSG) toward an oxidized. Similarly, any delivered phenazine/pyocyanin redox cycling factors would increase superoxide and peroxide production. The net result is heightened oxidative stress inside the target microbe, making it more susceptible to damage or death.

Quorum Sensing Interference: D-EVs also exhibit a quorum-quenching effect by including AHL acylase enzymes. By degrading N-acyl-homoserine lactones, D-EVs can disrupt the communication signals that bacteria use to coordinate group behaviors (e.g., biofilm formation and virulence). Quorum sensing inhibition weakens target biofilms.

In an embodiment, the one or more D-EVs comprise one or more proteins selected from the group consisting of a redox cycle-regulating protein, an iron-acquisition protein, a quorum sensing (QS) protein, and any combination thereof.

In an embodiment, the one or more D-EVs comprise a redox cycle-regulating protein. In an embodiment, the one or more D-EVs comprise an iron-acquisition protein. In an embodiment, the one or more D-EVs comprise a quorum sensing (QS) protein.

In an embodiment, the one or more D-EVs comprise one or more proteins selected from Table 5. In an embodiment, the one or more D-EVs comprise one or more proteins selected from Table 6. In an embodiment, the one or more D-EVs comprise one or more proteins selected from Table 5 and Table 6.

In an embodiment, the one or more D-EVs is isolated from a microorganism.

In an embodiment, the microorganism is an ESKAPEE pathogen. In an embodiment, the ESKAPEE pathogen is Enterococcus. In an embodiment, the ESKAPEE pathogen is Enterococcus spp. In an embodiment, the Enterococcus is Enterococcus faecium.

In an embodiment, the ESKAPEE pathogen is Staphylococcus aureus. In an embodiment, the S. aureus is methicillin-resistant (MRSA).

In an embodiment, the ESKAPEE pathogen is Klebsiella pneumoniae. In an embodiment, the ESKAPEE pathogen is Acinetobacter baumanii.

In an embodiment, the ESKAPEE pathogen is Pseudomonas aeruginosa. In an embodiment, the P. aeruginosa is PAO1.

In an embodiment, the ESKAPEE pathogen is Enterobacter. In an embodiment, the ESKAPEE pathogen is Enterobacter spp. In an embodiment, the ESKAPEE pathogen is Escherichia coli.

In an embodiment, the microorganism is a bacterium. In an embodiment, the bacterium is an antibiotic-resistant bacterium. In an embodiment, the bacterium is a Gram-negative bacterium. In an embodiment, the bacterium is a Gram-positive bacterium. In an embodiment, the bacterium is an ESKAPEE pathogen.

In an embodiment, the microorganism is a fungus. In an embodiment, the fungus is Candida. In an embodiment, the fungus is Candida spp. In an embodiment, the fungus is Candida auris.

In an embodiment, the one or more D-EVs is isolated from a microorganism under a stress-induced condition. In an embodiment, the composition is an antimicrobial composition.

In an illustrative aspect, a pharmaceutical composition comprising i) one or more death extracellular vesicles (D-EVs) and ii) a pharmaceutically acceptable carrier is provided.

In an embodiment, the composition is any one of the compositions as described herein.

In an embodiment, the pharmaceutical composition further comprises a metal ion. In an embodiment, the metal is an iron. In an embodiment, the iron is Fe²⁺. In an embodiment, the iron is Fe³⁺. In an embodiment, the pharmaceutical composition further comprises an iron chelator.

In an embodiment, the pharmaceutical composition further comprises a second therapeutic agent. In an embodiment, the second therapeutic agent is an antibiotic. In an embodiment, the second therapeutic agent is a fungicide.

In an embodiment, the pharmaceutical composition is a topical formulation. In an embodiment, the pharmaceutical composition is a parenteral formulation. In an embodiment, the pharmaceutical composition is an aerosolized formulation.

In an illustrative aspect, a method of treating an infection in a patient is provided. The method comprises a step of administering a therapeutically effective amount of a pharmaceutical composition comprising i) one or more death extracellular vesicles (D-EVs) and ii) a pharmaceutically acceptable carrier to the patient, In an embodiment, the pharmaceutical composition provides treatment of the infection.

In an embodiment, the pharmaceutical composition is any one of the pharmaceutical compositions as described herein.

In an embodiment, the infection is caused by a microbe. In an embodiment, the microbe is an ESKAPEE pathogen.

In an embodiment, the ESKAPEE pathogen is Enterococcus. In an embodiment, the ESKAPEE pathogen is Enterococcus spp. In an embodiment, the Enterococcus is Enterococcus faecium.

In an embodiment, the ESKAPEE pathogen is Staphylococcus aureus. In an embodiment, the S. aureus is methicillin-resistant (MRSA).

In an embodiment, the ESKAPEE pathogen is Klebsiella pneumoniae. In an embodiment, the ESKAPEE pathogen is Acinetobacter baumanii.

In an embodiment, the ESKAPEE pathogen is Pseudomonas aeruginosa. In an embodiment, the P. aeruginosa is PAO1.

In an embodiment, the ESKAPEE pathogen is Enterobacter. In an embodiment, the ESKAPEE pathogen is Enterobacter spp. In an embodiment, the ESKAPEE pathogen is Escherichia coli.

In an embodiment, the microbe is a bacterium. In an embodiment, the bacterium is an antibiotic-resistant bacterium. In an embodiment, the bacterium is a Gram-negative bacterium. In an embodiment, the bacterium is a Gram-positive bacterium. In an embodiment, the bacterium is an ESKAPEE pathogen.

In an embodiment, the microbe is a fungus. In an embodiment, the fungus is Candida. In an embodiment, the fungus is Candida spp. In an embodiment, the fungus is Candida auris.

In an embodiment, the treatment of the infection comprises growth inhibition of the microbe. In an embodiment, the treatment of the infection comprises reduction of growth of a biofilm comprising the microbe. In an embodiment, the treatment of the infection comprises reduction of size of a biofilm comprising the microbe. In an embodiment, the treatment of the infection comprises reduction of adhesion of the microbe.

In an embodiment, the treatment of the infection comprises ROS-based ferroptosis of the microbe. In an embodiment, the treatment of the infection comprises an increase of ROS accumulation in the microbe. In an embodiment, the treatment of the infection comprises an increase in iron accumulation by the microbe. In an embodiment, the treatment of the infection comprises disruption of iron metabolism of the microbe.

In an embodiment, the treatment of the infection comprises increased susceptibility of the microbe to oxidative stress. In an embodiment, the treatment of the infection comprises induction of oxidative stress-induced cell death of the microbe. In an embodiment, the treatment of the infection comprises disruption of quorum sensing of the microbe.

In an embodiment, the method further comprises administration of a metal ion to the patient. In an embodiment, the metal is an iron. In an embodiment, the iron is Fe²⁺. In an embodiment, the iron is Fe³⁺. In an embodiment, the method further comprises administration of an iron chelator to the patient.

In an embodiment, the method further comprises administration of a second therapeutic agent to the patient. In an embodiment, the second therapeutic agent is an antibiotic. In an embodiment, the second therapeutic agent is a fungicide. In an embodiment, the second therapeutic agent is a debridement.

In an embodiment, the administration is a topical administration. In an embodiment, the administration is a parenteral administration. In an embodiment, the administration is an aerosolized administration.

In an embodiment, the infection is a wound. In an embodiment, the wound is a chronic wound. In an embodiment, the wound is a surgical wound. In an embodiment, the wound is an ulcer. In an embodiment, the wound is a wound bed. In an embodiment, the wound is an organ transplant wound.

In an embodiment, the infection is a burn.

In an embodiment, the infection is a pulmonary infection. In an embodiment, the pulmonary infection is cystic fibrosis. In an embodiment, the pulmonary infection is pneumonia. In an embodiment, the pulmonary infection is ventilator-associated pneumonia.

In an illustrative aspect, a method of collecting one or more extracellular vesicles (EVs) from a composition comprising one or more microorganisms is provided. The method comprises the steps of (a) culturing the one or more microorganisms, wherein the culturing comprises secretion of one or more EVs from the microorganisms; (b) separating the EVs from the composition; and (c) collecting the EVs separated in step (b).

In an embodiment, the composition comprises a microbial culture. In an embodiment, the composition comprises a biofilm. In an embodiment, the composition comprises a planktonic culture. Generally, a planktonic culture refers to a plurality of free-floating microorganisms that are not attached to surfaces or to each other.

In an embodiment, the culturing comprises induction of a stress condition to the one or more microorganisms. In an embodiment, the stress condition comprises a temperature stress condition. In an embodiment, the temperature stress condition is a heat stress. In an embodiment, the temperature stress condition is a decreased temperature.

In an embodiment, the stress condition comprises a nutrient starvation. In an embodiment, the nutrient starvation comprises a reduction of carbon in the composition.

In an embodiment, the stress condition comprises an oxidative stress. In an embodiment, the oxidative stress comprises a reactive oxygen species. In an embodiment, the oxidative stress comprises a reactive nitrogen species.

In an embodiment, the stress condition comprises decreasing pH of the composition to below a pH of 7. In an embodiment, the stress condition comprises increasing pH of the composition to above a pH of 7. In an embodiment, the stress condition comprises increasing osmotic pressure of the composition to a pressure of 1,700 mosmol or higher. In an embodiment, the stress condition comprises increasing the salinity of the composition. In an embodiment, the stress condition comprises applying UV stress to the composition.

In an embodiment, the stress condition comprises introducing a chemical to the composition. In an embodiment, the chemical is a fungicide. In an embodiment, the chemical is an antibiotic. In an embodiment, the chemical is an antimicrobial peptide.

In an embodiment, the chemical is a metal. In an embodiment, the metal is aluminum. In an embodiment, the metal is copper. In an embodiment, the metal is silver.

In an embodiment, the metal is an iron. In an embodiment, the iron is Fe²⁺. In an embodiment, the iron is Fe³⁺.

In an embodiment, the stress condition comprises deprivation of oxygen to the composition.

In an embodiment, the stress condition is selected from the group consisting of a temperature stress condition, a nutrient starvation, an oxidative stress, decreasing the pH of the composition to below a pH of 7, increasing the pH of the composition to above a pH of 7, increasing the osmotic pressure of the composition, increasing the salinity of the composition, applying UV stress to the composition, introducing a chemical to the composition, deprivation of oxygen to the composition, or combinations thereof.

In an embodiment, the method further comprises extracting the EVs from the composition. In an embodiment, the separating comprises centrifugation. In an embodiment, the separating comprises a chemical gradient. In an embodiment, the method further comprises concentrating the EVs.

In an embodiment, the one or more EVs comprise a death EV (D-EV). Generally, a death EV (D-EV) refers to one or more EVs that are obtained during the dispersal (death) phase of cells. In an embodiment, the one or more EVs consists essentially of a death EV (D-EV). In an embodiment, the one or more EVs consists of a death EV (D-EV).

In an embodiment, the one or more microorganisms comprise a consortium of microorganisms. In an embodiment, the consortium of microorganisms comprises at least one species of an ESKAPEE pathogen. Generally, ESKAPEE pathogens refer to a group of highly antibiotic-resistant bacteria including Enterococcus faecium, Staphylococcus aureus, Klebsiella pneumoniae, Acinetobacter baumannii, Pseudomonas aeruginosa, Enterobacter spp. and Escherichia coli. In an embodiment, the ESKAPEE pathogen is Enterococcus. In an embodiment, the ESKAPEE pathogen is Enterococcus spp. In an embodiment, the Enterococcus is Enterococcus faecium. In an embodiment, the ESKAPEE pathogen is Staphylococcus aureus. In an embodiment, the S. aureus is methicillin-resistant (MRSA).

In an embodiment, the ESKAPEE pathogen is Klebsiella pneumoniae. In an embodiment, the ESKAPEE pathogen is Acinetobacter baumanii. In an embodiment, the ESKAPEE pathogen is Pseudomonas aeruginosa. In an embodiment, the P. aeruginosa is PAO1.

In an embodiment, the ESKAPEE pathogen is Enterobacter. In an embodiment, the ESKAPEE pathogen is Enterobacter spp. In an embodiment, the ESKAPEE pathogen is Escherichia coli.

In an embodiment, the one or more microorganisms is a bacterium. In an embodiment, the bacterium is an antibiotic-resistant bacterium. In an embodiment, the bacterium is a Gram-negative bacterium. In an embodiment, the bacterium is a Gram-positive bacterium.

In an embodiment, the bacterium is an ESKAPEE pathogen. In an embodiment, the ESKAPEE pathogen is Enterococcus. In an embodiment, the ESKAPEE pathogen is Enterococcus spp. In an embodiment, the Enterococcus is Enterococcus faecium. In an embodiment, the ESKAPEE pathogen is Staphylococcus aureus. In an embodiment, the S. aureus is methicillin-resistant (MRSA).

In an embodiment, the ESKAPEE pathogen is Klebsiella pneumoniae. In an embodiment, the ESKAPEE pathogen is Acinetobacter baumanii. In an embodiment, the ESKAPEE pathogen is Pseudomonas aeruginosa. In an embodiment, the P. aeruginosa is PAO1.

In an embodiment, the ESKAPEE pathogen is Enterobacter. In an embodiment, the ESKAPEE pathogen is Enterobacter spp. In an embodiment, the ESKAPEE pathogen is Escherichia coli.

In an embodiment, the one or more microorganisms is a fungus. In an embodiment, the fungus is Candida. In an embodiment, the fungus is Candida spp. In an embodiment, the fungus is Candida auris.

In an embodiment, the one or more microorganisms is an engineered microorganism. In an embodiment, the engineered microorganism is an engineered bacterium. In an embodiment, the engineered microorganism is an engineered fungus.

In an embodiment, the separating is selected from a group consisting of centrifugation, a chemical gradient, filtration, or a combination thereof.

In an embodiment, the collected EVs are subsequently lyophilized. In an embodiment, the collected EVs are subsequently encapsulated.

In an illustrative aspect, an extracellular vesicle (EV) obtained by the method of collecting is provided. The EV, or a plurality of EVs, can comprise any of the features described according to the method of collection. In an embodiment, the EV is lyophilized. In an embodiment, the EV is encapsulated.

In an illustrative aspect, a method of inhibiting a biofilm is provided. The method comprises a step of applying an effective amount of one or more extracellular vesicles (EVs) collected from one or more microorganisms, where the EVs provide inhibition of the biofilm.

In an embodiment, the EVs are obtained by the method of any of the of collecting embodiments as described herein. In an embodiment, the inhibition comprises a reduction of growth of the biofilm. In an embodiment, the inhibition comprises a reduction in size of the biofilm.

In an embodiment, the one or more microorganisms comprise a consortium of microorganisms. In an embodiment, the consortium of microorganisms comprises at least one species of an ESKAPEE pathogen. In an embodiment, the ESKAPEE pathogen is Enterococcus. In an embodiment, the ESKAPEE pathogen is Enterococcus spp. In an embodiment, the Enterococcus is Enterococcus faecium. In an embodiment, the ESKAPEE pathogen is Staphylococcus aureus. In an embodiment, the S. aureus is methicillin-resistant (MRSA).

In an embodiment, the ESKAPEE pathogen is Klebsiella pneumoniae. In an embodiment, the ESKAPEE pathogen is Acinetobacter baumanii. In an embodiment, the ESKAPEE pathogen is Pseudomonas aeruginosa. In an embodiment, the P. aeruginosa is PAO1.

In an embodiment, the ESKAPEE pathogen is Enterobacter. In an embodiment, the ESKAPEE pathogen is Enterobacter spp. In an embodiment, the ESKAPEE pathogen is Escherichia coli.

In an embodiment, the one or more microorganisms is a bacterium. In an embodiment, the bacterium is an antibiotic-resistant bacterium. In an embodiment, the bacterium is a Gram-negative bacterium. In an embodiment, the bacterium is a Gram-positive bacterium.

In an embodiment, the bacterium is an ESKAPEE pathogen. In an embodiment, the ESKAPEE pathogen is Enterococcus. In an embodiment, the ESKAPEE pathogen is Enterococcus spp. In an embodiment, the Enterococcus is Enterococcus faecium. In an embodiment, the ESKAPEE pathogen is Staphylococcus aureus. In an embodiment, the S. aureus is methicillin-resistant (MRSA).

In an embodiment, the ESKAPEE pathogen is Klebsiella pneumoniae. In an embodiment, the ESKAPEE pathogen is Acinetobacter baumanii. In an embodiment, the ESKAPEE pathogen is Pseudomonas aeruginosa. In an embodiment, the P. aeruginosa is PAO1.

In an embodiment, the ESKAPEE pathogen is Enterobacter. In an embodiment, the ESKAPEE pathogen is Enterobacter spp. In an embodiment, the ESKAPEE pathogen is Escherichia coli.

In an embodiment, the one or more microorganisms is a fungus. In an embodiment, the fungus is Candida. In an embodiment, the fungus is Candida spp. In an embodiment, the fungus is Candida auris.

In an embodiment, the one or more microorganisms is an engineered microorganism. In an embodiment, the engineered microorganism is an engineered bacterium. In an embodiment, the engineered microorganism is an engineered fungus.

In an embodiment, the biofilm comprises one or more microbes. In an embodiment, the EVs provide inhibition of the one or more microbes of the biofilm. In an embodiment, the EVs provide inhibition of the one or more microbes of the biofilm.

In an embodiment, the one or more microorganisms is a bacterium and wherein the one or more microbes is a different bacterium.

In an embodiment, the EVs induce ferroptosis-like cell death of the one or more microbes of the biofilm. In an embodiment, the ferroptosis-like cell death comprises an increase in iron by the one or more microbes. In an embodiment, the ferroptosis-like cell death comprises an increase in reactive oxygen species (ROS) accumulation by the one or more microbes.

In an embodiment, the one or more microbes comprises at least one species of an ESKAPEE pathogen. In an embodiment, the ESKAPEE pathogen is Enterococcus. In an embodiment, the ESKAPEE pathogen is Enterococcus spp. In an embodiment, the Enterococcus is Enterococcus faecium. In an embodiment, the ESKAPEE pathogen is Staphylococcus aureus. In an embodiment, the S. aureus is methicillin-resistant (MRSA).

In an embodiment, the ESKAPEE pathogen is Klebsiella pneumoniae. In an embodiment, the ESKAPEE pathogen is Acinetobacter baumanii. In an embodiment, the ESKAPEE pathogen is Pseudomonas aeruginosa. In an embodiment, the P. aeruginosa is PAO1.

In an embodiment, the ESKAPEE pathogen is Enterobacter. In an embodiment, the ESKAPEE pathogen is Enterobacter spp. In an embodiment, the ESKAPEE pathogen is Escherichia coli.

In an embodiment, the one or more microbes is a bacterium. In an embodiment, the bacterium is an antibiotic-resistant bacterium. In an embodiment, the bacterium is a Gram-negative bacterium. In an embodiment, the bacterium is a Gram-positive bacterium.

In an embodiment, the bacterium is an ESKAPEE pathogen. In an embodiment, the ESKAPEE pathogen is Enterococcus. In an embodiment, the ESKAPEE pathogen is Enterococcus spp. In an embodiment, the Enterococcus is Enterococcus faecium. In an embodiment, the ESKAPEE pathogen is Staphylococcus aureus. In an embodiment, the S. aureus is methicillin-resistant (MRSA).

In an embodiment, the ESKAPEE pathogen is Klebsiella pneumoniae. In an embodiment, the ESKAPEE pathogen is Acinetobacter baumanii. In an embodiment, the ESKAPEE pathogen is Pseudomonas aeruginosa. In an embodiment, the P. aeruginosa is PAO1.

In an embodiment, the ESKAPEE pathogen is Enterobacter. In an embodiment, the ESKAPEE pathogen is Enterobacter spp. In an embodiment, the ESKAPEE pathogen is Escherichia coli.

In an embodiment, the one or more microbes is a fungus. In an embodiment, the fungus is Candida. In an embodiment, the fungus is Candida spp. In an embodiment, the fungus is Candida auris.

In an embodiment, the biofilm is present on a wound. In an embodiment, the wound is a chronic wound. In an embodiment, the wound is a surgical wound. In an embodiment, the wound is an ulcer. In an embodiment, the wound is a wound bed. In an embodiment, the wound is an organ transplant wound.

In an embodiment, the biofilm is present within a wound. In an embodiment, the wound is a chronic wound. In an embodiment, the wound is a surgical wound. In an embodiment, the wound is an ulcer. In an embodiment, the wound is a wound bed. In an embodiment, the wound is an organ transplant wound.

In an embodiment, the biofilm is present on a medical device. In an embodiment, the medical device is an implant. In an embodiment, the medical device is a contact lens. In an embodiment, the medical device is a catheter. In an embodiment, the catheter is a urinary catheter. In an embodiment, the catheter is a dialysis catheter.

In an embodiment, the medical device is a prosthetic joint. In an embodiment, the medical device is an endotracheal tube. In an embodiment, the medical device is a mechanical heart valve.

In an embodiment, the biofilm is present within a medical device. In an embodiment, the medical device is an implant. In an embodiment, the medical device is a contact lens. In an embodiment, the medical device is a catheter. In an embodiment, the catheter is a urinary catheter. In an embodiment, the catheter is a dialysis catheter.

In an embodiment, the medical device is a prosthetic joint. In an embodiment, the medical device is an endotracheal tube. In an embodiment, the medical device is a mechanical heart valve.

In an embodiment, the method further comprises administering a second treatment to inhibit the biofilm. In an embodiment, the second treatment is an antibiotic. In an embodiment, the second treatment is a fungicide. In an embodiment, the second treatment is debridement.

The following numbered embodiments are contemplated and are non-limiting:

1. A method of collecting one or more extracellular vesicles (EVs) from a composition comprising one or more microorganisms, the method comprising:
(a) culturing the one or more microorganisms, wherein the culturing comprises secretion of one or more EVs from the microorganisms;
(b) separating the EVs from the composition; and
(c) collecting the EVs separated in step (b).

2. The method of clause 1, any other suitable clause, or any combination of suitable clauses, wherein the composition comprises a microbial culture.

3. The method of clause 1, any other suitable clause, or any combination of suitable clauses, wherein the composition comprises a biofilm.

4. The method of clause 1, any other suitable clause, or any combination of suitable clauses, wherein the composition comprises a planktonic culture.

5. The method of clause 1, any other suitable clause, or any combination of suitable clauses, wherein the culturing comprises induction of a stress condition to the one or more microorganisms.

6. The method of clause 5, any other suitable clause, or any combination of suitable clauses, wherein the stress condition comprises a temperature stress condition.

7. The method of clause 6, any other suitable clause, or any combination of suitable clauses, wherein the temperature stress condition is a heat stress.

8. The method of clause 6, any other suitable clause, or any combination of suitable clauses, wherein the temperature stress condition is a decreased temperature.

9. The method of clause 5, any other suitable clause, or any combination of suitable clauses, wherein the stress condition comprises a nutrient starvation.

10. The method of clause 9, any other suitable clause, or any combination of suitable clauses, wherein the nutrient starvation comprises a reduction of carbon in the composition.

11. The method of clause 5, any other suitable clause, or any combination of suitable clauses, wherein the stress condition comprises an oxidative stress.

12. The method of clause 11, any other suitable clause, or any combination of suitable clauses, wherein the oxidative stress comprises a reactive oxygen species.

13. The method of clause 11, any other suitable clause, or any combination of suitable clauses, wherein the oxidative stress comprises a reactive nitrogen species.

14. The method of clause 5, any other suitable clause, or any combination of suitable clauses, wherein the stress condition comprises decreasing pH of the composition to below a pH of 7.

15. The method of clause 5, any other suitable clause, or any combination of suitable clauses, wherein the stress condition comprises increasing pH of the composition to above a pH of 7.

16. The method of clause 5, any other suitable clause, or any combination of suitable clauses, wherein the stress condition comprises increasing osmotic pressure of the composition to a pressure of 1,700 mosmol or higher.

17. The method of clause 5, any other suitable clause, or any combination of suitable clauses, wherein the stress condition comprises increasing the salinity of the composition.

18. The method of clause 5, any other suitable clause, or any combination of suitable clauses, wherein the stress condition comprises applying UV stress to the composition.

19. The method of clause 5, any other suitable clause, or any combination of suitable clauses, wherein the stress condition comprises introducing a chemical to the composition.

20. The method of clause 19, any other suitable clause, or any combination of suitable clauses, wherein the chemical is a fungicide.

21. The method of clause 19, any other suitable clause, or any combination of suitable clauses, wherein the chemical is an antibiotic.

22. The method of clause 19, any other suitable clause, or any combination of suitable clauses, wherein the chemical is an antimicrobial peptide.

23. The method of clause 19, any other suitable clause, or any combination of suitable clauses, wherein the chemical is a metal.

24. The method of clause 23, any other suitable clause, or any combination of suitable clauses, wherein the metal is aluminum.

25. The method of clause 23, any other suitable clause, or any combination of suitable clauses, wherein the metal is copper.

26. The method of clause 23, any other suitable clause, or any combination of suitable clauses, wherein the metal is silver.

27. The method of clause 23, any other suitable clause, or any combination of suitable clauses, wherein the metal is an iron.

28. The method of clause 27, any other suitable clause, or any combination of suitable clauses, wherein the iron is Fe²⁺.

29. The method of clause 27, any other suitable clause, or any combination of suitable clauses, wherein the iron is Fe³⁺.

30. The method of clause 1, any other suitable clause, or any combination of suitable clauses, wherein the stress condition comprises deprivation of oxygen to the composition.

31. The method of clause 1, any other suitable clause, or any combination of suitable clauses, wherein the stress condition is selected from the group consisting of a temperature stress condition, a nutrient starvation, an oxidative stress, decreasing the pH of the composition to below a pH of 7, increasing the pH of the composition to above a pH of 7, increasing the osmotic pressure of the composition, increasing the salinity of the composition, applying UV stress to the composition, introducing a chemical to the composition, deprivation of oxygen to the composition, or combinations thereof.

32. The method of clause 1, any other suitable clause, or any combination of suitable clauses, wherein the method further comprises extracting the EVs from the composition.

33. The method of clause 1, any other suitable clause, or any combination of suitable clauses, wherein the separating comprises centrifugation.

34. The method of clause 1, any other suitable clause, or any combination of suitable clauses, wherein the separating comprises a chemical gradient.

35. The method of clause 1, any other suitable clause, or any combination of suitable clauses, wherein the method further comprises concentrating the EVs.

36. The method of clause 1, any other suitable clause, or any combination of suitable clauses, wherein the one or more EVs comprise a death EV (D-EV).

37. The method of clause 1, any other suitable clause, or any combination of suitable clauses, wherein the one or more EVs consists essentially of a death EV (D-EV).

38. The method of clause 1, any other suitable clause, or any combination of suitable clauses, wherein the one or more EVs consists of a death EV (D-EV).

39. The method of clause 1, any other suitable clause, or any combination of suitable clauses, wherein the one or more microorganisms comprise a consortium of microorganisms.

40. The method of clause 39, any other suitable clause, or any combination of suitable clauses, wherein the consortium of microorganisms comprises at least one species of an ESKAPEE pathogen.

41. The method of clause 40, any other suitable clause, or any combination of suitable clauses, wherein the ESKAPEE pathogen is Enterococcus.

42. The method of clause 40, any other suitable clause, or any combination of suitable clauses, wherein the ESKAPEE pathogen is Enterococcus spp.

43. The method of clause 40, any other suitable clause, or any combination of suitable clauses, wherein the Enterococcus is Enterococcus faecium.

44. The method of clause 40, any other suitable clause, or any combination of suitable clauses, wherein the ESKAPEE pathogen is Staphylococcus aureus.

45. The method of clause 44, any other suitable clause, or any combination of suitable clauses, wherein the S. aureus is methicillin-resistant (MRSA).

46. The method of clause 40, any other suitable clause, or any combination of suitable clauses, wherein the ESKAPEE pathogen is Klebsiella pneumoniae.

47. The method of clause 40, any other suitable clause, or any combination of suitable clauses, wherein the ESKAPEE pathogen is Acinetobacter baumanii.

48. The method of clause 40, any other suitable clause, or any combination of suitable clauses, wherein the ESKAPEE pathogen is Pseudomonas aeruginosa.

49. The method of clause 48, any other suitable clause, or any combination of suitable clauses, wherein the P. aeruginosa is PAO1.

50. The method of clause 40, any other suitable clause, or any combination of suitable clauses, wherein the ESKAPEE pathogen is Enterobacter.

51. The method of clause 40, any other suitable clause, or any combination of suitable clauses, wherein the ESKAPEE pathogen is Enterobacter spp.

52. The method of clause 40, any other suitable clause, or any combination of suitable clauses, wherein the ESKAPEE pathogen is Escherichia coli.

53. The method of clause 1, any other suitable clause, or any combination of suitable clauses, wherein the one or more microorganisms is a bacterium.

54. The method of clause 53, any other suitable clause, or any combination of suitable clauses, wherein the bacterium is an antibiotic-resistant bacterium.

55. The method of clause 53, any other suitable clause, or any combination of suitable clauses, wherein the bacterium is a Gram-negative bacterium.

56. The method of clause 53, any other suitable clause, or any combination of suitable clauses, wherein the bacterium is a Gram-positive bacterium.

57. The method of clause 53, any other suitable clause, or any combination of suitable clauses, wherein the bacterium is an ESKAPEE pathogen.

58. The method of clause 57, any other suitable clause, or any combination of suitable clauses, wherein the ESKAPEE pathogen is Enterococcus.

59. The method of clause 57, any other suitable clause, or any combination of suitable clauses, wherein the ESKAPEE pathogen is Enterococcus spp.

60. The method of clause 57, any other suitable clause, or any combination of suitable clauses, wherein the Enterococcus is Enterococcus faecium.

61. The method of clause 57, any other suitable clause, or any combination of suitable clauses, wherein the ESKAPEE pathogen is Staphylococcus aureus.

62. The method of clause 61, any other suitable clause, or any combination of suitable clauses, wherein the S. aureus is methicillin-resistant (MRSA).

63. The method of clause 57, any other suitable clause, or any combination of suitable clauses, wherein the ESKAPEE pathogen is Klebsiella pneumoniae.

64. The method of clause 57, any other suitable clause, or any combination of suitable clauses, wherein the ESKAPEE pathogen is Acinetobacter baumanii.

65. The method of clause 57, any other suitable clause, or any combination of suitable clauses, wherein the ESKAPEE pathogen is Pseudomonas aeruginosa.

66. The method of clause 65, any other suitable clause, or any combination of suitable clauses, wherein the P. aeruginosa is PAO1.

67. The method of clause 57, any other suitable clause, or any combination of suitable clauses, wherein the ESKAPEE pathogen is Enterobacter.

68. The method of clause 57, any other suitable clause, or any combination of suitable clauses, wherein the ESKAPEE pathogen is Enterobacter spp.

69. The method of clause 57, any other suitable clause, or any combination of suitable clauses, wherein the ESKAPEE pathogen is Escherichia coli.

70. The method of clause 1, any other suitable clause, or any combination of suitable clauses, wherein the one or more microorganisms is a fungus.

71. The method of clause 70, any other suitable clause, or any combination of suitable clauses, wherein the fungus is Candida.

72. The method of clause 70, any other suitable clause, or any combination of suitable clauses, wherein the fungus is Candida spp.

73. The method of clause 70, any other suitable clause, or any combination of suitable clauses, wherein the fungus is Candida auris.

74. The method of clause 1, any other suitable clause, or any combination of suitable clauses, wherein the one or more microorganisms is an engineered microorganism.

75. The method of clause 74, any other suitable clause, or any combination of suitable clauses, wherein the engineered microorganism is an engineered bacterium.

76. The method of clause 74, any other suitable clause, or any combination of suitable clauses, wherein the engineered microorganism is an engineered fungus.

77. The method of clause 1, any other suitable clause, or any combination of suitable clauses, wherein the separating is selected from a group consisting of centrifugation, a chemical gradient, filtration, or a combination thereof.

78. The method of clause 1, any other suitable clause, or any combination of suitable clauses, wherein the collected EVs are subsequently lyophilized.

79. The method of clause 1, any other suitable clause, or any combination of suitable clauses, wherein the collected EVs are subsequently encapsulated.

80. An extracellular vesicle (EV) obtained by the method of claim 1.

81. The EV of clause 80, any other suitable clause, or any combination of suitable clauses, wherein the EV is lyophilized.

82. The EV of clause 80, wherein the EV is encapsulated.

83. A method of inhibiting a biofilm, the method comprising a step of applying an effective amount of one or more extracellular vesicles (EVs) collected from one or more microorganisms, where the EVs provide inhibition of the biofilm.

84. The method of clause 83, any other suitable clause, or any combination of suitable clauses, wherein the EVs are obtained by the method of any one of clauses 1 to 79.

85. The method of clause 83, any other suitable clause, or any combination of suitable clauses, wherein the inhibition comprises a reduction of growth of the biofilm.

86. The method of clause 83, any other suitable clause, or any combination of suitable clauses, wherein the inhibition comprises a reduction in size of the biofilm.

87. The method of clause 83, any other suitable clause, or any combination of suitable clauses, wherein the one or more microorganisms comprise a consortium of microorganisms.

88. The method of clause 87, any other suitable clause, or any combination of suitable clauses, wherein the consortium of microorganisms comprises at least one species of an ESKAPEE pathogen.

89. The method of clause 88, any other suitable clause, or any combination of suitable clauses, wherein the ESKAPEE pathogen is Enterococcus.

90. The method of clause 88, any other suitable clause, or any combination of suitable clauses, wherein the ESKAPEE pathogen is Enterococcus spp.

91. The method of clause 88, any other suitable clause, or any combination of suitable clauses, wherein the Enterococcus is Enterococcus faecium.

92. The method of clause 88, any other suitable clause, or any combination of suitable clauses, wherein the ESKAPEE pathogen is Staphylococcus aureus.

93. The method of clause 92, any other suitable clause, or any combination of suitable clauses, wherein the S. aureus is methicillin-resistant (MRSA).

94. The method of clause 88, any other suitable clause, or any combination of suitable clauses, wherein the ESKAPEE pathogen is Klebsiella pneumoniae.

95. The method of clause 88, any other suitable clause, or any combination of suitable clauses, wherein the ESKAPEE pathogen is Acinetobacter baumanii.

96. The method of clause 88, any other suitable clause, or any combination of suitable clauses, wherein the ESKAPEE pathogen is Pseudomonas aeruginosa.

97. The method of clause 96, any other suitable clause, or any combination of suitable clauses, wherein the P. aeruginosa is PAO1.

98. The method of clause 88, any other suitable clause, or any combination of suitable clauses, wherein the ESKAPEE pathogen is Enterobacter.

99. The method of clause 88, any other suitable clause, or any combination of suitable clauses, wherein the ESKAPEE pathogen is Enterobacter spp.

100. The method of clause 88, any other suitable clause, or any combination of suitable clauses, wherein the ESKAPEE pathogen is Escherichia coli.

101. The method of clause 83, any other suitable clause, or any combination of suitable clauses, wherein the one or more microorganisms is a bacterium.

102. The method of clause 101, any other suitable clause, or any combination of suitable clauses, wherein the bacterium is an antibiotic-resistant bacterium.

103. The method of clause 101, any other suitable clause, or any combination of suitable clauses, wherein the bacterium is a Gram-negative bacterium.

104. The method of clause 101, any other suitable clause, or any combination of suitable clauses, wherein the bacterium is a Gram-positive bacterium.

105. The method of clause 101, any other suitable clause, or any combination of suitable clauses, wherein the bacterium is an ESKAPEE pathogen.

106. The method of clause 105, any other suitable clause, or any combination of suitable clauses, wherein the ESKAPEE pathogen is Enterococcus.

107. The method of clause 105, any other suitable clause, or any combination of suitable clauses, wherein the ESKAPEE pathogen is Enterococcus spp.

108. The method of clause 105, any other suitable clause, or any combination of suitable clauses, wherein the Enterococcus is Enterococcus faecium.

109. The method of clause 105, any other suitable clause, or any combination of suitable clauses, wherein the ESKAPEE pathogen is Staphylococcus aureus.

110. The method of clause 109, any other suitable clause, or any combination of suitable clauses, wherein the S. aureus is methicillin-resistant (MRSA).

111. The method of clause 105, any other suitable clause, or any combination of suitable clauses, wherein the ESKAPEE pathogen is Klebsiella pneumoniae.

112. The method of clause 105, any other suitable clause, or any combination of suitable clauses, wherein the ESKAPEE pathogen is Acinetobacter baumanii.

113. The method of clause 105, any other suitable clause, or any combination of suitable clauses, wherein the ESKAPEE pathogen is Pseudomonas aeruginosa.

114. The method of clause 113, any other suitable clause, or any combination of suitable clauses, wherein the P. aeruginosa is PAO1.

115. The method of clause 105, any other suitable clause, or any combination of suitable clauses, wherein the ESKAPEE pathogen is Enterobacter.

116. The method of clause 105, any other suitable clause, or any combination of suitable clauses, wherein the ESKAPEE pathogen is Enterobacter spp.

117. The method of clause 105, any other suitable clause, or any combination of suitable clauses, wherein the ESKAPEE pathogen is Escherichia coli.

118. The method of clause 83, any other suitable clause, or any combination of suitable clauses, wherein the one or more microorganisms is a fungus.

119. The method of clause 118, any other suitable clause, or any combination of suitable clauses, wherein the fungus is Candida.

120. The method of clause 118, any other suitable clause, or any combination of suitable clauses, wherein the fungus is Candida spp.

121. The method of clause 118, any other suitable clause, or any combination of suitable clauses, wherein the fungus is Candida auris.

122. The method of clause 83, any other suitable clause, or any combination of suitable clauses, wherein the one or more microorganisms is an engineered microorganism.

123. The method of clause 122, any other suitable clause, or any combination of suitable clauses, wherein the engineered microorganism is an engineered bacterium.

124. The method of clause 122, any other suitable clause, or any combination of suitable clauses, wherein the engineered microorganism is an engineered fungus.

125. The method of clause 83, any other suitable clause, or any combination of suitable clauses, wherein the biofilm comprises one or more microbes.

126. The method of clause 125, any other suitable clause, or any combination of suitable clauses, wherein the EVs provide inhibition of the one or more microbes of the biofilm.

127. The method of clause 125, any other suitable clause, or any combination of suitable clauses, wherein the EVs provide inhibition of the one or more microbes of the biofilm.

128. The method of clause 125, any other suitable clause, or any combination of suitable clauses, wherein the one or more microorganisms is a bacterium and wherein the one or more microbes is a different bacterium.

129. The method of clause 125, any other suitable clause, or any combination of suitable clauses, wherein the EVs induce ferroptosis-like cell death of the one or more microbes of the biofilm.

130. The method of clause 129, any other suitable clause, or any combination of suitable clauses, wherein the ferroptosis-like cell death comprises an increase in iron by the one or more microbes.

131. The method of clause 129, any other suitable clause, or any combination of suitable clauses, wherein the ferroptosis-like cell death comprises an increase in reactive oxygen species (ROS) accumulation by the one or more microbes.

132. The method of clause 125, any other suitable clause, or any combination of suitable clauses, wherein the one or more microbes comprises at least one species of an ESKAPEE pathogen.

133. The method of clause 132, any other suitable clause, or any combination of suitable clauses, wherein the ESKAPEE pathogen is Enterococcus.

134. The method of clause 132, any other suitable clause, or any combination of suitable clauses, wherein the ESKAPEE pathogen is Enterococcus spp.

135. The method of clause 132, any other suitable clause, or any combination of suitable clauses, wherein the Enterococcus is Enterococcus faecium.

136. The method of clause 132, any other suitable clause, or any combination of suitable clauses, wherein the ESKAPEE pathogen is Staphylococcus aureus.

137. The method of clause 136, any other suitable clause, or any combination of suitable clauses, wherein the S. aureus is methicillin-resistant (MRSA).

138. The method of clause 132, any other suitable clause, or any combination of suitable clauses, wherein the ESKAPEE pathogen is Klebsiella pneumoniae.

139. The method of clause 132, any other suitable clause, or any combination of suitable clauses, wherein the ESKAPEE pathogen is Acinetobacter baumanii.

140. The method of clause 132, any other suitable clause, or any combination of suitable clauses, wherein the ESKAPEE pathogen is Pseudomonas aeruginosa.

141. The method of clause 140, any other suitable clause, or any combination of suitable clauses, wherein the P. aeruginosa is PAO1.

142. The method of clause 132, any other suitable clause, or any combination of suitable clauses, wherein the ESKAPEE pathogen is Enterobacter.

143. The method of clause 132, any other suitable clause, or any combination of suitable clauses, wherein the ESKAPEE pathogen is Enterobacter spp.

144. The method of clause 132, any other suitable clause, or any combination of suitable clauses, wherein the ESKAPEE pathogen is Escherichia coli.

145. The method of clause 125, any other suitable clause, or any combination of suitable clauses, wherein the one or more microbes is a bacterium.

146. The method of clause 145, any other suitable clause, or any combination of suitable clauses, wherein the bacterium is an antibiotic-resistant bacterium.

147. The method of clause 145, any other suitable clause, or any combination of suitable clauses, wherein the bacterium is a Gram-negative bacterium.

148. The method of clause 145, any other suitable clause, or any combination of suitable clauses, wherein the bacterium is a Gram-positive bacterium.

149. The method of clause 145, any other suitable clause, or any combination of suitable clauses, wherein the bacterium is an ESKAPEE pathogen.

150. The method of clause 149, any other suitable clause, or any combination of suitable clauses, wherein the ESKAPEE pathogen is Enterococcus.

151. The method of clause 149, any other suitable clause, or any combination of suitable clauses, wherein the ESKAPEE pathogen is Enterococcus spp.

152. The method of clause 149, any other suitable clause, or any combination of suitable clauses, wherein the Enterococcus is Enterococcus faecium.

153. The method of clause 149, any other suitable clause, or any combination of suitable clauses, wherein the ESKAPEE pathogen is Staphylococcus aureus.

154. The method of clause 153, any other suitable clause, or any combination of suitable clauses, wherein the S. aureus is methicillin-resistant (MRSA).

155. The method of clause 149, any other suitable clause, or any combination of suitable clauses, wherein the ESKAPEE pathogen is Klebsiella pneumoniae.

156. The method of clause 149, any other suitable clause, or any combination of suitable clauses, wherein the ESKAPEE pathogen is Acinetobacter baumanii.

157. The method of clause 149, any other suitable clause, or any combination of suitable clauses, wherein the ESKAPEE pathogen is Pseudomonas aeruginosa.

158. The method of clause 157, any other suitable clause, or any combination of suitable clauses, wherein the P. aeruginosa is PAO1.

159. The method of clause 149, any other suitable clause, or any combination of suitable clauses, wherein the ESKAPEE pathogen is Enterobacter.

160. The method of clause 149, any other suitable clause, or any combination of suitable clauses, wherein the ESKAPEE pathogen is Enterobacter spp.

161. The method of clause 149, any other suitable clause, or any combination of suitable clauses, wherein the ESKAPEE pathogen is Escherichia coli.

162. The method of clause 125, any other suitable clause, or any combination of suitable clauses, wherein the one or more microbes is a fungus.

163. The method of clause 162, any other suitable clause, or any combination of suitable clauses, wherein the fungus is Candida.

164. The method of clause 162, any other suitable clause, or any combination of suitable clauses, wherein the fungus is Candida spp.

165. The method of clause 162, any other suitable clause, or any combination of suitable clauses, wherein the fungus is Candida auris.

166. The method of clause 83, any other suitable clause, or any combination of suitable clauses, wherein the biofilm is present on a wound.

167. The method of clause 166, any other suitable clause, or any combination of suitable clauses, wherein the wound is a chronic wound.

168. The method of clause 166, any other suitable clause, or any combination of suitable clauses, wherein the wound is a surgical wound.

169. The method of clause 166, any other suitable clause, or any combination of suitable clauses, wherein the wound is an ulcer.

170. The method of clause 166, any other suitable clause, or any combination of suitable clauses, wherein the wound is a wound bed.

171. The method of clause 166, any other suitable clause, or any combination of suitable clauses, wherein the wound is an organ transplant wound.

172. The method of clause 83, any other suitable clause, or any combination of suitable clauses, wherein the biofilm is present within a wound.

173. The method of clause 172, any other suitable clause, or any combination of suitable clauses, wherein the wound is a chronic wound.

174. The method of clause 172, any other suitable clause, or any combination of suitable clauses, wherein the wound is a surgical wound.

175. The method of clause 172, any other suitable clause, or any combination of suitable clauses, wherein the wound is an ulcer.

176. The method of clause 172, any other suitable clause, or any combination of suitable clauses, wherein the wound is a wound bed.

177. The method of clause 172, any other suitable clause, or any combination of suitable clauses, wherein the wound is an organ transplant wound.

178. The method of clause 83, any other suitable clause, or any combination of suitable clauses, wherein the biofilm is present on a medical device.

179. The method of clause 178, any other suitable clause, or any combination of suitable clauses, wherein the medical device is an implant.

180. The method of clause 178, any other suitable clause, or any combination of suitable clauses, wherein the medical device is a contact lens.

181. The method of clause 178, any other suitable clause, or any combination of suitable clauses, wherein the medical device is a catheter.

182. The method of clause 181, any other suitable clause, or any combination of suitable clauses, wherein the catheter is a urinary catheter.

183. The method of clause 181, any other suitable clause, or any combination of suitable clauses, wherein the catheter is a dialysis catheter.

184. The method of clause 178, any other suitable clause, or any combination of suitable clauses, wherein the medical device is a prosthetic joint.

185. The method of clause 178, any other suitable clause, or any combination of suitable clauses, wherein the medical device is an endotracheal tube.

186. The method of clause 178, any other suitable clause, or any combination of suitable clauses, wherein the medical device is a mechanical heart valve.

187. The method of clause 83, any other suitable clause, or any combination of suitable clauses, wherein the biofilm is present within a medical device.

188. The method of clause 187, any other suitable clause, or any combination of suitable clauses, wherein the medical device is an implant.

189. The method of clause 187, any other suitable clause, or any combination of suitable clauses, wherein the medical device is a contact lens.

190. The method of clause 187, any other suitable clause, or any combination of suitable clauses, wherein the medical device is a catheter.

191. The method of clause 190, any other suitable clause, or any combination of suitable clauses, wherein the catheter is a urinary catheter.

192. The method of clause 190, any other suitable clause, or any combination of suitable clauses, wherein the catheter is a dialysis catheter.

193. The method of clause 187, any other suitable clause, or any combination of suitable clauses, wherein the medical device is a prosthetic joint.

194. The method of clause 187, any other suitable clause, or any combination of suitable clauses, wherein the medical device is an endotracheal tube.

195. The method of clause 187, any other suitable clause, or any combination of suitable clauses, wherein the medical device is a mechanical heart valve.

196. The method of clause 83, any other suitable clause, or any combination of suitable clauses, wherein the method further comprises administering a second treatment to inhibit the biofilm.

197. The method of clause 196, any other suitable clause, or any combination of suitable clauses, wherein the second treatment is an antibiotic.

198. The method of clause 196, any other suitable clause, or any combination of suitable clauses, wherein the second treatment is a fungicide.

199. The method of clause 196, any other suitable clause, or any combination of suitable clauses, wherein the second treatment is debridement.

200. A composition comprising one or more death extracellular vesicles (D-EVs).

201. The composition of claim 200, any other suitable clause, or any combination of suitable clauses, wherein the one or more D-EVs comprise one or more proteins selected from the group consisting of a redox cycle-regulating protein, an iron-acquisition protein, a quorum sensing (QS) protein, and any combination thereof.

202. The composition of claim 200, any other suitable clause, or any combination of suitable clauses, wherein the one or more D-EVs comprise a redox cycle-regulating protein.

203. The composition of claim 200, any other suitable clause, or any combination of suitable clauses, wherein the one or more D-EVs comprise an iron-acquisition protein.

204. The composition of claim 200, any other suitable clause, or any combination of suitable clauses, wherein the one or more D-EVs comprise a quorum sensing (QS) protein.

205. The composition of claim 200, any other suitable clause, or any combination of suitable clauses, wherein the one or more D-EVs comprise one or more proteins selected from Table 5.

206. The composition of claim 200, any other suitable clause, or any combination of suitable clauses, wherein the one or more D-EVs comprise one or more proteins selected from Table 6.

207. The composition of claim 200, any other suitable clause, or any combination of suitable clauses, wherein the one or more D-EVs comprise one or more proteins selected from Table 5 and Table 6.

208. The composition of claim 200, any other suitable clause, or any combination of suitable clauses, wherein the one or more D-EVs is isolated from a microorganism.

209. The composition of claim 208, any other suitable clause, or any combination of suitable clauses, wherein the microorganism is an ESKAPEE pathogen.

210. The composition of claim 209, any other suitable clause, or any combination of suitable clauses, wherein the ESKAPEE pathogen is Enterococcus.

211. The composition of claim 209, any other suitable clause, or any combination of suitable clauses, wherein the ESKAPEE pathogen is Enterococcus spp.

212. The composition of claim 211, any other suitable clause, or any combination of suitable clauses, wherein the Enterococcus is Enterococcus faecium.

213. The composition of claim 209, any other suitable clause, or any combination of suitable clauses, wherein the ESKAPEE pathogen is Staphylococcus aureus.

214. The composition of claim 213, any other suitable clause, or any combination of suitable clauses, wherein the S. aureus is methicillin-resistant (MRSA).

215. The composition of claim 209, any other suitable clause, or any combination of suitable clauses, wherein the ESKAPEE pathogen is Klebsiella pneumoniae.

216. The composition of claim 209, any other suitable clause, or any combination of suitable clauses, wherein the ESKAPEE pathogen is Acinetobacter baumanii.

217. The composition of claim 209, any other suitable clause, or any combination of suitable clauses, wherein the ESKAPEE pathogen is Pseudomonas aeruginosa.

218. The composition of claim 217, any other suitable clause, or any combination of suitable clauses, wherein the P. aeruginosa is PAO1.

219. The composition of claim 209, any other suitable clause, or any combination of suitable clauses, wherein the ESKAPEE pathogen is Enterobacter.

220. The composition of claim 209, any other suitable clause, or any combination of suitable clauses, wherein the ESKAPEE pathogen is Enterobacter spp.

221. The composition of claim 209, any other suitable clause, or any combination of suitable clauses, wherein the ESKAPEE pathogen is Escherichia coli.

222. The composition of claim 208, any other suitable clause, or any combination of suitable clauses, wherein the microorganism is a bacterium.

223. The composition of claim 222, any other suitable clause, or any combination of suitable clauses, wherein the bacterium is an antibiotic-resistant bacterium.

224. The composition of claim 222, any other suitable clause, or any combination of suitable clauses, wherein the bacterium is a Gram-negative bacterium.

225. The composition of claim 222, any other suitable clause, or any combination of suitable clauses, wherein the bacterium is a Gram-positive bacterium.

226. The composition of claim 222, any other suitable clause, or any combination of suitable clauses, wherein the bacterium is an ESKAPEE pathogen.

227. The composition of claim 208, any other suitable clause, or any combination of suitable clauses, wherein the microorganism is a fungus.

228. The composition of claim 227, any other suitable clause, or any combination of suitable clauses, wherein the fungus is Candida.

229. The composition of claim 227, any other suitable clause, or any combination of suitable clauses, wherein the fungus is Candida spp.

230. The composition of claim 227, any other suitable clause, or any combination of suitable clauses, wherein the fungus is Candida auris.

231. The composition of claim 200, any other suitable clause, or any combination of suitable clauses, wherein the one or more D-EVs is isolated from a microorganism under a stress-induced condition.

232. The composition of claim 200, any other suitable clause, or any combination of suitable clauses, wherein the composition is an antimicrobial composition.

233. A pharmaceutical composition comprising i) one or more death extracellular vesicles (D-EVs) and ii) a pharmaceutically acceptable carrier.

234. The pharmaceutical composition of claim 233, any other suitable clause, or any combination of suitable clauses, wherein the composition is any one of the compositions of clauses 200 to 232.

235. The pharmaceutical composition of claim 233, any other suitable clause, or any combination of suitable clauses, wherein the pharmaceutical composition further comprises a metal ion.

236. The pharmaceutical composition of claim 235, any other suitable clause, or any combination of suitable clauses, wherein the metal is an iron.

237. The pharmaceutical composition of claim 236, any other suitable clause, or any combination of suitable clauses, wherein the iron is Fe²⁺.

238. The pharmaceutical composition of claim 236, any other suitable clause, or any combination of suitable clauses, wherein the iron is Fe³⁺.

239. The pharmaceutical composition of claim 233, any other suitable clause, or any combination of suitable clauses, wherein the pharmaceutical composition further comprises an iron chelator.

240. The pharmaceutical composition of claim 233, any other suitable clause, or any combination of suitable clauses, wherein the pharmaceutical composition further comprises a second therapeutic agent.

241. The pharmaceutical composition of claim 240, any other suitable clause, or any combination of suitable clauses, wherein the second therapeutic agent is an antibiotic.

242. The pharmaceutical composition of claim 240, any other suitable clause, or any combination of suitable clauses, wherein the second therapeutic agent is a fungicide.

243. The pharmaceutical composition of claim 233, any other suitable clause, or any combination of suitable clauses, wherein the pharmaceutical composition is a topical formulation.

244. The pharmaceutical composition of claim 233, any other suitable clause, or any combination of suitable clauses, wherein the pharmaceutical composition is a parenteral formulation.

245. The pharmaceutical composition of claim 233, any other suitable clause, or any combination of suitable clauses, wherein the pharmaceutical composition is an aerosolized formulation.

246. A method of treating an infection in a patient, the method comprising a step of administering a therapeutically effective amount of a pharmaceutical composition comprising i) one or more death extracellular vesicles (D-EVs) and ii) a pharmaceutically acceptable carrier to the patient, wherein the pharmaceutical composition provides treatment of the infection.

247. The method of claim 246, any other suitable clause, or any combination of suitable clauses, wherein the pharmaceutical composition is any one of the pharmaceutical compositions of clauses 233 to 245.

248. The method of claim 246, any other suitable clause, or any combination of suitable clauses, wherein the infection is caused by a microbe.

249. The method of claim 248, any other suitable clause, or any combination of suitable clauses, wherein the microbe is an ESKAPEE pathogen.

250. The method of claim 249, any other suitable clause, or any combination of suitable clauses, wherein the ESKAPEE pathogen is Enterococcus.

251. The method of claim 249, any other suitable clause, or any combination of suitable clauses, wherein the ESKAPEE pathogen is Enterococcus spp.

252. The method of claim 251, any other suitable clause, or any combination of suitable clauses, wherein the Enterococcus is Enterococcus faecium.

253. The method of claim 249, any other suitable clause, or any combination of suitable clauses, wherein the ESKAPEE pathogen is Staphylococcus aureus.

254. The method of claim 253, any other suitable clause, or any combination of suitable clauses, wherein the S. aureus is methicillin-resistant (MRSA).

255. The method of claim 249, any other suitable clause, or any combination of suitable clauses, wherein the ESKAPEE pathogen is Klebsiella pneumoniae.

256. The method of claim 249, any other suitable clause, or any combination of suitable clauses, wherein the ESKAPEE pathogen is Acinetobacter baumanii.

257. The method of claim 249, any other suitable clause, or any combination of suitable clauses, wherein the ESKAPEE pathogen is Pseudomonas aeruginosa.

258. The method of claim 257, any other suitable clause, or any combination of suitable clauses, wherein the P. aeruginosa is PAO1.

259. The method of claim 249, any other suitable clause, or any combination of suitable clauses, wherein the ESKAPEE pathogen is Enterobacter.

260. The method of claim 249, any other suitable clause, or any combination of suitable clauses, wherein the ESKAPEE pathogen is Enterobacter spp.

261. The method of claim 249, any other suitable clause, or any combination of suitable clauses, wherein the ESKAPEE pathogen is Escherichia coli.

262. The method of claim 248, any other suitable clause, or any combination of suitable clauses, wherein the microbe is a bacterium.

263. The method of claim 262, any other suitable clause, or any combination of suitable clauses, wherein the bacterium is an antibiotic-resistant bacterium.

264. The method of claim 262, any other suitable clause, or any combination of suitable clauses, wherein the bacterium is a Gram-negative bacterium.

265. The method of claim 262, any other suitable clause, or any combination of suitable clauses, wherein the bacterium is a Gram-positive bacterium.

266. The method of claim 262, any other suitable clause, or any combination of suitable clauses, wherein the bacterium is an ESKAPEE pathogen.

267. The method of claim 248, any other suitable clause, or any combination of suitable clauses, wherein the microbe is a fungus.

268. The method of claim 267, any other suitable clause, or any combination of suitable clauses, wherein the fungus is Candida.

269. The method of claim 267, any other suitable clause, or any combination of suitable clauses, wherein the fungus is Candida spp.

270. The method of claim 267, any other suitable clause, or any combination of suitable clauses, wherein the fungus is Candida auris.

271. The method of claim 248, any other suitable clause, or any combination of suitable clauses, wherein the treatment of the infection comprises growth inhibition of the microbe.

272. The method of claim 248, any other suitable clause, or any combination of suitable clauses, wherein the treatment of the infection comprises reduction of growth of a biofilm comprising the microbe.

273. The method of claim 248, any other suitable clause, or any combination of suitable clauses, wherein the treatment of the infection comprises reduction of size of a biofilm comprising the microbe.

274. The method of claim 248, any other suitable clause, or any combination of suitable clauses, wherein the treatment of the infection comprises reduction of adhesion of the microbe.

275. The method of claim 248, any other suitable clause, or any combination of suitable clauses, wherein the treatment of the infection comprises ROS-based ferroptosis of the microbe.

276. The method of claim 248, any other suitable clause, or any combination of suitable clauses, wherein the treatment of the infection comprises an increase of ROS accumulation in the microbe.

277. The method of claim 248, any other suitable clause, or any combination of suitable clauses, wherein the treatment of the infection comprises an increase in iron accumulation by the microbe.

278. The method of claim 248, any other suitable clause, or any combination of suitable clauses, wherein the treatment of the infection comprises disruption of iron metabolism of the microbe.

279. The method of claim 248, any other suitable clause, or any combination of suitable clauses, wherein the treatment of the infection comprises increased susceptibility of the microbe to oxidative stress.

280. The method of claim 248, any other suitable clause, or any combination of suitable clauses, wherein the treatment of the infection comprises induction of oxidative stress-induced cell death of the microbe.

281. The method of claim 248, any other suitable clause, or any combination of suitable clauses, wherein the treatment of the infection comprises disruption of quorum sensing of the microbe.

282. The method of claim 246, any other suitable clause, or any combination of suitable clauses, wherein the method further comprises administration of a metal ion to the patient.

283. The method of claim 282, any other suitable clause, or any combination of suitable clauses, wherein the metal is an iron.

284. The method of claim 283, any other suitable clause, or any combination of suitable clauses, wherein the iron is Fe²⁺.

285. The method of claim 283, any other suitable clause, or any combination of suitable clauses, wherein the iron is Fe³⁺.

286. The method of claim 246, any other suitable clause, or any combination of suitable clauses, wherein the method further comprises administration of an iron chelator to the patient.

287. The method of claim 246, any other suitable clause, or any combination of suitable clauses, wherein the method further comprises administration of a second therapeutic agent to the patient.

288. The method of claim 287, any other suitable clause, or any combination of suitable clauses, wherein the second therapeutic agent is an antibiotic.

289. The method of claim 287, any other suitable clause, or any combination of suitable clauses, wherein the second therapeutic agent is a fungicide.

290. The method of claim 287, any other suitable clause, or any combination of suitable clauses, wherein the second therapeutic agent is a debridement.

291. The method of claim 246, any other suitable clause, or any combination of suitable clauses, wherein the administration is a topical administration.

292. The method of claim 246, any other suitable clause, or any combination of suitable clauses, wherein the administration is a parenteral administration.

293. The method of claim 246, any other suitable clause, or any combination of suitable clauses, wherein the administration is an aerosolized administration.

294. The method of claim 246, any other suitable clause, or any combination of suitable clauses, wherein the infection is a wound.

295. The method of claim 294, any other suitable clause, or any combination of suitable clauses, wherein the wound is a chronic wound.

296. The method of claim 294, any other suitable clause, or any combination of suitable clauses, wherein the wound is a surgical wound.

297. The method of claim 294, any other suitable clause, or any combination of suitable clauses, wherein the wound is an ulcer.

298. The method of claim 294, any other suitable clause, or any combination of suitable clauses, wherein the wound is a wound bed.

299. The method of claim 294, any other suitable clause, or any combination of suitable clauses, wherein the wound is an organ transplant wound.

300. The method of claim 246, any other suitable clause, or any combination of suitable clauses, wherein the infection is a burn.

301. The method of claim 246, any other suitable clause, or any combination of suitable clauses, wherein the infection is a pulmonary infection.

302. The method of claim 301, any other suitable clause, or any combination of suitable clauses, wherein the pulmonary infection is cystic fibrosis.

303. The method of claim 301, any other suitable clause, or any combination of suitable clauses, wherein the pulmonary infection is pneumonia.

304. The method of claim 301, any other suitable clause, or any combination of suitable clauses, wherein the pulmonary infection is ventilator-associated pneumonia.

In the description herein, a word appearing in the singular encompasses its plural counterpart, and a word appearing in the plural encompasses its singular counterpart, unless implicitly or explicitly understood or stated otherwise. Furthermore, it is understood that for any given component or embodiment, any of the possible candidates or alternatives listed for that component may generally be used individually or in combination with one another, unless implicitly or explicitly understood or stated otherwise. Moreover, the figures are not necessarily drawn to scale, wherein some of the elements may be drawn merely for clarity. Also, reference numerals may be repeated among the various figures to show corresponding or analogous elements. Additionally, any list of such candidates or alternatives is merely illustrative, not limiting, unless implicitly or explicitly understood or stated otherwise. In addition, unless otherwise indicated, numbers expressing quantities of ingredients, constituents, reaction conditions and so forth used in the specification and claims are to be understood as being modified by the term “about.”

Accordingly, unless indicated to the contrary, the numerical parameters set forth in the specification and attached claims are approximations that may vary depending upon the desired properties sought to be obtained by the subject matter presented herein. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the subject matter presented herein are approximations, the numerical values set forth in the specific examples are reported as precisely as possible. Any numerical values, however, inherently contain certain errors necessarily resulting from the standard deviation found in their respective testing measurements.

### EXAMPLES

### Example 1

### Exemplary Materials and Methods Used in the Examples.

### Biofilms

P. aeruginosa (PAO1) from a −80° C. freezer was streaked on tryptic soy agar (TSA, BD Difco™ Dehydrated Culture Media, DF0369-17-6) and incubated overnight at 37° C. The P. aeruginosa was maintained on TSA plates at 4° C. and an inoculum was prepared by transferring a touch from this plate into 10 mL of tryptic soy broth (BD Bacto™) and incubated overnight. Then, a volume of 100 μL (OD₆₀₀=0.1) was used to seed 47-mm sterilized filter membranes (Membrane Solutions MCE Gridded Membrane Filter, Mixed Cellulose Esters Membrane Filter, Pore Size: 0.45 μm) on TSA petri dishes. The plates were incubated at either 37° C. or 33° C. At desired time points (0, 1, 2, 4, 8, 12, 16, 24, 32, 48, 72, 85, 96, and 120 h), the biofilm was suspended in 0.9% NaCl to prepare serial dilutions for colony forming unit (CFU) counting. The viable cell concentration (CFU/cm²) was calculated according to the literature. All experiments were done under aseptic conditions. TSA and TSB were autoclaved at 121° C. and 1.5 psa for 50 min/L.

### Extracellular Vesicle Extraction

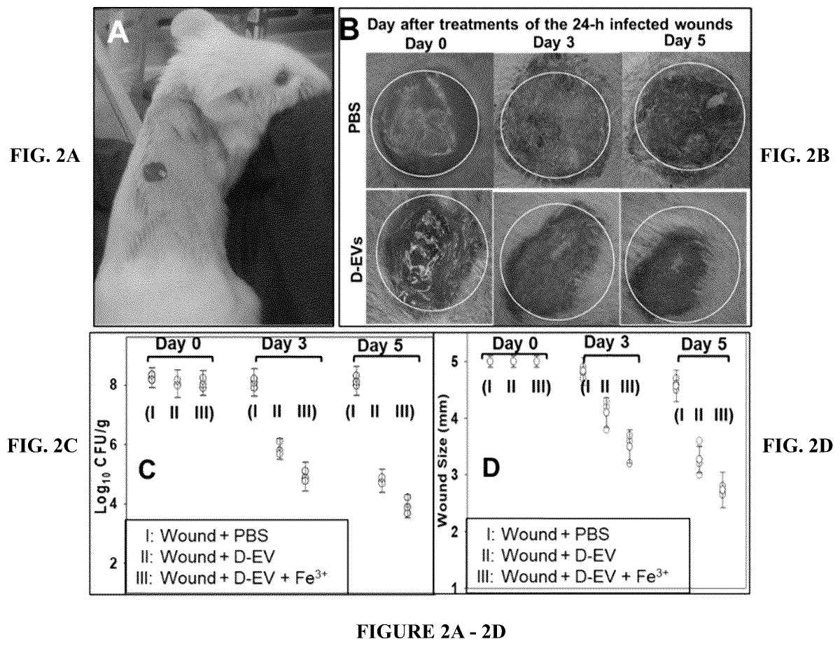

Biofilms for extracellular vesicle extraction were grown at 33° C. on 47-mm sterilized membrane paper (Membrane Solutions MCE Gridded Membrane Filter, Mixed Cellulose Esters Membrane Filter, Pore Size: 0.45 μm) on TSA petri dishes for 8 hours or 96 hours. EVs were isolated and purified from P. aeruginosa PAO1 biofilms using a conventional differential centrifugation protocol. Briefly, multiple 47-mm sterilized membrane papers were inoculated with 4.4×10⁵±7.3×10² CFU/cm² on TSA plates and incubated. When biofilms reached their exponential growth phase, their EVs were extracted and defined as growth EVs (G-EVs). Similarly, when biofilms reached their death/survival growth phase, their EVs were extracted and defined as death EVs (D-EVs). These processes are illustrated in FIG. 2. For extracellular vesicle extraction, the biofilms were removed from the membrane and then suspended in TSB. The cell debris was removed by centrifugation at 10,000 g for 30 min at 4° C. using a benchtop centrifuge (Biofuge, HERAEUS). Then, the supernatant was collected and filtered using a 0.45 μm syringe filter (GenClone 25-246, Syringe Filters, PES, 30 mm Diameter, Sterile, Cat #: 25-246). The filtrate was ultracentrifuged (Optima LE-80K Ultracentrifuge, Beckman) at 150,000 g for 2 hours at 4° C., twice, and filtered with a 0.22 μm filter (GenClone 25-244, Syringe Filters, PES, 30 mm Diameter, Sterile, Cat #: 25-244) after the first run to remove macrovesicles >200 μm in size. PBS was used as a buffer to suspend the pellets after each ultracentrifugation run. The extracted EVs were stored at −20° C. for further analysis or use.

### Testing Activity of EVs Against Biofilm at Different Ages

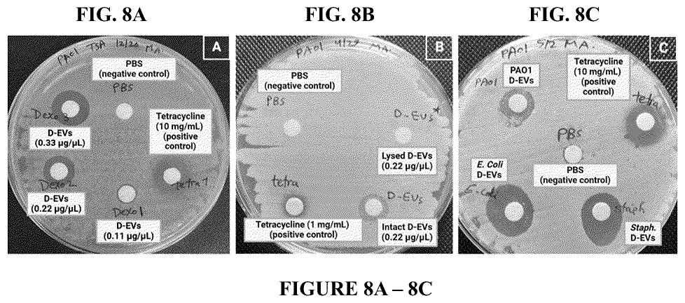

To investigate the functional effect of extracted EVs on the growth of P. aeruginosa PAO1 biofilms, the research team used the Kirby-Bauer disk diffusion susceptibility test. Briefly, an inoculum was prepared by activating a touch from the P. aeruginosa PAO1-TSA plates in TSB overnight. Then, the bacterial suspension was adjusted to OD₆₀₀˜0.1, and the adjusted suspension was spread onto TSA plates using 6-inch sterilized cotton swabs and left for 2 hours. Discs were loaded with 25 μL of PBS; tetracycline (Cat #87128, Sigma-Aldrich) at 0.033 μg/μL, 0.22 μg/μL, and 1 μg/μL; G-EVs at 0.037 μg/μL; and D-EVs at 0.11 μg/μL, 0.22 μg/μL, and 0.33 μg/μL, respectively. The treated discs were placed on the bacteria-seeded TSA plates and incubated at 37° C. overnight (FIG. 8A-8C).

To quantify the effects of G-EVs and D-EVs on the growth behavior of P. aeruginosa PAO1 biofilms, biofilms grown for 2 hr. (lag phase), 8 hr. (exponential phase), and 24 hr. (stationary phase) were tested. Briefly, an inoculum was prepared by transferring a touch from the P. aeruginosa PAO1-TSA plates into 10 ml of TSB and incubating the plates at 37° C. overnight. A volume of 2.5 μL (OD₆₀₀˜0.5) was used to seed sterile 13-mm polycarbonate membranes (WHA10417401, Sigma-Aldrich) on TSA plates and incubated at 37° C. Biofilms grown for 2 hr. were treated with one dose of G-EVs (0.028 μg/μL) or D-EVs (0.33 μg/μL) and incubated at 37° C. for 24 hours. Biofilms grown for 8 hr. were treated with one dose of G-EVs (0.037 μg/μL) or D-EVs (0.33 μg/μL) and incubated at 37° C. for 24 hours. Three doses of D-EVs (0.33 μg/μL) were applied (one dose every 24 h) to biofilms grown for 24 h, and the plates were incubated at 37° C. Then viable cells were counted and reported as CFU/cm².

### Bacterial Cells, Biofilms and EV Imaging

The research team used a Leica SP-5 confocal laser scanning microscope to image biofilms grown for 8 h. The biofilms, planktonic cells and controls were stained with LIVE/DEAD BacLight Bacterial Viability Kits following published literature. Excitations/emissions of 480/500 nm for SYTO 9 and 490/635 nm for propidium iodide were used.

Transmission electron microscopy (TEM, FEI Tecnai G2 20 Twin equipped with a 200 KV LaB6 electron source) was used to image single cells and EVs. P. aeruginosa PAO1 biofilms grown for 8 hr. were incubated with D-EVs for 24 hours. Cells were suspended in PBS for 5 minutes; then, cells were collected, followed by adding PBS and osmium at 3:1 (v:v). The mixture was then incubated at 4° C. overnight. After the resin was removed, the samples were washed with distilled water and exposed to a series of ethanol concentrations (30%, 50%, 70%, 90%, and 100%). Subsequently, the cells were mixed with Spurr in propylene oxide and left on the shaker overnight. The next day, 100% Spurr was added, and the samples were kept at 60° C. for 24 hours. Blocks were trimmed, and 70 nm thin sections were prepared and loaded onto formvar/carbon-coated copper EM grids with a thickness of 200 nm. The grids were positively stained with 2% uranyl acetate and lead. Grids were examined using TEM.

The direct interactions between G-EVs and bacterial cells were investigated using TEM and CLSM. To investigate using TEM, 8-h P. aeruginosa PAO1 biofilms were incubated with G-EVs for 24 hours. Four μL of the cell sample were loaded onto formvar/carbon-coated copper EM grids with a thickness of 200 nm. The grids were then stained with 4 μL of 2% uranyl acetate and examined using TEM. For CLSM, 2-hr. P. aeruginosa PAO1 biofilms were incubated with 10 μL of DAPI dye-labelled G-EVs at 37° C. in the dark for 24 hours. DAPI (diamidino-2-phenylindole) stains extracellular vesicle DNA. Five μL of the treated cell suspension were loaded onto a glass slide and examined with CLSM using an excitation/emission wavelength of 359/461 nm.

### Qualitative Fluorescence-Based Assay of EVs

EVs were labeled with Vybrant™ DiI Cell-Labeling Solution according to the manufacturer's protocol. To prepare the staining medium, 5 μL of the labeling solution is added to 1 mL of PBS. The EVs are then incubated with the staining medium at 37° C. for 20 minutes. After incubation, a small volume (5 μL) of the stained suspension is placed on a glass slide and examined under a confocal laser scanning microscope (Leica SP-5 Confocal Laser Scanning Microscope). The excitation and emission wavelengths used for imaging are 549 and 565 nm, respectively. This method can provide valuable insights into the cellular uptake and distribution of EVs labeled with the fluorescent dye.

### Quantitative Protein-Based Analysis of EVs

To quantify the protein concentration of the extracted extracellular vesicle samples, a modified bicinchoninic acid assay (BCA) protocol was used (Pierce™ BCA Protein Assay Kit, Thermo Fisher Scientific). The protocol involved denaturing equal amounts of extracted EVs (25 μL) using RIPA 5× for 30 min on ice, followed by protein precipitation after the addition of trichloroacetic acid (TCA, Sigma-Aldrich, 91228), (v:v, 1:0.25 of sample:TCA). The resulting precipitate was collected by centrifugation at 6000 rpm for 5 min and washed with cold acetone. The precipitate was then dissolved in PBS (25 μL) and mixed with an equal amount of the working solution (50 parts reagent A, 48 parts reagent B, and 2 parts reagent C) in a 96-well plate. The plate was incubated at 37° C. for 2 hours, and the absorbance was measured at 562 nm using a Cytation™ 5 plate reader.

### Proteomic Analysis

Proteomic identifications of extracted G-EVs and D-EVs were done using Proteome Discoverer (Thermo Orbitrap Fusion Tribrid). A 100 μL volume of EVs was centrifuged for 1 hr. at 4° C. and 150,000×g. The pellet was dissolved in 50 μL of 6 M guanidine hydrochloride (GuHCL, Thermo Scientific™, AAJ6078622) and placed on the shaker for 20 min at 1000 rpm; then, samples were sonicated 4 times for 10 s with a 2-min ice cooling interval and stored at −70° C.

### Reactive Oxygen Species Detection

The research team used a Cellular ROS Assay Kit (Red) (ab186027) to measure ROS in biofilm samples following the modified manufacturer's protocol (Abcam). Briefly, A 25 μL (OD₆₀₀=0.1) sample was transferred to each well in a 96-well plate and incubated for 7 h. A 100 μL volume of ROS working solution was added to each well and incubated for 1 h, followed by the addition of test samples (D-EVs (0.098 μg/μL), tetracycline (10 mg/ml), or PBS). The plate was placed in the plate reader for immediate monitoring of the change in ROS levels by measuring the fluorescence increase at excitation/emission 520/605 nm (cutoff 590 nm). The ROS working solution was prepared by mixing 40 μL of DMSO with the red stain; then, 5 μL of the mixture was mixed with 2.5 ml of buffer.

### Statistical Analysis

All experiments were done in biological replicates, and the number of biological replicates is given for each experiment. The data were averaged and are presented as the standard error of the mean. The statistical significance of the differences between treatments was determined using the Wilcoxon rank sum test. A p-value less than 0.05 was considered significant.

### Example 2

### Biofilm-Derived EVs to Control Biofilm Formation and Development.

Bacterial biofilms are complex surface-attached communities of bacteria held together by self-produced polymer matrices. The formation of biofilms is a complex process and involves multiple stages. As they enter a different growth stage, bacteria quickly respond to changes in the environment to propagate or survive by rapidly communicating with neighboring cells and reorganizing their intracellular physiological processes. Bacterial EVs are believed to be one of the key players in bacterial intercellular communications.

P. aeruginosa is an antibiotic-resistant Gram-negative organism and has been extensively studied for the production and characterization of EVs secreted by planktonic cells or biofilms. These studies provide a basis to understand the different pathways of EV secretions, the role of EVs in trafficking cellular signals such as quorum sensing signals to facilitate cellular activities in planktonic cells and biofilms, the biogenesis of biofilm EVs, and proteome profiles of the EVs and extracellular matrix of P. aeruginosa biofilms. Despite this extensive reported research, an understanding of the comprehensive roles of EVs in controlling P. aeruginosa biofilm growth is still elusive because the vesicle components and functional roles strongly depend on the conditions of biofilm growth stage and EV secretions.

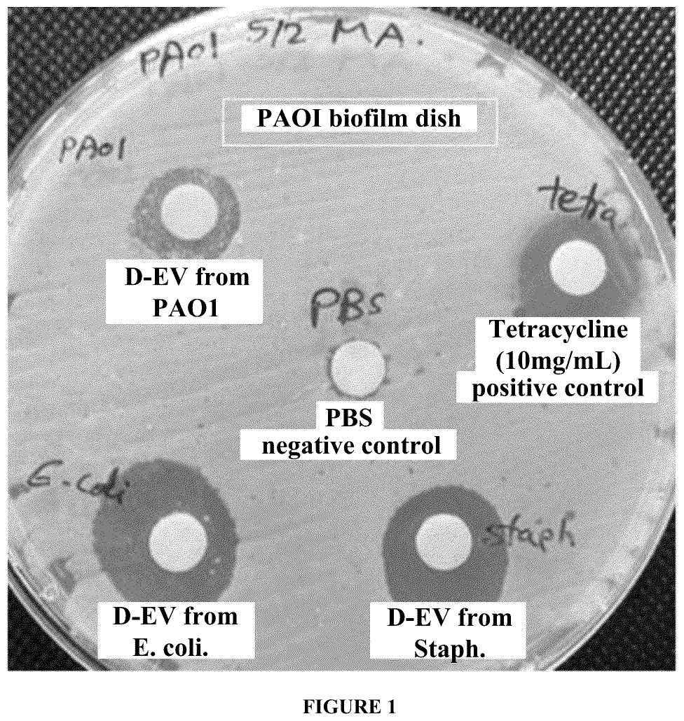

Based on understandings of EVs, it was reasoned that the role of bacterial EVs in biofilm development is culture condition dependent, i.e., that the EVs secreted by biofilms in their exponential growth phase and in their survival/death phase would promote biofilm growth and inhibit biofilm growth/formation, respectively. Because this is an unpredictable field, further characterization and study was necessary. To test this effect, the team investigated whether EVs derived from Gram-negative P. aeruginosa (PAO1) biofilms at different developmental stages can be utilized to control biofilm formation and development. These stage-dependent EVs are referred to as conditional bacterial EVs. Briefly, the team extracted EVs released by PAO1 biofilms at their exponential growth stage (G-EVs) and their survival/death phase (D-EVs), respectively. The team then examined how these conditional EVs control PAO1 biofilm growth under different conditions. The results of this study demonstrate the potential of the conditional bacterial EVs as alternative antibiotic agents for addressing the issue of antibiotic resistance currently facing society. Though this is an unpredictable art, work continues to show feasibility for other wound biofilm and non-wound biofilm applications, starting with those involving MRSA, E. coli, and ESKAPE pathogens. FIG. 1 shows a qualitative study of potential to use D-EVs from one bacterial species to inhibit biofilms formed by other bacterial species, demonstrating cross-inhibition. Table 1 summarizes cross-inhibition effects of D-EVs from E. coli, P. aeruginosa PAO1, and S. aureus on the biofilm growth of each individual bacterial species. The concentration of the D-EVs were 0.33 μg/μL, 0.35 μg/μL, and 0.37 μg/μL, respectively.

TABLE 1
Efficacy of cross inhibition of the D-
EVs from different bacteria species.

| Biofilms formed by | E. coli | P. aeruginosa (PAO1) | S. aureus |
|---|---|---|---|
| D-EVs (E. coli) | ++++ | ++++ | ++++ |
| D-EVs (P. aeruginosa) | ++++ | ++++ | ++++ |
| D-EVs (S. aureus) | +++ | ++++ | ++++ |

(++++: more effective inhibition; +: less effective inhibition)

Initial feasibility of D-EVs prevention of wound biofilm formation, and treatment of existing biofilms has also been demonstrated for murine wound infection models. Tests are shown in FIG. 2. The results showed that (1) when a single dose of D-EVs was applied to 0 hr. P. aeruginosa PAO1 infected wounds, the bacterial load was >˜4-log lower than that of the control (treated with PBS) after 3 days of healing (FIG. 2C); (2) when multiple doses of D-EVs were applied to wounds with preexisting PAO1 biofilms (24-h infected wounds), the bacterial load was reduced by >2−log₁₀ after 3 days of healing compared to the control (FIG. 2D); and (3) in the presence of 25 μM of Fe³⁺, D-EVs further reduced the bacterial load by 1−log₁₀ (FIG. 2D) and reduced the wound size (FIG. 2A). In addition, wound closure improved in the presence of D-EVs and improved even faster in the presence of D-EVs+Fe³⁺. These results strongly demonstrate the potential of D-EVs for preventing wound biofilm growth and treating preexisting biofilms.

Exploration of the roles of EVs secreted by P. aeruginosa at different developmental stages in controlling biofilm growth. Study results show that EVs secreted by P. aeruginosa biofilms during their exponential growth phase (G-EVs) enhance biofilm growth. In contrast, EVs secreted by P. aeruginosa biofilms during their death/survival phase (D-EVs) can effectively inhibit/eliminate P. aeruginosa PAO1 biofilms up to 4.8−log₁₀ CFU/cm². The inhibition effectiveness of D-EVs against P. aeruginosa biofilms grown for 96 hours improved further in the presence of 10-50 μM Fe³⁺ ions. Proteomic analysis suggests the inhibition involves an iron-dependent ferroptosis mechanism. This study is the first to report the functional role of bacterial EVs in bacterial growth, which depends on the developmental stage of the parent bacteria. The finding of D-EV-activated ferroptosis-based bacterial death has significant implications for applications involving preventing antibiotic resistance in biofilms and treating antibiotic resistant biofilms.

Highlights: (1) regulatory roles of bacterial EVs in biofilm growth are culture condition dependent; (2) D-EVs can prevent biofilm formation and treat preexisting biofilms; (3) ferric ions enhance the power of the D-EVs in treating resilient aged biofilms; and (4) evidence suggests ferroptosis involvement in the action of the D-EVs against biofilms.

Abbreviation: PAO1: P. aeruginosa PAO1; G-EVs: EVs secreted by P. aeruginosa biofilms during their exponential growth phase; D-EV: EVs secreted by P. aeruginosa biofilms during their death/survival phase; CLSM: confocal laser scanning microscopy; TEM: transmission electron microscopy.

### Example 3

### Biofilm-Derived EVs to Control Biofilm Formation and Development.

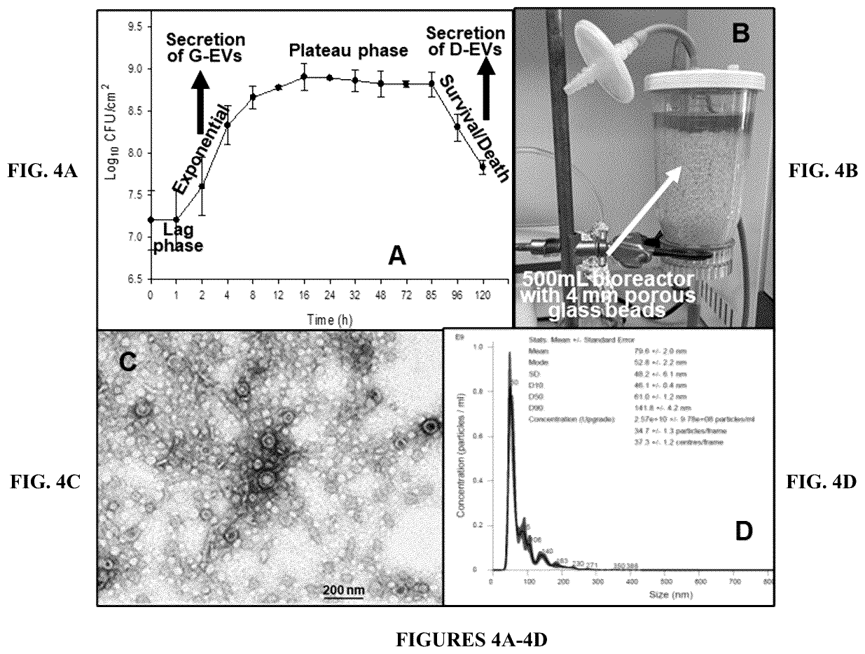

FIG. 4A shows P. aeruginosa PAO1 biofilm growth curves at 33° C. and 37° C. The growth curve for 33° C. looks similar to a typical growth curve observed for shaken or well-mixed planktonic cells. When growing at 37° C., P. aeruginosa PAO1 biofilm reached a steady cell number of ˜7.7 CFU/cm² at the 16th hour and the plateau phase lasted until the 120th hour. However, the survival/death phase was not distinguishable before the 120th hour for biofilms grown at 37° C. Under a 33° C. culture condition, PAO1 biofilm growth clearly showed four growth stages: lag, exponential growth, plateau, and survival/death, with a steady cell number of ˜6 CFU/cm² in the plateau phase. Based on these observations, in this study, all conditional EVs were extracted from P. aeruginosa PAO1 biofilms cultured under the 33° C. condition. However, all functional tests of the obtained EVs on P. aeruginosa PAO1 biofilm growth were performed under the 37° C. culture condition. It is noted that the colony biofilm model used should have heterogeneity. For example, the cells in the center may be less metabolically active or dying, while at the edge, metabolic activity is expected to be higher. Quantification of the average viable cell numbers results in the growth curve given in FIG. 4A, which represents the physiological state of the majority of the cells. The PAO1 biofilms were cultured in a 500 mL fixed bed biofilm reactor filled with 4 mm borosilicate porous glass 3.3 ROBU beads (FIG. 4B). These porous beads have high biocompatibility and a high electrical surface charge (Zeta-Potential), which promotes bacterial biofilm attachment.

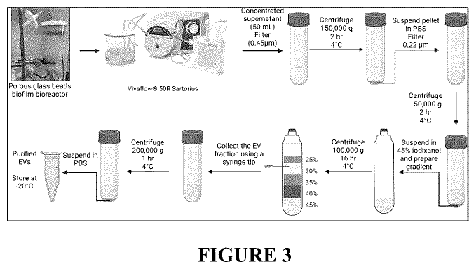

G-EVs or D-EVs of P. aeruginosa PAO1 biofilms were extracted from biofilms grown at 33° C. The biofilms were collected at specific developmental stages, as indicated in FIG. 4A, using conventional differential centrifugation protocols, which are given in the Materials and Methods section and summarized in FIG. 3. Using this protocol, a total of 874.1±20 μg EV proteins could be extracted from membrane biofilms with an initial cell concentration of 4.4×10⁵±7.3×10² CFU/cm². The sizes and particle numbers of the purified D-EVs samples were characterized using transmission electron microscopy (TEM) (FIG. 4C) and a Zetasizer NS300 (Malvern Panalytical) (FIG. 4D).

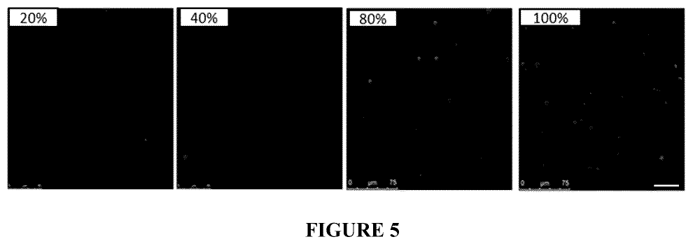

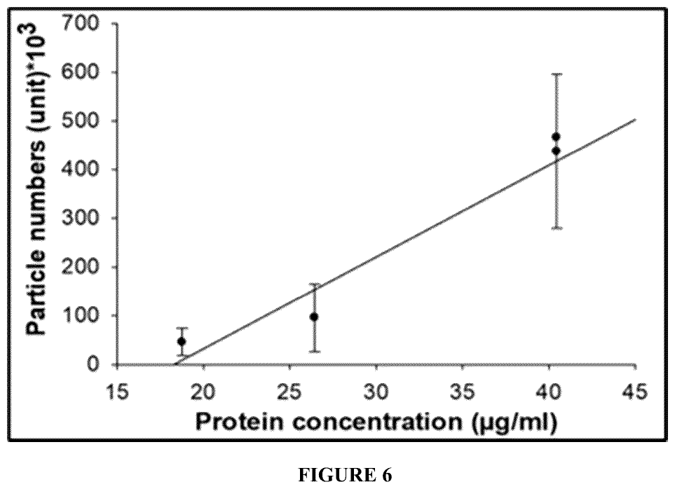

The protein concentration of the D-EVs and the number of D-EV particles in a solution sample were correlated using stained extracellular vesicle samples and known protein concentrations (see Example 7, below). The results (shown in FIG. 5 and FIG. 6 established the correlation between the protein concentrations of the D-EVs and their average particle numbers in a solution, with every D-EV particle equivalent to an average extracellular vesicle protein content of 0.00188±8×10⁴ μg/ml.

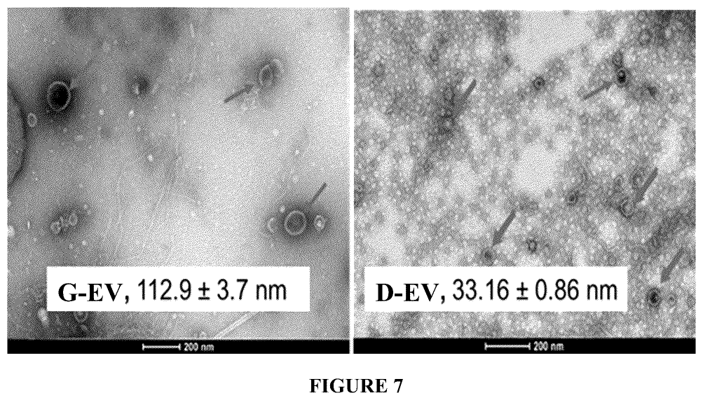

The shapes and sizes of the purified G-EVs and D-EVs were characterized using transmission electron microscopy (TEM) analysis (see Example 8 and FIG. 7). The size of EVs extracted during the exponential growth phase was larger (112.9±3.7 nm) than that of EVs extracted during the death/survival phase (33.2±0.9 nm). These results demonstrate that P. aeruginosa PAO1 biofilms release different populations of EVs at different stages, consistent with results observed for tumor cells and HeLa cells, which release different populations and subpopulations of EVs. This suggests that these distinct populations of EVs secreted by P. aeruginosa PAO1 biofilms at different stages have different functional roles in the biofilm life cycle.

### Example 4

### Biofilm-Derived EVs to Control Biofilm Formation and Development.

In this study, the research team reports that EVs secreted by P. aeruginosa PAO1 biofilms at different growth stages can have different effects on the same population of P. aeruginosa PAO1 biofilms, meaning that the functional effects of EVs are conditional, or environment dependent. For example, the growth of 2-h P. aeruginosa PAO1 biofilms can be enhanced by ˜2−log₁₀ CFU/cm² in the presence of G-EVs; however, the growth can be inhibited by the presence of their D-EVs (FIG. 3). These results also show that the presence of the D-EVs eliminated 8-hr. and 24-hr. P. aeruginosa PAO1 biofilms FIG. 3. G-EVs and D-EVs are both secreted by P. aeruginosa PAO1 biofilms, but at different biofilm growth stages. G-EVs are secreted in the exponential growth phase, while D-EVs are secreted in the survival/death phase. These results suggest that the functional roles of EVs are closely correlated with the functions of the parental cells under the specific conditions under which the EVs are secreted.

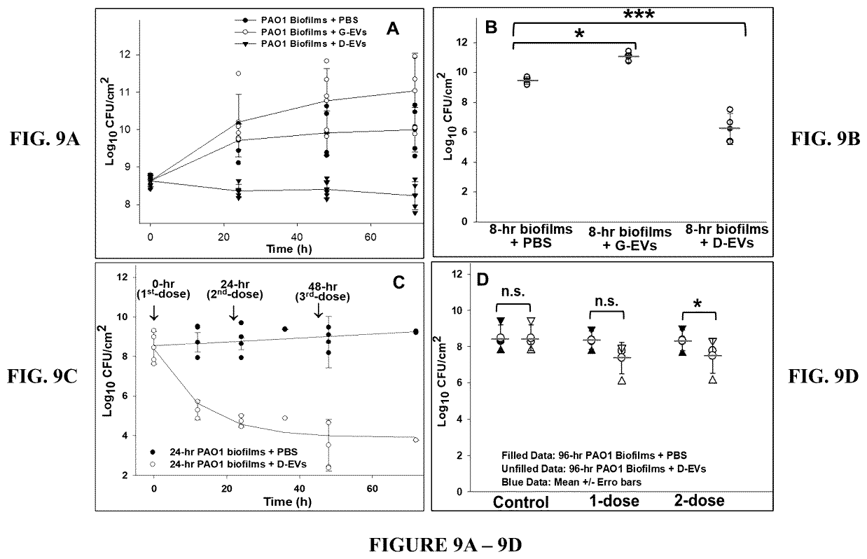

To investigate if EVs produced by a bacterial biofilm at a specific growth stage can influence the growth of recipient bacterial communities in the same developmental direction, the research team conducted a disc diffusion assay using both D-EVs and G-EVs to examine their functional effects on the growth behavior of P. aeruginosa PAO1 biofilm (see Example 8 and FIGS. 8A-8C). The team used PBS buffer and tetracycline as negative and positive controls, respectively, to assess the efficacy of D-EVs/G-EVs in inhibiting/promoting the formation and growth of P. aeruginosa PAO1 biofilms in comparison to tetracycline. Given that P. aeruginosa PAO1 is a known antibiotic-resistant superbug, the team used a sub-inhibitory concentration of tetracycline (1 μg/μL) in the test to ensure a clear zone on the dish (FIG. 9A). This concentration is much higher than the medically tolerated doses (0.033-0.22 μg/μL) used for adult admissions. Although the qualitative nature of the diffusion test made it difficult to discern the role of G-EVs in biofilm formation and growth, D-EVs exhibited an effective and dose-dependent inhibition of P. aeruginosa PAO1 biofilm growth across a range of extracellular vesicle protein concentrations (0.11, 0.22, and 0.33 μg/μL).

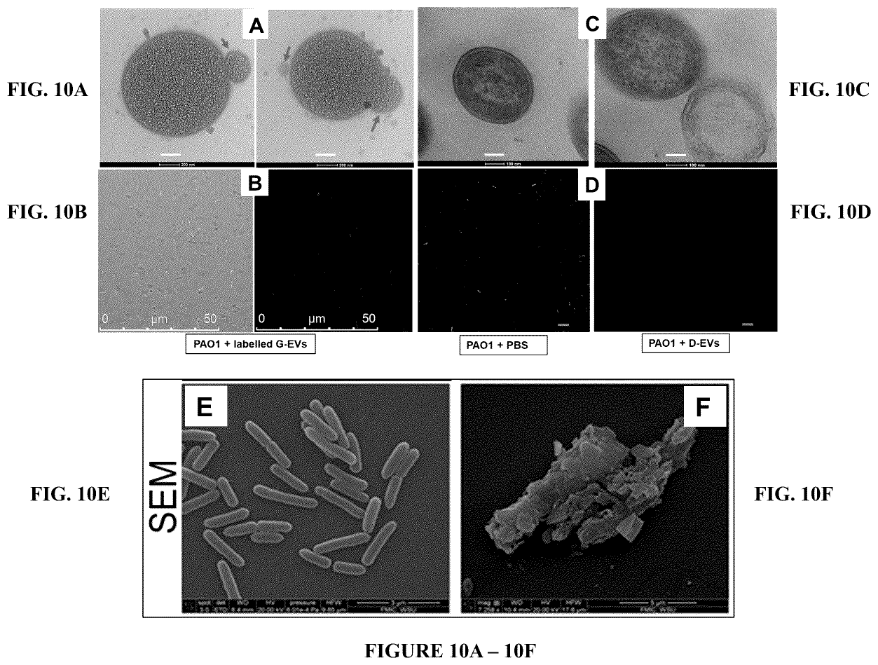

In order to confirm the importance of intact extracellular vesicle structure for the observed effects, the research team conducted an experiment in which the team disrupted the membranes of D-EVs and tested whether this affected their ability to inhibit biofilm growth. The team achieved this by subjecting the D-EV sample to water bath sonication, which lysed the EVs (see Example 8, below). The research team then used the sonicated D-EVs in the diffusion susceptibility test and compared the results to those obtained with intact D-EVs. The team found that the sonicated D-EVs had a much fainter inhibitory effect on biofilm growth than intact D-EVs (FIG. 10B), indicating that an intact EV structure is important for EVs to inhibit biofilm growth. These qualitative results are the first to demonstrate the potential of EVs secreted by a bacterial biofilm in its survival/death phase for use against bacterial biofilm growth of the same bacterium. D-EVs from Gram-positive S. aureus (MRSA) and Gram-negative E. coli showed effective cross-inhibition effects against PAO1 growths (FIG. 8C).

To assess how D-EVs and G-EVs affect biofilm growth, the research team monitored P. aeruginosa PAO1 biofilm growth in terms of Log₁₀ CFU/cm². The functional efficacy of the EVs varied depending on the maturity of the biofilm at each stage of development. FIG. 9A illustrates the growth of P. aeruginosa PAO1 bacterial biofilms grown for 2 hr. and treated with D-EVs and G-EVs compared to biofilms treated with PBS as a control. The results show that, compared to the control, the presence of G-EVs at a protein concentration of 0.028 μg/μL significantly increased the growth rate of the biofilms, while D-EVs at a protein concentration of 0.33 μg/μL showed a bactericidal effect on biofilm formation and growth. FIG. 9B shows the effects of D-EVs/G-EVs on the growth of P. aeruginosa PAO1 biofilms formed after 8 hours of growth (the exponential growth phase) after bacterial inoculation. It reveals a ˜2−log₁₀ increase in biofilm CFU counts after one dose of G-EVs at a protein content of 0.037 μg/μL. In contrast, treatment of the biofilm with one dose of D-EVs at a protein content of 0.33 μg/μL reduced CFU of biofilms ˜4.8−log₁₀ compared to the control. These experiments demonstrated that one-dose treatment of D-EVs/G-EVs significantly affected the behavior of biofilms in the initial lag and exponential growth phases (2-h and 8-h biofilms). The results from the D-EV experiments suggest feasibility of using D-EVs to treat P. aeruginosa PAO1 biofilms at the early stage of biofilm growth. These quantitative results provide direct evidence supporting the idea that G-EVs promote biofilm growth and D-EVs inhibit biofilm formation and growth.

FIG. 9C shows the inhibitory effects of multiple sequential doses of D-EVs at a protein concentration of 0.33 μg/μL on 24-hour biofilms at the plateau stage. CFU analysis of the treated biofilms 24 hours after the first dose showed a decrease in CFU of more than ˜3−log₁₀ compared to the control. After further doses at 24 hours and 48 hours, respectively, biofilm growth was inhibited by an additional ˜1.5−log₁₀. The observation of a total inhibition effect of 4.8−log₁₀ suggests that a multidose approach using D-EVs can be used to treat mature biofilms.

After reaching the survival/death phase, biofilms live in a cryptic growth mode and secrete D-EVs to coordinate bacterial functions and maintain viable cells by gradually recycling nutrients derived from dead cells. In this mode, biofilms age, and they are more difficult to eradicate and more resistant to environmental stresses, including antibiotics. The research team further investigated the effects of the extracted D-EVs on P. aeruginosa PAO1 biofilms (96-hr.) by applying either one dose of D-EVs at a protein concentration of 0.33 μg/μL directly to the aged biofilms or two doses separated by a 12-hr. interval. Analysis of the biomass collected 24 hours after the D-EV treatments is shown in FIG. 9D; only <1−log₁₀ reduction in P. aeruginosa PAO1 biofilms (96-hr.) was observed after 2 doses of D-EV treatment. The observed low inhibition effect was likely caused by the fact that the extracted D-EVs used for the treatment were secreted by P. aeruginosa PAO1 biofilms growing at the same developmental stage (after 96 hr. of growth).

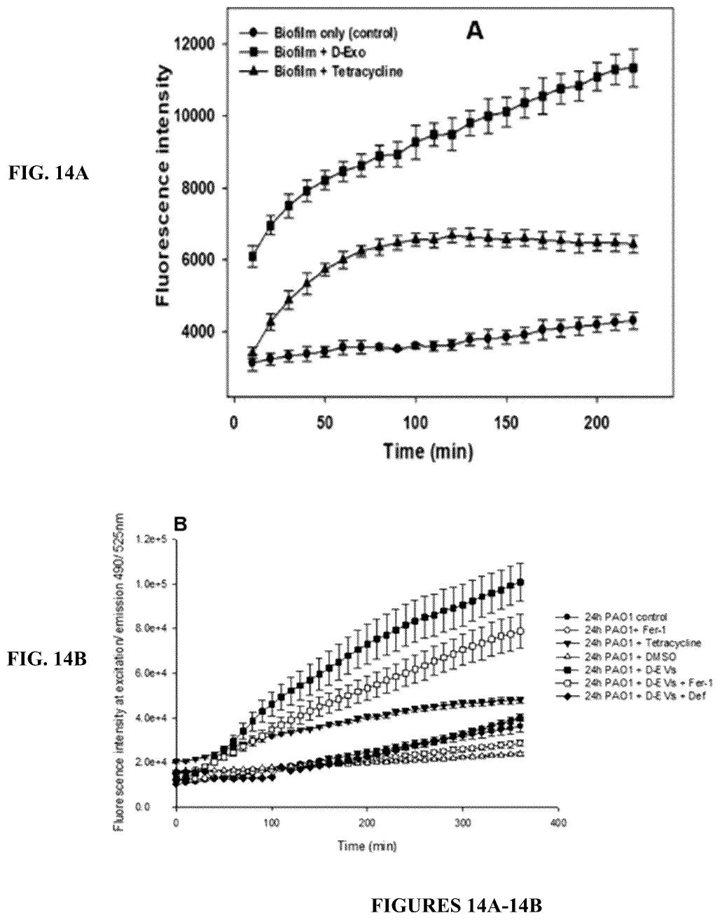

Interactions between the EVs and bacterial cells. To investigate whether the observed effects of D-EVs/G-EVs on P. aeruginosa PAO1 biofilm growth are due to direct interactions between the EVs and target cells, the team used TEM and CLSM to image potential interactions. The TEM images in FIG. 10A show that the G-EVs attached to the membrane surface of a target cell, triggering structural changes that led to EV uptake. In contrast, FIG. 10C shows that incubation with D-EVs led to deterioration of the bacterial cellular wall and subsequent cell death. These results are supported by CLSM experiments. The TEM images (FIG. 10A, 10C) show that D-EV treatment led to deterioration of the bacterial cellular wall and membranes, which subsequently led to cell death as shown in SEM images (FIGS. 10E, 10F). FIG. 10B shows that P. aeruginosa PAO1 cells became green fluorescent after being incubated with DAPI-labeled G-EVs, indicating uptake of the DAPI-labeled G-EVs by the bacteria, where DAPI specifically binds to A-T rich regions of the intra-vesical DNA. Confocal fluorescent images (FIG. 14) show individual PAO1 cells with blue-green fluorescence, suggesting that DAPI labeled G-EVs were up taken by PAO1 cells after incubation.

To image bacterial cell viability, the bacterial cells were positively stained with LIVE/DEAD BacLight Bacterial Viability Kits. FIG. 10D shows that most bacterial cells in control samples (no D-EV presence) were alive and emitting green fluorescence with a total corrected cell fluorescence of 2.5×10⁵±2.9×10³; however, after incubation with D-EVs, most of the cells were dead and emitting red fluorescence, indicating cell death; the green living cell total corrected fluorescence decreased to 3.1×10³±1.4×10². Overall, the finding that the G-EVs and D-EVs have different effects on bacterial cell structures may be directly related to the roles of G-EVs and D-EVs in promoting and inhibiting bacterial growth, respectively.

### Example 5

### Proteomic Analysis of the G-EVs and D-EVs.

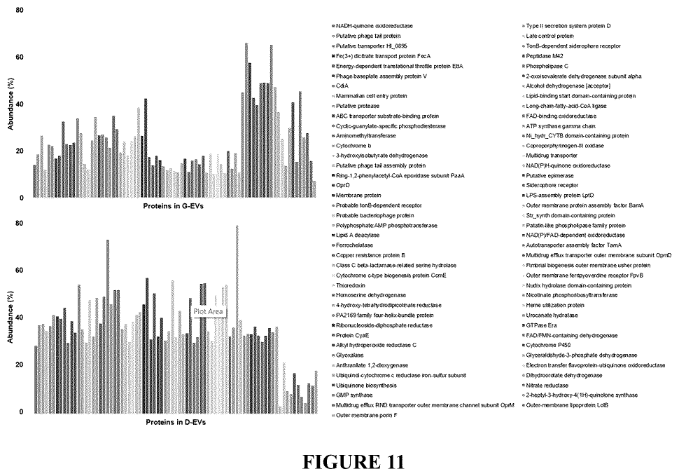

To further understand the different effects of D-EVs and G-EVs on PAO1 biofilm growth, proteomic profiles of the G-EVs and D-EVs were acquired. A total of 1099 and 987 proteins were found for G-EVs and D-EVs, respectively. Among these proteins, 79 surface and cytoplasmic proteins are shared by D-EVs and G-EVs with different abundances (FIG. 11). Additional proteomic profile analysis of G-EVs and D-EVs also identified a total of 92 (Table 2) and 77 (Table 3) highly abundant cytoplasmic proteins for the G EV and D-EV samples, respectively. The protein profiles of these two populations of bacterial EVs were found to be significantly different. The proteins carried by G-EVs were primarily associated with cell division and biosynthesis, DNA synthesis and protein processes. The proteins carried by D-EVs, such as D-amino acid oxidase (DAO) domain-containing protein, D-amino acid dehydrogenase, quinone oxidoreductase and 2,3-dihydro-3-hydroxy anthranilate isomerase, were mainly involved in inhibiting cell growth, reactive oxygen species (ROS) production and iron acquisition to promote cell survival or induce cell death.

Analysis also identified some key surface proteins that were uniquely present in different abundances on the surfaces of the D-EVs and G-EVs, which may contribute to the unique roles of D-EVs and G-EVs in biofilm growth. These proteins are listed in Table 2. Among them, two proteins (#1-#2) were exclusively present on the G-EV surface, while 13 (#3-#15) were exclusively present on the D-EV surface. Protein #1 on the G-EV surface is involved in the sulfate transport system of bacteria, which is responsible for energy coupling to the transport system, while protein #2 belongs to the CusCFBA copper efflux system, which plays a crucial role in copper homeostasis within the bacteria. With these important functional proteins on their surface, the G-EVs may enhance bacterial viability and promote cell growth under normal culture conditions through interactions with target bacterial cells.

TABLE 2
Profiles of key surface proteins on
G-EVs and D-EVs from PAO1 biofilms.

| No. | Proteins | Possible functions | G-EV | D-EV |
|---|---|---|---|---|
| 1 | Sulfate/thiosulfate | The sulfur-regulated gene | H | x |
| | import ATP-binding | (CysA) that encodes the | | |
| | protein CysA | membrane-associated ATP- | | |
| | | binding protein of the sulfate | | |
| | | transport system of bacteria is | | |
| | | responsible for energy | | |
| | | coupling to the transport | | |
| | | system. | | |
| 2 | HlyD_D23 domain- | The membrane fusion proteins | H | x |
| | containing protein | of the CusCFBA copper | | |
| | | efflux system that play a | | |
| | | crucial role in copper | | |
| | | homeostasis within the | | |
| | | bacteria, which is directly | | |
| | | associated with bacterial | | |
| | | viability. | | |
| 3 | Heme/hemoglobin | Promote acquisition of heme | x | H |
| | uptake outer | as iron resource for bacterial | | |
| | membrane receptor | growth. Because of its | | |
| | PhuR | capability of transferring | | |
| 4 | Hemin receptor | electrons at physiological pH, | x | H |
| 5 | Putative outer | iron plays a critical role in | x | H |
| | membrane ferric | bacterial physiology as an | | |
| | siderophore receptor | important component of | | |
| 6 | Putative | metabolic enzymes and | x | H |
| | hydroxamate-type | regulatory proteins. | | |
| | ferrisiderophore | | | |
| | receptor | | | |
| 7 | Heme acquisition | | x | H |
| | protein HasAp | | | |
| 8 | Ferripyoverdine | | x | H |
| | receptor | | | |
| 9 | Extracellular heme- | | x | H |
| | binding protein | | | |
| 10 | TonB-dependent | Either mediates the release of | x | H |
| | receptor | iron into the periplasm of the | | |
| 11 | Putative TonB- | bacteria or transports ferric | x | H |
| | dependent receptor | enterobactin into the | | |
| 12 | Ferric enterobactin | periplasm. | x | H |
| | receptor | | | |
| 13 | AMP-binding | Regulatory enzyme in | x | H |
| | protein | response to environmental | | |
| | | changes. | | |
| 14 | Quinone | Reduces quinones into | x | H |
| | oxidoreductase-like | hydroquinone, without proton | | |
| | protein 2 | translocation. | | |
| 15 | 2,3-dihydro-3- | Phenazine-1-carboxylate | x | H |
| | hydroxyanthranilate | biosynthesis/important for | | |
| | isomerase | redox cycling. | | |

Note:
H—high abundancy; x—not found. n = 3.

The thirteen D-EV surface proteins can be categorized into three groups. A selection of D-EV surface proteins (#3-#9) are membrane receptors or functional proteins that promote bacterial growth by enhancing the acquisition of iron resources. An additional grouping (#10-#12) includes membrane receptors that transport ferric enterobactin into the periplasm or release iron into the periplasm of the bacteria. Iron is an important component of metabolic enzymes and regulatory proteins that support the growth and survival of most bacterial species. However, excessive uptake of iron by bacteria can be detrimental because of the iron-triggered Fenton/Haber-Weiss reaction, which produces harmful ROS such as superoxide (O²⁻), hydrogen peroxide (H₂O₂), and the highly destructive hydroxyl radical (OH). Accumulation of ROS can inactivate key functional enzymes and activate bacterial programmed cell death. Another group of D-EV surface proteins (#14-#15) includes quinone oxidoreductase and 2,3-dihydro-3-hydroxyanthranilate isomerase, which participate directly in the Fenton reaction and ROS generation. Of the unique D-EV proteins, more than half are associated with cellular functions that regulate H₂O₂ generation or enhance cellular acquisition of iron.

Additional proteins identified, A0A1C7BCH7, GSH-dependent formaldehyde dehydrogenase, and A0A0A8RFF77, Quinione oxidoreductase-like protein 2 have been shown to be involved in regulating the intracellular redox ratio of GSH/GSSG and the phenazine/pyocyanin redox cycle in such a way as to shift the redox balance toward a more oxidative stress state within bacterial cells, thus creating an intracellular environment more favorable to intracellular ROS production.

A group of group of acyl-homoserine lactone (AHL) acylase proteins, including, Q9I4U2, AHL acylase QuiP, is also found exclusively in D-EVs. AHLs are important signaling molecules in bacterial quorum sensing processes. These enzymes specifically target and degrade AHLs, thus quenching quorum sensing and affecting biofilm growth. This is corroborated by the observation that the abundance of the enzyme in the proteomic profile of untreated PAO1 biofilms at the exponential growing stage is significantly increased after treatment with D-EVs.

TABLE 3
Summary of proteomic profiles of cytoplasmic proteins of G-EVs.

| | | Sum | | # | # | | | |
|---|---|---|---|---|---|---|---|---|
| | | PEP | Coverage | of | Unique | # | MW | |
| Accession | Description | Score | [%] | PSMs | Peptides | AAs | [kDa] | Abundances |
| Growth promotors | | | | | | | | |
| A0A653B731 | Cell division proteins | 6.093 | 4 | 1 | 1 | 398 | 41.7 | 100 |
| V6A9F4 | Phasin_2 domain- | 1.275 | 12 | 2 | 1 | 153 | 16.9 | 34.64 |
| | containing protein | | | | | | | |
| A0A1C7B9B8 | Elongation factors | 48.011 | 23 | 22 | 11 | 706 | 77.7 | 58.34 |
| A0A7W3UUK1 | Chaperonins | 11.74 | 10 | 7 | 1 | 527 | 55.7 | 32.5 |
| A0A3D9EJG5 | Chaperone(s) | 1.443 | 4 | 1 | 1 | 433 | 47.9 | 35.28 |
| DNA synthesis | | | | | | | | |
| A0A072ZFF8 | N5-carboxyaminoimidazole | 37.121 | 67 | 22 | 5 | 163 | 16.9 | 60.32 |
| | ribonucleotide mutase | | | | | | | |
| A0A086BUQ2 | Vitamin B12-dependent | 14.186 | 7 | 4 | 3 | 734 | 82.7 | 64.4 |
| | ribonucleotide reductase | | | | | | | |
| S6ABT0 | Ribonucleotide reductase | 3.464 | 3 | 3 | 1 | 324 | 35.6 | 33.98 |
| A0A3M5EFC0 | Polyribonucleotide | 116.971 | 34 | 60 | 16 | 748 | 80.3 | 49.36 |
| | nucleotidyltransferase | | | | | | | |
| A0A367M4C8 | HU family DNA-binding | 27.865 | 49 | 19 | 5 | 93 | 9.8 | 68.58 |
| | protein | | | | | | | |
| A0A069PX19 | DNA polymerases | 18.819 | 11 | 7 | 6 | 913 | 99.7 | 61.2 |
| A0A086BZZ8 | DNA topoisomerase | 13.875 | 10 | 6 | 5 | 868 | 97.2 | 56.28 |
| Q913X2 | DNA helicase | 7.812 | 6 | 3 | 3 | 711 | 79.8 | 41.14 |
| A0A485FIC3 | DNA gyrase | 41.307 | 11 | 18 | 6 | 925 | 101.3 | 43 |
| A0A2S5IJE8 | Holliday junction ATP- | 1.637 | 6 | 1 | 1 | 205 | 22.3 | 69.12 |
| | dependent DNA helicase | | | | | | | |
| Protein processing | | | | | | | | |
| A0A086C2F0 | Peptide-binding protein | 9.811 | 18 | 3 | 3 | 222 | 24.1 | 100 |
| A0A397MC94 | SsrA-binding protein | 2.281 | 6 | 1 | 1 | 197 | 22.3 | 100 |
| A4XZJ8 | Putative serine protein | 14.827 | 11 | 9 | 1 | 640 | 73.7 | 100 |
| | kinase | | | | | | | |
| A0A485EER1 | NAD-capped RNA | 3.84 | 2 | 1 | 1 | 841 | 92.3 | 100 |
| | hydrolase | | | | | | | |
| A0A367M033 | 2-oxo-4-hydroxy-4- | 3.647 | 9 | 1 | 1 | 178 | 19.6 | 100 |
| | carboxy-5- | | | | | | | |
| | ureidoimidazoline | | | | | | | |
| | decarboxylase | | | | | | | |
| A0A080VRC4 | S- | 3.558 | 4 | 1 | 1 | 347 | 38.1 | 100 |
| | adenosylmethionine:tRNA | | | | | | | |
| | ribosyltransferase- | | | | | | | |
| | isomerase | | | | | | | |
| A0A5E9K219 | Ribosomal RNA large | 2.577 | 12 | 2 | 1 | 155 | 17.8 | 100 |
| | subunit methyltransferase H | | | | | | | |
| A0A072ZEM5 | Aminopeptidase P family | 2.244 | 3 | 1 | 1 | 405 | 44.1 | 100 |
| | protein | | | | | | | |
| A0A0H2ZCX7 | Putative MoxR protein O | 2.446 | 4 | 1 | 1 | 305 | 32.8 | 100 |
| A0A1I1XAM6 | Adenylyltransferase and | 1.941 | 4 | 1 | 1 | 270 | 28.4 | 100 |
| | sulfurtransferase | | | | | | | |
| W1MVH1 | Glutathione S-transferase | 1.38 | 5 | 1 | 1 | 256 | 28.8 | 100 |
| A0A0A8RCC8 | Periplasmic tail-specific | 2.185 | 1 | 1 | 1 | 840 | 93.5 | 100 |
| | protease | | | | | | | |
| A0A072ZJB8 | Carboxy-terminal | 2.716 | 8 | 2 | 2 | 436 | 46 | 31.7 |
| | processing protease precurs | | | | | | | |
| B7V669 | 50S ribosomal proteins | 56.677 | 49 | 26 | 7 | 129 | 14.5 | 42.56 |
| A0A1C7C943 | 30S ribosomal proteins | 35.017 | 28 | 26 | 4 | 246 | 27.3 | 33.62 |
| A0A231K3D3 | Chain-length determining | 16.217 | 17 | 5 | 4 | 442 | 49.2 | 8.08 |
| | protein | | | | | | | |
| W1MWV0 | Glycine--tRNA ligase alpha | 13.843 | 24 | 7 | 5 | 318 | 36.5 | 42.18 |
| | subunit | | | | | | | |
| A0A0C7D284 | Tyrosine--tRNA ligase | 6.676 | 10 | 2 | 2 | 399 | 44.1 | 80.42 |
| A0A0A8RE97 | Leucine--tRNA ligase | 6.149 | 3 | 4 | 2 | 873 | 97.6 | 30.8 |
| Q9HXU0 | Lysine--tRNA ligase | 11.043 | 12 | 4 | 4 | 501 | 57.3 | 72.02 |
| A0A0F6RS41 | Phenylalanine--tRNA ligase | 1.284 | 2 | 1 | 1 | 792 | 86.7 | 61.18 |
| A0A367MBX4 | Glutamine--tRNA ligase | 7.116 | 8 | 4 | 3 | 561 | 63.3 | 83.14 |
| L8MK56 | Glutamyl-tRNA(Gln) | 4.254 | 6 | 1 | 1 | 483 | 51.4 | 58.26 |
| | amidotransferase subunit | | | | | | | |
| A0A2R4BHU9 | Aspartyl/glutamyl- | 3.26 | 3 | 3 | 1 | 492 | 54.6 | 50.4 |
| | tRNA(Asn/Gln) | | | | | | | |
| | amidotransferase subunit | | | | | | | |
| A0A485HEP7 | D-aminoacyl-tRNA | 3.839 | 4 | 1 | 1 | 468 | 51.9 | 86.76 |
| | deacylase | | | | | | | |
| A0A1H0JDB1 | Succinate--CoA ligase | 25.501 | 17 | 9 | 6 | 388 | 41.5 | 68.46 |
| A0A0A8RKE2 | Isoleucine--tRNA ligase | 42.217 | 14 | 23 | 9 | 943 | 105.4 | 52.02 |
| A6V3C2 | Ribonuclease | 11.6 | 5 | 4 | 3 | 1073 | 119.3 | 49.66 |
| A0A086C201 | RNA polymerase | 8.959 | 6 | 5 | 1 | 620 | 70.1 | 53.84 |
| A0A087L9F7 | Alkaline phosphatase | 7.665 | 12 | 3 | 2 | 269 | 30 | 59.9 |
| | family protein | | | | | | | |
| A0A2R3ISX8 | Bifunctional proteins | 6.982 | 13 | 3 | 3 | 454 | 48.8 | 83.18 |
| A0A1G8LM51 | RNA-binding protein | 23.925 | 66 | 18 | 6 | 86 | 9.5 | 43.04 |
| A0A0A8RIM8 | Chitin-binding protein | 17.973 | 28 | 12 | 6 | 389 | 41.8 | 46.38 |
| A0A0D6INW2 | Ribosome-binding factor | 10.607 | 32 | 4 | 3 | 130 | 14.7 | 56.04 |
| A0A127MQF6 | Ribosome modulation | 5.574 | 21 | 3 | 1 | 72 | 8.4 | 46.14 |
| | factor | | | | | | | |
| A0A2R3INC8 | Ribosome-recycling factor | 15.315 | 22 | 10 | 3 | 185 | 20.5 | 86.18 |
| A0A1C7BK43 | Periplasmic serine | 1.523 | 3 | 1 | 1 | 474 | 50.3 | 65.86 |
| | endoprotease | | | | | | | |
| A0A3M5DFV1 | ATP-dependent Clp | 13.897 | 8 | 7 | 2 | 452 | 50.1 | 67.32 |
| | protease ATP-binding | | | | | | | |
| | subunit | | | | | | | |
| A0A069PZV2 | Enoyl-[acyl-carrier-protein] | 1.652 | 6 | 1 | 1 | 265 | 28 | 73.34 |
| | reductase [NADH] | | | | | | | |
| K7Y4J0 | Alkaline metalloprotease | 9.962 | 7 | 5 | 2 | 481 | 50.6 | 59.52 |
| A0A072ZPJ9 | Metalloprotease | 7.361 | 6 | 2 | 2 | 449 | 47.8 | 70.46 |
| A6VCK8 | ATP-dependent zinc | 6.179 | 2 | 2 | 1 | 642 | 70.3 | 43.9 |
| | metalloprotease | | | | | | | |
| A0A0A8RQS8 | Zn_protease domain- | 10.585 | 20 | 7 | 3 | 221 | 24.3 | 67.76 |
| | containing protein | | | | | | | |
| A0A1I1UAD0 | Ferritin-like metal-binding | 5.342 | 8 | 3 | 1 | 169 | 18.8 | 63.86 |
| | proteins | | | | | | | |
| Flagella and Pilli | | | | | | | | |
| A0A071KYA9 | Flagellar biosynthesis | 6.771 | 17 | 2 | 2 | 156 | 17.2 | 88.96 |
| | protein | | | | | | | |
| A0A0A8RCK3 | Protein pilG | 4.435 | 13 | 1 | 1 | 135 | 14.7 | 80.94 |
| A0A379IZN6 | Type IV-A pilus assembly | 1.889 | 4 | 2 | 1 | 581 | 63.8 | 72.98 |
| | ATPase | | | | | | | |
| Q9HVM8 | Type IV pilus biogenesis | 56.48 | 22 | 23 | 12 | 1161 | 126.5 | 33.6 |
| | factor PilY1 | | | | | | | |
| A0A0A8RBR3 | Protein pilJ | 20.55 | 9 | 10 | 4 | 682 | 72.5 | 30.84 |
| Synthases | | | | | | | | |
| A0A3M5EVT4 | 3-dehydroquinate synthase | 5.761 | 9 | 2 | 2 | 421 | 46.1 | 100 |
| | OS = Pseudomonas | | | | | | | |
| | aeruginosa OX = 287 | | | | | | | |
| | GN = aroB PE = 3 SV = 1 | | | | | | | |
| A0A069Q8Q1 | 1,4-Dihydroxy-2- | 5.632 | 7 | 1 | 1 | 265 | 28.9 | 100 |
| | naphthoyl-CoA synthase | | | | | | | |
| | OS = Pseudomonas | | | | | | | |
| | aeruginosa OX = 287 | | | | | | | |
| | GN = menB_1 PE = 3 | | | | | | | |
| | SV = 1 | | | | | | | |
| A0A291KBQ0 | S-adenosylmethionine | 5.509 | 7 | 2 | 1 | 396 | 42.7 | 100 |
| | synthase OS = Pseudomonas | | | | | | | |
| | mendocina OX = 300 | | | | | | | |
| | GN = metK PE = 3 SV = 1 | | | | | | | |
| A0A0A8RA44 | Acetolactate synthase | 4.099 | 3 | 2 | 1 | 592 | 64.7 | 100 |
| | OS = Pseudomonas | | | | | | | |
| | aeruginosa OX = 287 | | | | | | | |
| | GN = PAMH19_0813 | | | | | | | |
| | PE = 3 SV = 1 | | | | | | | |
| A0A0A8RC77 | Anthranilate synthase | 2.933 | 4 | 1 | 1 | 496 | 55.1 | 100 |
| | component 1 | | | | | | | |
| | OS = Pseudomonas | | | | | | | |
| | aeruginosa OX = 287 | | | | | | | |
| | GN = trpE PE = 3 SV = 1 | | | | | | | |
| A0A3D9EI68 | ATP synthase | 130.604 | 42 | 77 | 1 | 458 | 49.7 | 37.78 |
| A0A0A8RS72 | Hydrogen cyanide synthase | 33.235 | 29 | 10 | 8 | 464 | 50.4 | 83.46 |
| A0A077JN20 | GMP synthase | 9.213 | 8 | 5 | 3 | 527 | 58.1 | 45.4 |
| A0A0A8RJH1 | Glucans biosynthesis | 8.951 | 7 | 4 | 4 | 861 | 97 | 91.06 |
| | glucosyltransferase | | | | | | | |
| A0A127MNP8 | Argininosuccinate synthase | 8.785 | 5 | 4 | 2 | 405 | 45.4 | 73.16 |
| A0A0F6UIJ7 | Carbamoyl-phosphate | 8.595 | 3 | 3 | 2 | 1073 | 117.3 | 66.24 |
| | synthase large chain | | | | | | | |
| A0A081HI22 | Tryptophan synthase | 8.141 | 17 | 2 | 2 | 268 | 28.5 | 84.78 |
| A0A0A8RJ65 | PQB biosynthetic 3- | 7.385 | 7 | 5 | 2 | 348 | 37.6 | 69.48 |
| | oxoacyl-[acyl-carrier- | | | | | | | |
| | protein] synthase | | | | | | | |
| A0A5K1SNI7 | Chorismate synthase | 5.676 | 8 | 2 | 2 | 363 | 38.9 | 33.44 |
| A0A127MLM1 | Thiazole synthase | 5.568 | 11 | 3 | 2 | 268 | 28.5 | 42.72 |
| A0A6H3G8H9 | Lipopolysaccharide | 5.095 | 7 | 3 | 2 | 662 | 74.5 | 64.84 |
| | biosynthesis protein | | | | | | | |
| A0A0A8RHR0 | Acetolactate synthase | 4.525 | 3 | 2 | 1 | 574 | 63 | 90.44 |
| A0A0A8RG31 | Dihydrofolate | 4.132 | 4 | 2 | 1 | 429 | 46.5 | 61.64 |
| | synthase/folylpolyglutamate | | | | | | | |
| | synthase | | | | | | | |
| A0A3M5DAV0 | Biotin synthase | 1.038 | 3 | 1 | 1 | 390 | 43.3 | 85.78 |
| A0A3M5ENA3 | Citrate synthase | 96.305 | 62 | 53 | 14 | 429 | 47.8 | 51.02 |
| A0A0A8RPU8 | Phosphoenolpyruvate | 55.493 | 22 | 34 | 13 | 791 | 85.8 | 44.82 |
| | synthase | | | | | | | |
| A0A0C6EL94 | Malate synthase | 45.881 | 29 | 17 | 9 | 725 | 78.6 | 55.46 |
| A0A1C7BDZ1 | Pyridoxine 5′-phosphate | 29.474 | 27 | 18 | 5 | 248 | 27.2 | 44.54 |
| | synthase | | | | | | | |
| A0A086BTL8 | Adenylosuccinate | 29.298 | 27 | 16 | 7 | 430 | 46.8 | 45.76 |
| | synthetase | | | | | | | |
| A0A069QI07 | Glutathione synthetase | 21.722 | 25 | 8 | 4 | 317 | 35.7 | 34.52 |
| Oxidoreductase enzymes | | | | | | | | |
| A0A5F1BV16 | Re/Si-specific NAD(P)(+) | 8.376 | 10 | 3 | 2 | 373 | 38.8 | 100 |
| | transhydrogenase | | | | | | | |
| A0A0H2Z7C1 | N-succinylglutamate 5- | 5.799 | 4 | 1 | 1 | 488 | 51.5 | 100 |
| | semialdehyde | | | | | | | |
| | dehydrogenase | | | | | | | |
| A0A485EKG2 | Gluconate dehydrogenase | 5.047 | 3 | 3 | 2 | 1275 | 138.2 | 100 |
| A0A7U9F023 | GDP-mannose 6- | 2.742 | 3 | 1 | 1 | 442 | 48.2 | 100 |
| | dehydrogenase | | | | | | | |

Notes for Table 3:
# PSMs (the total number of identified peptide spectramatched for the protein);
FDR Confidence Combined was high for all proteins;
Exp. q-value: Combined was between 0-0.004.

TABLE 4
Summary of proteomic profiles of cytoplasmic proteins of D-EVs.

| | | Sum | | | # | | | |
|---|---|---|---|---|---|---|---|---|
| | | PEP | Coverage | # | Unique | # | MW | Abundance |
| Accession | Description | Score | [%] | PSMs | Peptides | AAs | [kDa] | (%) |
| Fatty acid oxidation and hydrogen | | | | | | | | |
| peroxide production | | | | | | | | |
| A0A1C7BKA1 | DAO domain-containing | 6.348 | 6 | 2 | 2 | 468 | 52.2 | 44.44 |
| | protein | | | | | | | |
| A0A2R3QM54 | D-amino acid dehydrogenase | 3.474 | 10 | 3 | 1 | 432 | 47 | 100 |
| A0A127MPZ7 | Soluble pyridine nucleotide | 112.322 | 57 | 94 | 1 | 464 | 51.3 | 72.72 |
| | transhydrogenase | | | | | | | |
| A0A1H0H0K0 | 2-hydroxycyclo- | 1.326 | 4 | 2 | 1 | 255 | 26.1 | 67.18 |
| | hexanecarboxyl- | | | | | | | |
| | CoA dehydrogenase | | | | | | | |
| A0A080VW75 | CatB-related O- | 1.726 | 7 | 1 | 1 | 229 | 25.6 | 65.42 |
| | acetyltransferase | | | | | | | |
| A0A069QJT4 | Glycerophosphodiester | 23.456 | 26 | 10 | 4 | 240 | 26.9 | 31.98 |
| | phosphodiesterase | | | | | | | |
| A0A080VGG0 | Acetyl-coenzyme A | 9.765 | 5 | 3 | 2 | 645 | 71.6 | 35.4 |
| | synthetase | | | | | | | |
| A0A1G8E7R7 | Glutamate synthase | 4.786 | 1 | 1 | 1 | 1482 | 161.9 | 54.66 |
| | (NADPH/NADH) large | | | | | | | |
| | chain | | | | | | | |
| A0A0A8RMZ0 | Dihydrolipoamide | 23.135 | 6 | 10 | 2 | 428 | 45.5 | 42.34 |
| | acetyltransferase component | | | | | | | |
| | of pyruvate dehydrogenase | | | | | | | |
| | complex | | | | | | | |
| Protein oxidation and activate the Fenton | | | | | | | | |
| reaction | | | | | | | | |
| A0A7U0JSC5 | AAA family ATPase | 6.038 | 5 | 3 | 2 | 555 | 57.7 | 80 |
| A0A3M5EPG2 | Dihydroxy-acid dehydratase | 2.593 | 2 | 2 | 1 | 680 | 72.7 | 39.26 |
| A0A3M5DZ62 | OMP_b-brl domain- | 60.174 | 42 | 27 | 10 | 236 | 25.6 | 53.68 |
| | containing protein | | | | | | | |
| A0A3M5D221 | STN domain-containing | 5.527 | 2 | 2 | 1 | 977 | 105.6 | 71.16 |
| | protein | | | | | | | |
| A0A1H0IZF5 | LPS-assembly protein LptD | 1.195 | 1 | 1 | 1 | 912 | 102.8 | 57.06 |
| A0A3D9E7A8 | Urease accessory protein | 1.136 | 10 | 1 | 1 | 223 | 24.4 | 77.22 |
| | UreF | | | | | | | |
| A0A5F1BVL6 | YkgJ family cysteine cluster | 1.281 | 15 | 1 | 1 | 223 | 24.8 | 81.88 |
| | protein | | | | | | | |
| A0A0A8RCJ6 | UvrABC system protein A | 13.698 | 7 | 5 | 5 | 1003 | 110.3 | 42.78 |
| A0A1G9YY22 | Sterol carrier protein | 6.833 | 16 | 1 | 1 | 104 | 11.1 | 100 |
| A0A0A8RGJ4 | Protein PelC | 7.15 | 12 | 2 | 1 | 172 | 18.6 | 72.7 |
| A0A072ZPD2 | Amino-acid carrier protein | 10.903 | 6 | 2 | 1 | 449 | 47.3 | 31.46 |
| | AlsT | | | | | | | |
| W1MFI8 | Lactamase_B domain- | 8.994 | 7 | 5 | 2 | 433 | 48.6 | 41.32 |
| | containing protein | | | | | | | |
| A0A0A8RHK2 | MaoC-like domain- | 10.152 | 19 | 4 | 3 | 285 | 31.1 | 39.32 |
| | containing protein | | | | | | | |
| A0A080VTX5 | Putative periplasmic | 8.078 | 11 | 4 | 2 | 319 | 33.6 | 64.1 |
| | transport protein | | | | | | | |
| A0A086C084 | Terminase OS = | 3.309 | 9 | 1 | 1 | 119 | 13.3 | 30.72 |
| | Pseudomonas aeruginosa | | | | | | | |
| | VRFPA01 | | | | | | | |
| Unfold DNA, RNA, and proteins | | | | | | | | |
| A0A0A8RE90 | ATP-dependent RNA | 3.444 | 2 | 1 | 1 | 579 | 63.8 | 65 |
| | helicase RhlB | | | | | | | |
| A0A072ZF84 | 3-guanidinopropionase | 5.869 | 6 | 2 | 1 | 318 | 34.2 | 57.04 |
| Promote cellular adhesion | | | | | | | | |
| A0A367LV53 | Aldehyde dehydrogenase | 1.788 | 11 | 2 | 1 | 99 | 11 | 100 |
| | family protein (Fragment) | | | | | | | |
| A0A2R3IQ67 | Neisseria PilC beta-propeller | 6.646 | 3 | 2 | 1 | 1155 | 126.1 | 100 |
| | domain protein | | | | | | | |
| A0A1I1TGJ6 | Flagellar L-ring protein | 3.893 | 8 | 1 | 1 | 237 | 24.9 | 100 |
| A0A485GA02 | Type 4 fimbrial biogenesis | 11.1 | 11 | 4 | 3 | 625 | 68.7 | 30.4 |
| | protein PilW | | | | | | | |
| A0A0F6UFV5 | Flagellar hook-associated | 121.787 | 60 | 63 | 16 | 439 | 46.8 | 47.68 |
| | protein 3 | | | | | | | |
| A0A1C7BE42 | Fimbrial assembly protein | 33.737 | 21 | 22 | 9 | 714 | 77.4 | 68.1 |
| | pilQ | | | | | | | |
| A0A022P6S5 | Flagellar basal-body rod | 31.805 | 39 | 17 | 7 | 261 | 27.7 | 40.98 |
| | protein FlgG | | | | | | | |
| A0A072ZBU1 | Fap amyloid fiber secretin | 5.721 | 8 | 3 | 2 | 421 | 45.7 | 40.02 |
| A0A3M5EU50 | Flagellar P-ring protein | 6.05 | 6 | 3 | 2 | 554 | 57.4 | 90.62 |
| Virulence factors | | | | | | | | |
| A0A7Z0KFT4 | Flagellin | 6.102 | 12 | 5 | 1 | 397 | 40.6 | 100 |
| A0A6M5KA93 | B-type flagellin | 74.411 | 84 | 74 | 7 | 126 | 13.3 | 92.02 |
| Catalyzes | | | | | | | | |
| A0A0A8R9J6 | Hydrolase_4 domain- | 4.573 | 7 | 2 | 2 | 339 | 38.3 | 100 |
| | containing protein | | | | | | | |
| A0A072ZRC9 | Nucleotide sugar | 2.571 | 2 | 1 | 1 | 665 | 74.3 | 100 |
| | epimerase/dehydratase | | | | | | | |
| | WbpM | | | | | | | |
| A0A5E5R2E7 | Esterase EstA | 116.039 | 43 | 74 | 2 | 646 | 69.6 | 74.84 |
| A0A7U9F3D4 | Transaldolase | 5.763 | 7 | 3 | 1 | 309 | 33.9 | 34.6 |
| A0A7U4A4H5 | Soluble pyridine nucleotide | 170.703 | 63 | 138 | 5 | 464 | 51.1 | 66.72 |
| | transhydrogenase | | | | | | | |
| V6AM38 | Carbamoyl-phosphate | 2.158 | 3 | 1 | 1 | 398 | 43.2 | 100 |
| | synthase small chain | | | | | | | |
| Q9L6C7 | Triacylglycerol acylhydrolase | 137.528 | 59 | 114 | 3 | 311 | 32.7 | 41.98 |
| A0A0H2ZGA2 | RND efflux membrane fusion | 40.982 | 31 | 19 | 7 | 370 | 39.1 | 51.02 |
| | protein | | | | | | | |
| A0A1C7B566 | Lysin domain-containing | 40.492 | 21 | 22 | 7 | 341 | 37.6 | 75.92 |
| | protein | | | | | | | |
| A0A077JXU0 | Alpha/beta hydrolase | 1.343 | 4 | 2 | 1 | 275 | 30.4 | 46.26 |
| A0A643IQV2 | Protocatechuate 3,4- | 7.402 | 10 | 1 | 1 | 239 | 27.2 | 100 |
| | dioxygenase subunit beta | | | | | | | |
| A0A0A8RRW3 | Uricase | 3.874 | 6 | 2 | 2 | 494 | 55 | 57.36 |
| A0A485EQT2 | Signal peptidase I | 6.5 | 16 | 2 | 2 | 214 | 24 | 40.88 |
| A0A1C7BZ49 | Lipoprotein | 6.93 | 7 | 5 | 1 | 210 | 23.7 | 56.8 |
| A0A086BXD4 | Peptidyl-tRNA hydrolase | 1.248 | 7 | 1 | 1 | 194 | 20.8 | 100 |
| Cellular permeability | | | | | | | | |
| A0A086BU08 | ABC transporter permease | 1.279 | 3 | 2 | 1 | 231 | 24.9 | 54.84 |
| A0A3S0IYB5 | Autotransporter domain- | 104.446 | 21 | 83 | 1 | 991 | 104.4 | 41.58 |
| | containing protein | | | | | | | |
| A0A0H2Z999 | Putative outer membrane | 51.845 | 24 | 19 | 11 | 705 | 79.2 | 33.32 |
| | receptor protein | | | | | | | |
| Q9HVG7 | POTRA domain-containing | 101.304 | 54 | 57 | 18 | 568 | 63.2 | 37.78 |
| | proteinX = 208964 | | | | | | | |
| | GN = PA4624 PE = 3 | | | | | | | |
| | SV = 1 | | | | | | | |
| A0A7M3A5L5 | Porins | 71.452 | 59 | 47 | 1 | 204 | 22.9 | 100 |
| A0A0A8RIR9 | ABC transporter ATP- | 2.707 | 2 | 1 | 1 | 682 | 74.7 | 100 |
| | binding protein/permease | | | | | | | |
| A0A6N4IRW4 | Type VI secretion system | 23.3 | 18 | 11 | 5 | 491 | 55.5 | 59.14 |
| | contractile sheath large | | | | | | | |
| | subunit | | | | | | | |
| A0A3M5E7S2 | ATP-grasp domain- | 23.138 | 21 | 9 | 7 | 519 | 59.5 | 64.08 |
| | containing protein | | | | | | | |
| A0A072ZK48 | Outer membrane lipoprotein | 1.537 | 5 | 1 | 1 | 189 | 22 | 14.24 |
| | Blc | | | | | | | |
| A0A080VSF7 | Pseudopaline transport outer | 11.172 | 8 | 3 | 3 | 708 | 79 | 55.96 |
| | membrane protein CntO | | | | | | | |
| Regulators | | | | | | | | |
| A0A6A9JUZ1 | Response regulator | 3.509 | 9 | 1 | 1 | 212 | 23.2 | 100 |
| A0A1H0PGG6 | Soluble pyridine nucleotide | 77.621 | 33 | 68 | 2 | 464 | 51.1 | 85.7 |
| | transhydrogenase | | | | | | | |
| A0A7Y9XNH5 | Type I restriction enzyme R | 1.097 | 3 | 1 | 1 | 910 | 103.1 | 100 |
| | subunit | | | | | | | |
| A0A5R1AUH1 | TetR family transcriptional | 2.007 | 4 | 1 | 1 | 212 | 24 | 39.9 |
| | regulator | | | | | | | |
| A0A069QCL6 | Protocatechuate 3,4- | 2.443 | 7 | 2 | 1 | 201 | 22.8 | 53.08 |
| | dioxygenase alpha chain | | | | | | | |
| A0A086BV97 | Transcriptional regulator | 2.769 | 5 | 1 | 1 | 225 | 25.5 | 56.6 |
| V6AMV0 | Putative methyl-accepting | 13.017 | 3 | 4 | 1 | 859 | 91.1 | 63 |
| | chemotaxis protein | | | | | | | |
| A0A0A8RAB2 | Putative HTH-type | 14.153 | 11 | 6 | 4 | 500 | 55.3 | 100 |
| | transcriptional regulator | | | | | | | |
| | YdcR | | | | | | | |
| A0A0D7MRK9 | Chemotaxis transducer | 16.779 | 12 | 4 | 3 | 545 | 58.3 | 71.76 |
| A0A0A8RJ29 | HIT domain-containing | 4.547 | 7 | 1 | 1 | 209 | 23 | 69.86 |
| | protein | | | | | | | |
| A0A3M5ED98 | HTH tetR-type domain- | 3.801 | 4 | 1 | 1 | 274 | 30 | 100 |
| | containing protein | | | | | | | |
| A0A3D9E778 | Translation initiation factor | 3.756 | 2 | 1 | 1 | 852 | 91.9 | 39.32 |
| | IF-2 | | | | | | | |
| V6A9P6 | PhoH-like protein | 2.99 | 5 | 1 | 1 | 363 | 41.2 | 69.26 |
| A0A7U3TKQ8 | HAMP domain-containing | 8.311 | 3 | 1 | 1 | 575 | 62.2 | 100 |
| | protein | | | | | | | |
| A0A3M5ELG9 | Protein translocase subunit | 30.446 | 18 | 11 | 8 | 622 | 67.9 | 32.28 |
| | SecD | | | | | | | |

Notes for Table 4:
# PSMs (the total number of identified peptide spectra matched for the protein);
FDR Confidence Combined was high for all proteins;
Exp. q-value: Combined was between 0-0.007.

Because of the rich nutrition and favorable growth conditions of the exponential growth phase, bacteria optimize their internal cellular processes to expand their population rapidly and establish biofilm communities by activating bacterial attachments. During this active growth period, bacteria likely secrete G-EVs as messengers to coordinate the cellular actions of neighbors to establish biofilm communities. The research team's analysis of the proteomic profiles of G-EVs suggests that most of the abundant proteins/enzymes found in G-EVs are those involved in cellular processes for growth, DNA synthesis and protein synthesis/processes (Table 2 and Table 3). As key cellular communication mediators, EVs are known to assist in transferring DNA, RNA, proteins, and other molecules to target cells. Because G-EVs enrich proteins that are important for cellular growth, once they are taken up by other bacterial cells, these proteins can likely enhance the bacterial functions associated with these proteins, such as cellular attachment, and promote biofilm development and cell growth.

Death and growth rates are balanced once biofilm enters the stationary phase while biofilm matures. At this stage, the bacteria within the biofilm become more resistant to antibiotics. Under normal culture conditions or in favorable growth environments, mature biofilms ultimately enter the dispersal/detachment phase, during which bacterial cells depart from the biofilm to start or participate in a new biofilm formation cycle. However, when nutrients are depleted, cells starve. This affects the biofilm in two ways. First, some bacterial cells within the biofilm die to generate nutrients by decreasing the population density (increasing the amount of nutrients per cell) and releasing nutrients through dead cells to provide nutrition for the remaining nutrient-deprived cell population. Second, to survive in such a stressful condition, the core group of biofilm bacteria rapidly adjusts its intracellular metabolic pathways, leading to alterations in intracellular components and surface proteins. Meanwhile, EVs (D-EVs) are secreted by the core bacteria to reflect these alterations. The secreted D-EVs then serve as cellular messengers to coordinate the actions of the core population of bacteria and enhance their ability to acquire vital supplies and nutrition from a resource-scarce environment.

Several unique proteins were identified on the surface of the D-EVs (Table 2), all belonging to a group of proteins that help bacterial growth by acquiring iron resources from nutrition-depleted environments. Iron is an important component of metabolic enzymes and regulatory proteins; it acts as an electron carrier and a key nutrient for bacterial life and thus plays a critical role in bacterial physiology to support growth. Therefore, it is likely that D-EVs serve as unique messengers and protein carriers to help bacterial survival under conditions of nutrition/iron deprivation by coordinating bacterial cellular functions and delivering key proteins to the recipient cells to enhance their nutrition acquisitions. However, D-EVs can only benefit recipient bacterial cell growth/survival under nutrition depletion conditions. In normal or iron-rich culture environments, D-EVs can be toxic to bacteria. For instance, as shown in FIG. 3, the presence of D-EVs can effectively inhibit the growth of parental P. aeruginosa PAO1 biofilms under normal culture conditions. Furthermore, the inhibition power of D-EVs against 96-hr. P. aeruginosa PAO1 parental biofilms can be significantly enhanced by the presence of extra Fe³⁺ (FIGS. 10A-10D).

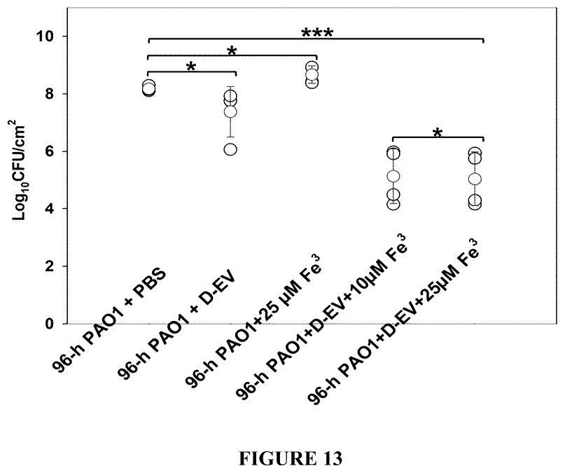

Maintaining an intracellular iron concentration of 10⁻⁶ M is important for bacterial growth; therefore, iron uptake is strictly regulated by bacterial cellular processes. Insufficient iron uptake can lead directly to cell starvation, while excessive iron uptake in a medium-rich environment can result in bacterial cell death by activating ferroptosis, an iron-dependent form of programmed cell death. A consequence of ferroptosis activated by a high intracellular concentration of iron is the triggering of the Fenton/Haber-Weiss reaction, which produces lethal concentrations of ROS, leading to cell death. This is supported by the results shown in FIG. 13, which demonstrate that P. aeruginosa PAO1 bacteria produced more overall ROS when exposed to D-EVs than when exposed to an antibiotic drug.

Together with D-EV induced increase in overall ROS, the increased intracellular H₂O₂ level (FIG. 14B) provide key evidence that exposing PAO1 biofilms to D-EVs significantly enhances the Fenton reaction within the biofilm and trigger the ferroptosis-like cell death. These observations are further supported by the finding that the abundance of the enzyme catalase in the proteomic profile of untreated PAO1 biofilms significantly increased after treatment with D-EVs (Table 3), since the expression level of catalase inside bacterial cells is directly correlated to the amount of intracellular H₂O₂.

### Example 6

### Mechanistic Investigations of Inhibition Effects of the D-EVs on Biofilm Growth.

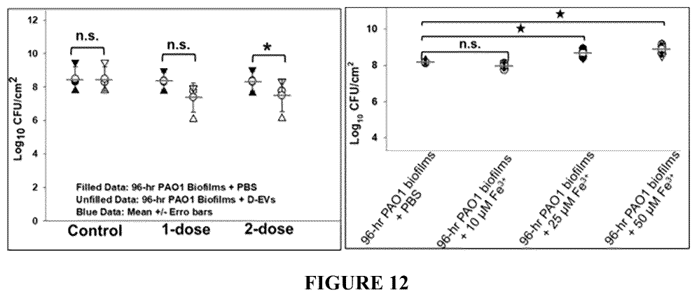

Based on the information about these unique ferric transportation/acquisition-related proteins observed on D-EV surface, the research team hypothesized that D-EVs induce excessive iron uptake by the D-EV recipient bacterial cells, resulting in the accumulation of ROS and ultimately the activation of bacterial cell death. This was tested by examining whether the efficacy of the D-EVs against aged biofilms grown for 96 hr. (FIG. 9D) could be improved in the presence of ferric ions. Compared to the PBS control, the presence of ferric ions alone marginally promoted biofilm growth, from ˜8.2−log₁₀ to 8.7−log₁₀ (FIG. 12B), while 2-dose treatments of D-EV alone only reduced the aged biofilm growth by less than ˜1−log₁₀ (FIG. 12A). However, when ferric ions were applied to the 96-h P. aeruginosa PAO1 biofilms together with D-EVs, the growth of the biofilms was significantly inhibited (FIG. 13), and the inhibition efficiency was found to be Fe³⁺ concentration dependent. As [Fe³⁺] increased from 10 μM to 50 μM, the inhibition of biofilm growth increased from a ˜2.6−log₁₀ reduction to a ˜3.4−log₁₀ reduction. These results demonstrate that both D-EVs and ferric ions are needed to improve biofilm treatment efficiency.

Using a commercial cellular ROS assay kit, it was observed that the biofilms treated with D-EVs produced much higher levels of ROS than the negative control biofilm (treated with PBS buffer) and the positive control biofilm (treated with tetracycline at 10 mg/mL) (FIG. 14). Although specific ROS species were not identified in this measurement, these results provide evidence that D-EVs promote iron uptake and induce overall ROS accumulation within the biofilm, ultimately leading to cell death.

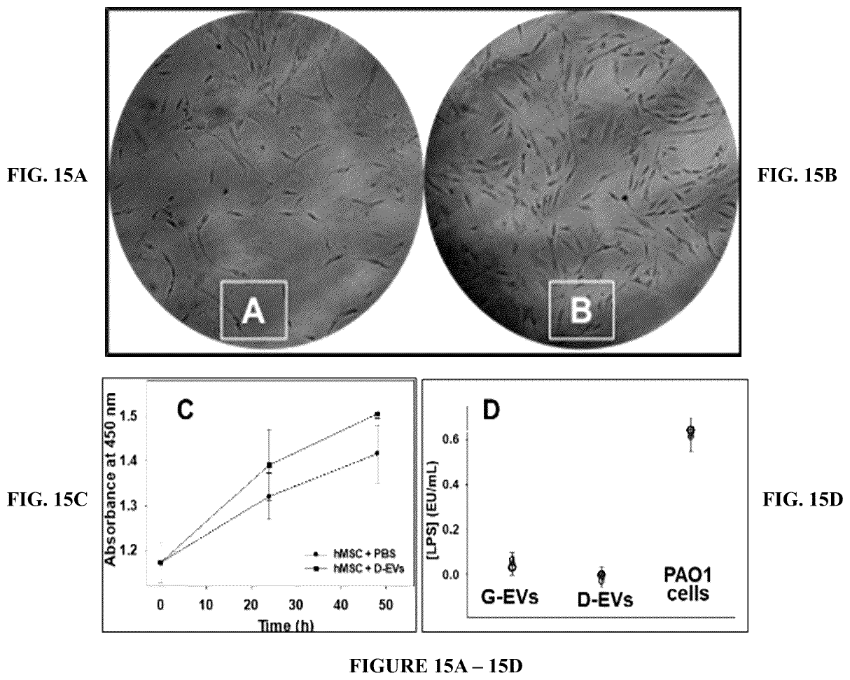

To determine the potential cytotoxicity of P. aeruginosa PAO1 D-EVs to mammalian cellular function, the research team examined the viability of human mesenchymal stem cells (hMSC) in the presence of the D-EVs (0.33 μg/μL) (see FIG. 15A-15D). The results showed no significant detrimental effects on either cell growth (FIG. 15A) or cellular morphology (FIG. 15B) of hMSC after exposure to the D-EVs for two days. These observations are consistent with earlier studies on the biosafety of using bacterial EVs in human bone and tissue therapy.

Additionally, lipopolysaccharide (LPS) tests were performed in conjunction with treatment of the hMSC cells with the purified EVs. This was pursued as LPS may potentially induce inflammatory responses in future therapeutic applications. Using a Pierce™ Chromogenic Quant Kit we found no LPS presence in D-EVs, but <0.02 EU/mL and 0.62 EU/ml of LPS in G-EV and PAO1 bacterial cells, respectively (FIG. 15D). Aligning with this observation, the proteomic data also showed that LPS biosynthesis protein was found in G-EVs and bacterial cells, but not in D-EVs; however, after treatment with D-EVs, the abundance of LPS biosynthesis protein in bacterial cells was blunted. The findings from FIGS. 15A-15D corroborate bacterial EV-based therapeutic studies supporting injection of bacterial EVs does not cause any significant side effects.

In summary, this study provides evidence of the importance of bacterial EVs in regulating biofilm growth. The regulatory roles of bacterial EVs depend on the biofilm growth stage at which the EVs are secreted. In addition, the research team discovered dual roles of D-EV in controlling biofilm growth: 1) help bacterial survival under nutrition depletion conditions and 2) treat P. aeruginosa PAO1 biofilms in the presence of Fe³⁺ ions. This discovery brings both excitement and challenge to the field. The excitement is that the discoveries, especially the Fe³⁺-enhanced inhibition power of D-EVs, provides an alternative for developing new therapies against drug-resistant bacterial infections. The challenge is that these results raise many fundamental questions. For example, how do environmental signals/conditions at each stage of bacterial biofilm development affect EV biogenesis? Do EVs secreted by other types of bacteria have the same properties and use a similar mechanism to inhibit biofilm growth? Are there other mechanisms involved?What are the roles of extracellular vesicle DNA/RNA components in the mechanisms, and how do they change when EVs are secreted by bacteria at different developmental stages?How do intracellular pathways of biomass change in response to exposure to D-EV?Answers to these questions will shed light on the regulatory roles and detailed mechanisms of bacterial EVs in biofilm growth and how the roles switch from one stage to another. Furthermore, this data set suggests that the inhibition of biofilm growth by D EVs involves ROS production. However, the nature of the ROS species involved in the inhibition is unknown. Answering this question will help pinpoint the intracellular mechanism of D-EV-induced cell death. Also, is it possible that the observed condition-dependent regulatory roles of bacterial EVs can be applied to other cellular systems?Answering this question may help address the challenges faced in the clinical application of mammalian or stem-cell-derived extracellular vesicle-based therapies for various disease treatments. Further study with other species will need to confirm effects in those species due to unpredictable nature of art.

### Example 7

### Correlating the Protein Concentration of EVs to the Particle Numbers

To correlate the protein concentration of G-EVs to their particle numbers, the protein concentration (μg/ml) of purified EVs was quantified using a Pierce™ BCA Protein Assay Kit and they were stained with Vybrant™ DiI Cell-Labeling Solution (see the Materials and Methods section). The number of fluorescent extracellular vesicle particles in each image (FIG. 5) was correlated to the protein concentration. The protein concentration is plotted against the extracellular vesicle particle number in FIG. 6, which shows a linear relationship between them.

Y(number*10³)=24450P (μg/ml)−458385 (R²=0.979)

Where Y is the extracellular vesicle particle number and P is the extracellular vesicle protein concentration (μg/ml). Based on this relationship, the average protein content of each extracellular vesicle particle is 0.00188±8×10⁴ μg/ml.

### Example 8

### Shape and Size of EVs

Ten microliters of each extracellular vesicle sample were diluted with PBS at a v/v ratio of 1:1000. Four microliters of the diluted sample were then loaded onto formvar/carbon-coated copper EM grids with a thickness of 200 nm. After drying, the grids were stained with 2% uranyl acetate (4 μl) and the excess stain was immediately removed. The grids were then allowed to dry under light for 15 minutes. Finally, the grids were examined using a transmission electron microscope (FEI Tecnai G2 20 Twin equipped with a 200 KV LaB6 electron source). TEM images of the purified G EVs and D-EVs are shown in FIG. 7. The EVs extracted during the exponential growth phase were larger (112.9±3.7 nm) than the extracellular vesicles extracted during the death/survival phase (33.2±0.9 nm).

### Example 9

### Functional Effects and Importance of Intact Extracellular Vesicle Structure of Bacterial EVs

To investigate the functional effects of D-EVs on PAO1 biofilm growth, the research team conducted a disc diffusion assay (see Materials and Methods) using both D-EVs and G-EVs to observe their functional effects on the growth behavior of P. aeruginosa PAO1 biofilm (FIG. 8A). PBS buffer and the antibiotic tetracycline were used as negative and positive controls, respectively, to assess the efficacy of D-EVs/G-EVs in affecting the growth of PAO1 biofilms. The research team used a final concentration of tetracycline of 1 μg/μl in the test to make it comparable to other treatments. G-EVs did not show an inhibition/clear zone, and D-EVs exhibited an effective and dose-dependent inhibition of PAO1 biofilm growth across a range of extracellular vesicle protein concentrations (0.11, 0.22, and 0.33 μg/μl).

Although the research team assesses that D-EV-mediated cellular communications are key to inhibiting biofilm growth, it is possible that the observed effects are caused by toxins associated with D EVs, rather than by D-EV-based cellular communications. To rule out toxin-based effects and confirm that the intact extracellular vesicle structure is critical for inhibiting bacterial biofilm growth, the team performed a diffusion susceptibility test to assess the inhibition effects of lysed D EVs. In this test, the cell walls of the extracellular vesicles were lysed by subjecting them to three 10-second water bath sonication cycles, with a 2-minute interval in ice. The results (see FIG. 8B) showed a clear zone of biofilm inhibition around the filter disc containing intact D-EV, whereas the zone around the disc containing lysed D-EV was much fainter. These results suggest that the intact extracellular vesicle structure, rather than toxin components, plays a role in regulating biofilm growth.

Two control D-EV samples were also tested on this plate to compare the inhibition effects of D EV(33° C.) and D-EV(37°) on PAO1 biofilm growth at 37° C. on TSA plate (FIG. 10B). The results suggest that the D-EVs derived at these two temperatures create similar inhibition zones on PAO1 biofilm plate at 37° C. However, close examination of the zones showed that the inhibition of D-EV(33° C.) created a clear zone, while the zone created by D-EV(37° C.) showed a few survival pathogen colonies. The reason could be that when the D-EV(37° C.) was derived from PAO1 biofilm after 96 hours of growth at 37° C., the biofilm was still in its stationary phase (FIG. 4). This would make it more likely for the D-EV(37° C.) extracted under this condition to have less inhibition efficacy than the D-EV(33° C.).

### Example 10

### Proteomic Profiles of G-EVs and D-EVs.

Analysis of the proteomic profiles of the G-EVs and D-EVs identified a total of 1099 and 987 proteins for G-EVs and D-EVs, respectively. Among these proteins, 79 surface and cytoplasmic proteins are shared by D-EVs and G-EVs (FIG. 11). The analysis also identified a total of 92 highly abundant cytoplasmic proteins that are exclusively present in G-EVs (Table 3) and a total 77 highly abundant cytoplasmic proteins that are exclusively present in D-EVs (Table 4).

### Proteomic Analysis of Proteins Unique to Death-Phase EVs Versus Growth-Phase EVs.

In addition, a proteomic analysis of the proteins expressed in death phase EVs versus growth phase EVs was conducted. Approximately 90 proteins were found to be exclusive to death EVs harvested from P. aeruginosa biofilms compared to growth EVs. These proteins are shown in Table 5.

TABLE 5
Select proteins unique to death phase EVs compared
to growth phase EVs, with their function and
relevance to antimicrobial mechanisms.

| Protein | | | Presence | Presence |
|---|---|---|---|---|
| (Identifier) | Function | Category | in G-EVs | in D-EVs |
| HasA (Heme | Scavenges | Iron | Not | High in |
| acquisition | heme iron | uptake | detected | D-EVs |
| protein) | for uptake | | in G-EVs | (iron- |
| | | | | starvation |
| | | | | response) |
| Ferric- | Uptakes | Iron | Not | High in |
| siderophore | ferric- | uptake | detected | D-EVs |
| receptor (FptA) | pyochelin | | in G-EVs | (iron- |
| | complexes | | | starvation |
| | | | | response) |
| TonB- | Uptakes | Iron | Not | High in |
| dependent | ferric | uptake | detected | D-EVs (iron- |
| receptor (PfeA) | enterobactin | | in G-EVs | starvation |
| | (siderophore) | | | response) |
| Trans- | Shifts | Redox | Not | High in |
| hydrogenase | NADPH/ | modulation | detected | D-EVs |
| (PntAB) | NADH | | in G-EVs | (induces |
| | balance | | | oxidative |
| | (impacts | | | stress) |
| | ROS) | | | |
| AHL acylase | Degrades | QS | Not | High in |
| | AHL | interference | detected | D-EVs |
| | quorum | | in G-EVs | (blocks |
| | signals | | | competitor |
| | | | | signaling) |
| Flagellin (FliC) | Flagellar | Structural | Not | Present in |
| | filament | (flagella) | detected | D-EVs (cell |
| | protein | | in G-EVs | lysis |
| | (motility) | | | byproduct) |
| OprD-family | Outer | Membrane | Not | Present in |
| porin | membrane | transport | detected | D-EVs |
| | nutrient | | in G-EVs | (starvation- |
| | channel | | | induced) |
| TetR-family | Transcriptional | Regulatory | Not | Present in |
| regulator | repressor | protein | detected | D-EVs |
| | (various genes) | | in G-EVs | (stress |
| | | | | response) |

### Impact of Iron Supplementation on Death EV Proteomic Profile.

A comparison of the proteomic profiles of death EVs and death EVs harvested when P. aeruginosa was grown in the presence of iron was additionally evaluated. It was found that the addition of exogenous iron during D-EV induction causes distinct shifts in the proteins produced within the vesicles. Table 6 demonstrates some representative differences.

TABLE 6
Proteomic differences in death phase EVs and
death phase EVs supplemented with iron.

| | | Abun- | Abun- | |
|---|---|---|---|---|
| | | dance | dance | Change |
| | | in D-EVs | in D-EVs | with Fe |
| Protein | Role in D-EVs | (No Fe) | (+Fe) | Addition |
| HasA | Iron | High | Low/Not | Decreased - |
| (heme | scavenging | (strongly | detected | repressed by |
| acquisition | cargo | present) | | excess iron |
| protein) | | | | |
| Ferric- | Uptake of | High | Low/Not | Decreased - |
| siderophore | ferric- | (strongly | detected | less needed |
| receptor | siderophore | present) | | with iron |
| (FptA) | complexes | | | available |
| Fe-pyochelin | Siderophore | High | Low | Decreased - |
| receptor | uptake | | | iron |
| | (pyochelin) | | | supple- |
| | | | | mentation |
| | | | | suppresses |
| | | | | expression |
| Pyridine | Induces ROS | High | High | No change/ |
| nucleotide | via redox | | | Increased - |
| trans- | imbalance | | | remains |
| hydrogenase | | | | abundant |
| (Pnt) | | | | to promote |
| | | | | ROS |
| Quinone | Potential | Moderate | Moderate/ | No change |
| oxidore- | H<sub>2</ | | High | or ↑ - |
| ductase | sub>O<sub>2</ | | | stress |
| (ROS | sub> producer | | | enzyme |
| generator) | | | | persists with |
| | | | | iron |
| AHL acylase | Degrades | High | High | No change - |
| (quorum | quorum-sensing | | | still |
| quencher) | molecules | | | packaged |
| | | | | for QS |
| | | | | disruption |
| Catalase/ | Detoxifies | Low | Low/ | ↑ Slight - |
| peroxidase | peroxide | (minimal in | Moderate | may increase |
| (antioxidant) | (if present) | D-EVs) | | to manage |
| | | | | iron-induced |
| | | | | ROS |

### Example 11

### Mechanistic Investigations of Inhibition Effects of D-EV on Biofilm Growth.

To test if the suppression of PAO1 biofilm growth is caused by D-EV-induced excessive iron uptake by D-EV recipient cells leading to the activation of bacterial cell death, the research team examined whether D-EV-induced excessive iron uptake could improve the poor efficacy of the D EVs against 96-h biofilms (FIG. 9D). Briefly, the team first treated 96-h PAO1 biofilms with 2 doses of D-EV at a protein concentration of 0.33 μg/μL with a 12-hour interval, which only induced an inhibition of less than 1-log 10 (FIG. 12A). The team then repeated the same inhibition experiments applying 10 μM, 25 μM, or 50 μM of Fe³⁺ directly to the 96-h PAO1 biofilms to see how iron alone affects the growth of 96-h PAO1 biofilms. Compared to the PBS control, the presence of ferric ions alone marginally promoted biofilm growth with a dose-dependent effect (FIG. 12B).

### Example 12

Cell Viability of D-EVs Derived from PAO1 Biofilms.

The research team investigated the toxicity of PAO1 D-EVs (0.33 μg/μl) to mammalian cell viability. Human mesenchymal stem cells (hMSC) obtained from ATCC (Manassas, VA) were cultured in alpha-MEM medium with 5% FBS, 1% L-glutamine, and 1% Pen-Strep in an incubator with 5% C02 at 37° C. Cells were then used to seed 6-well plates with an inoculation rate of 10\hat{ }4 and cultured to 60-70% confluency before being exposed to D-EV (0.33 μg/μl) or PBS (as a control) in separate cultures of hMSC. After 24 hours and 48 hours of exposure, the growth and cellular morphologies of the hMSC were examined. The results, depicted in FIGS. 15A-15B, showed no significant detrimental effects on either cell growth or the cellular morphology of hMSC after two days of exposure to D-EVs. These results demonstrate that the presence of D-EVs does not affect cellular functions and thus provide a foundation for exploring the potential use of D-EVs in antibiotic applications.

### Example 13

Inhibition of C. auris Growth by Bacterial D-EVs.

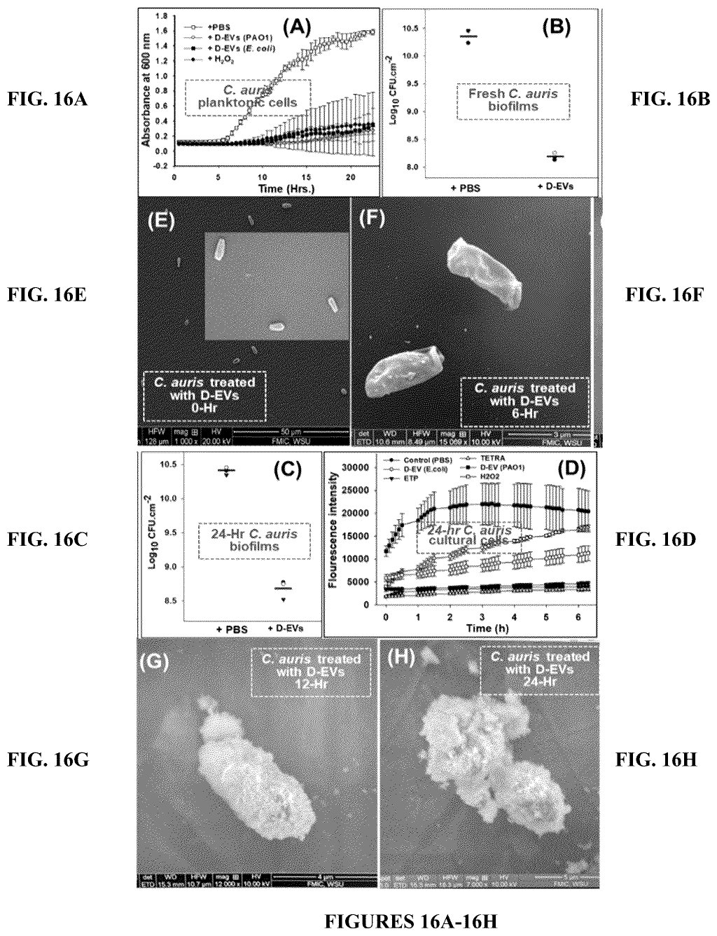

To demonstrate the inhibition by bacterial D-EVs of C. auris growth, the cell growth and viability of C. auris planktonic cell culture was examined (FIG. 16A), freshly formed biofilms (FIG. 16B) and aged biofilms (FIG. 16C) after one-dose treatment with bacterial D-EVs. As expected, the results show that bacterial D-EVs can inhibit fungal biofilm growth compared to controls, even with one-dose treatment. We also investigated how the ROS levels of C. auris samples respond to treatment with bacterial D-EVs. The results, shown in FIG. 16D, suggest that the observed inhibition of fungal growth is due to the bacterial D-EVs triggering accumulation of intracellular ROS within C. auris fungal cells and biofilms. Examination with scanning electron microscopy (SEM) (FIGS. 16E-16H) showed that D-EV treatment had led to the destruction of fungal cellular membranes and cell death.

The experimental findings presented in the following example provide basis for the mechanistic roles of bacterial D-EVs in interrupting fungal cellular signaling processes and the potential application in combatting antibiotic resistant fungal infections. This support of bacterial D-EVs on fungal biofilms is supported by the use of D-EVs derived from PAO1 biofilms, bacterial D-EVs derived from E. coli biofilms were both effective against C. auris growth (FIGS. 16A, 16B).

### Mechanistic Investigation of the Roles of Bacterial D-EVs in Inhibiting C. Auris Biofilm Growth.

To investigate if bacterial D-EVs carry common signaling molecules that shift the cellular redox balance of C. auris to a more oxidative state and alter the intracellular signaling pathways of the fungal cells, thus triggering the accumulation of ROS within the C. auris biofilms. D-EVs extracted from biofilms formed by PAO1 and E. coli bacteria at their survival/death stage under nutrition-limited conditions following protocols established in the preceding examples. Qualitative tests demonstrated that both planktonic C. auris cells and C. auris biofilms can be inhibited by bacterial D-EVs (FIGS. 16A-16C).

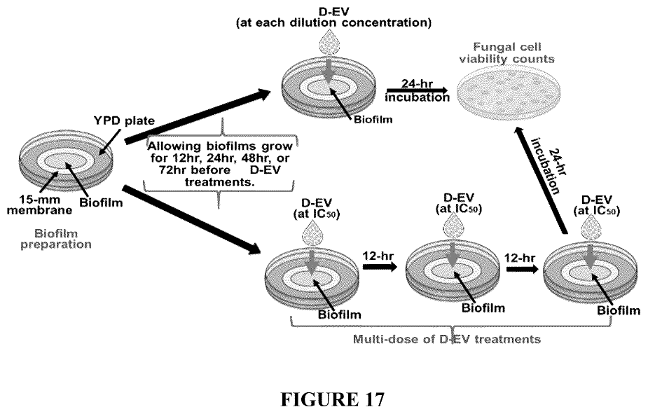

Candida auris (B11220, ATCC) frozen at −80° C. was activated in yeast peptone dextrose (YPD) broth overnight at 37° C. Cells were be maintained at 4° C. on YPD-agar plates. The in vitro C. auris biofilms were be prepared by first activating the cultures at 4° C. in YPD broth at 37° C., then transferring a volume of 2.5 μL (OD₆₀₀˜0.5) onto sterile 13-mm polycarbonate membrane (WHA10417401, Sigma-Aldrich) on YPD-agar plates (FIG. 17). The plates were then incubated at 37° C. for 0 h, 24 h, 48 h, and 72 h, respectively, for the following experiments.

Treating In Vitro C. auris Biofilms Using Bacterial D-EVs

If bacterial D-EVs can inhibit the growth of biofilms formed by C. auris at various developmental stages will be investigated qualitatively and quantitatively. The quantitative efficacy profile of D-EVs from both PAO1 and E. coli biofilms using minimal inhibitory concentration (ICₘᵢₙ) and half maximal inhibitory concentration (IC₅₀) against C. auris biofilms at various developmental stages.

Determining IC₅₀ and ICₘᵢₙ of Bacterial D-EVs against C. auris biofilms at each Stage

Briefly, based on the well-developed protocol described in the preceeding examples, serial dilutions of the D-EVs will be performed, and biofilm plates at each stage will be treated with the D-EVs at each dilution concentration and incubated at 37° C. for 24 hr. before viable cell counts. To determine viable cell concentration (CFU/cm²), the treated biofilms will be suspended in 0.9% NaCl to prepare serial dilutions for CFU counting^{76,77}. FIG. 17 shows a general protocol for this experiment. The concentration of D-EVs vs. CFU plot will provide IC₅₀ and ICₘᵢₙ for both PAO1 and E. coli D-EVs against C. auris at each growth stage. The results will provide guidelines for concentration selection for the rest of the experiments to assess the efficacy of the D-EVs.

### Assessing Efficacy of the D-EVs Against C. Auris Biofilm at the Different Mature Stages.

As illustrated in FIG. 17, 4 samples of C. auris biofilms, after each of 0, 24, 48, and 72 hr. of growth, will be treated with 3 doses (which can be further optimized) of D-EVs from either E. coli or PAO1 at the IC₅₀ concentration for that growth stage; this will require at least 24 experiments with at least 4 biological replicates. Through these dose-dependent tests, we will be able to establish the efficacy profile of each D-EV at its IC₅₀ for C. auris biofilm (like the results shown in FIG. 9C) at the various developmental stages.

In Vivo Structural Analysis of Biofilms Responding to Treatment with Bacterial D-EVs.

Microbial biofilms are dynamic systems, characterized by variations in size, matrix composition and internal architecture throughout their growth. Microbial cells within biofilms, including those of C. auris, can rapidly adapt their intracellular processes, resulting in changes to intracellular components and biofilm structure in response to environmental stimulations, which can affect the penetration of drugs into biofilms. The objective of this experiment is to investigate the real-time changes in the surface tomography and structural dynamics of living C. auris biofilm matrices under treatment with either PAO1 or E. coli D-EVs. Fungal biofilms will be grown in single-pass biofilm reactors at a controlled temperature and continuously exposed to the selected bacterial D-EVs at its IC₅₀. Imaging of the fungal biofilms will be performed using a Nikon D-Eclipse CI CLSM at regular intervals (every 4 hours) over a 48-h period. Changes in biofilm structure will be quantified. The observed biofilm structural changes in response to D-EVs will provide additional information on the mechanisms of action of bacterial D-EVs against fungal biofilms.

### Examination of the Accumulation of Intracellular ROS of Fungal Cells Induced by Bacterial D-EV Treatments.

In FIG. 16D, it was observed that a significant increases in overall ROS release by fungal cells followed exposure to bacterial D-EVs; however, no specific ROS was identified. The objective of these experiments is to identify specific ROS species involved in bacterial D-EV induced C. auris cell death. Our treatments of C. auris cells with bacterial D-EVs could stimulate production of intracellular radical ROS that ultimately leads to fungal death.

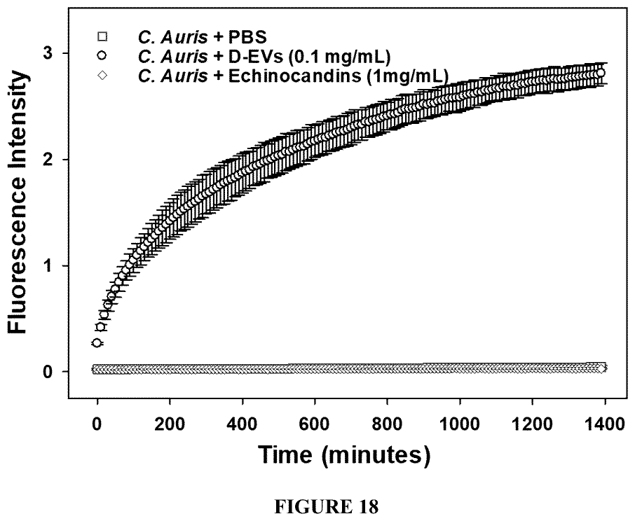

In these experiments commercial cell-based assay kits (ab219931 and ab139476, Abcam) and microelectrodes will be used to monitor changes in intracellular concentrations of radical ·OH and O²⁻ generated by C. auris biofilms in the presence of bacterial D-EVs at IC₅₀ concentrations, Commercial assays provide average concentrations, while microelectrodes measuring H₂O₂ provide local concentrations inside the biofilm. It is known that the intracellular level of H₂O₂ plays a critical role in regulating the Fenton reaction and ferroptosis. In another test, it was observed that a significant increase in intracellular H₂O₂(FIG. 18) occurred when C. auris cells were treated with PAO1 D-EVs (0.10 μg/μL), indicating a link between bacterial D-EV treatment and enhanced ferroptosis. In future experiments, a H₂O₂ detection kit (ab138874, Abcam) and our microelectrodes to quantify further the changes in the intracellular level of H₂O₂ after C. auris is treated with PAO1 or E. coli D-EVs at their IC₅₀. will be utilized. The microelectrodes enable mapping H₂O₂ levels at different depths within a biofilm in response to D-EV treatment

For the H₂O₂ detection assays, the changes in the level of each ROS species will be monitored using the CYTATION 5 (BioTek/Agilent) imaging plate reader. By comparing the results with those for negative (PBS treatment) and positive (tetracycline at 10 mg/mL or ertapenem at 1.0 μg/μL) controls, the impact of D-EVs on C. auris intracellular ROS concentrations will be assessed and the correlation of the increase in each individual ROS to D-EV induced fungal cell death kinetics and specific intracellular pathway will be fulfilled.

To test further whether the observed increase in radical ROS is directly linked to D-EV induced fungal cellular death, C. auris biofilms will be treated with 50 mM thiourea, a potent scavenger of hydroxyl radicals in eukaryotic and prokaryotic cells, for 30 min prior to the addition of a bacterial D-EV at its IC₅₀. If fungal death is mitigated by the presence of thiourea, this will indicate D-EV induced ROS production plays a critical role in fungal death. These results will provide critical insights into the mechanism of D-EV induced inhibition of fungal biofilm growth, shed light on the potential mechanism of ROS overproduction, and provide valuable data on the efficacy of bacterial D-EV inhibition of fungal biofilms.

Mechanistic Study of Inhibition of C. auris Biofilms by Bacterial D-EVs.

Previous examples herein of bacterial biofilms showed the inhibitory power of bacterial D-EVs against PAO1 biofilms can be significantly enhanced by the presence of extra Fe³⁺ (FIGS. 14A, 14B, 13) though activation of ferroptosis. Ferroptosis is a regulated pathway of cell death characterized by iron accumulation and lipid peroxidation, which lead to the iron-dependent overproduction of ROS to lethal levels. However, as suggested by the results listed in Table 2, other mechanisms may also be involved in ROS overproduction. Treatment of C. auris cells with bacterial D-EVs could alter the fungal intracellular redox balances and activate ferroptosis-based fungal cell death. Therefore, the potential role of ferroptosis and GSH/GSSG ratio as an intracellular redox potential in the observed inhibition of C. auris biofilm growth by bacterial D-EVs will be examined.

To examine the synergistic inhibitory effects of ferric ions and bacterial D-EVs on the growth of fungal biofilms, C. auris biofilms in the initial and exponential growth phases (2 hr. and 8 hr. of growth respectively) and at the mature stage (24 hr. of growth) will be treated with both PAO1 and E. coli D-EVs at their IC₅₀ in the presence of 10 μM, 20 μM, and 30 μM ferric ion concentrations following the experimental protocol described in FIG. 17. If ferroptosis is indeed a primary mechanism, it is anticipated that there will be an iron-enhanced efficacy of bacterial D-EVs against C. auris biofilm growth. The results will be further verified by applying lipophilic antioxidants.

Lipophilic antioxidants such as liproxstatin-1 and ferrostatin-1 have been shown to block intracellular pathways associated with ferroptosis. Ferrostatin-1 has been used to suppress ferroptosis in Mycobacterium tuberculosis infection. In this experiment, C. auris biofilms will be grown under normal YDP culture conditions. Once the biofilms reach the mature stage (24 h), they will be treated with bacterial D-EVs in the presence and absence of 10 μM ferrostatin-1. Quantitative analysis of viable cell numbers will be performed at 12 hr., 24 hr., and 36 hr. after the treatments. If ferrostatin-1 mitigates the D-EV-induced death of C. auris biofilms, this will support a mechanism whereby ferroptosis is the primary mechanism leading to D-EV induced fungal death because the presence of ferrostatin-1 blocks D-EV induced ferroptosis.

The GSH/GSSG ratio and the NADH/NAD⁺ cycle play crucial roles in maintaining microbial cellular redox balance to regulate ROS generation. Proteomic data shown in Example 5 show that D-EVs exclusively carry several proteins, including extracellular heme-binding proteins and TonB-dependent receptors, as listed in Table 2, that play roles in regulating bacterial intracellular redox balances, and the expression levels of these enzymes in biofilms are significantly increased after exposure to D-EVs (Table 1). These enzymes primarily regulate microbial intracellular redox balance by converting GSH to GSSG or NADH/NADPH to NAD⁺/NADP⁺, but under adverse conditions (e.g., D-EV treatment) upregulation of these enzymes affects the intracellular redox potentials and shifts the balances to more oxidative stress states by reducing GSH, NADH or NADPH levels, promoting ROS production. Monitoring changes in the GSH/GSSG and NADH/NAD⁺ ratios of C. auris biofilm at different growth stages in response to D-EV treatments will be performed. The GSH/GSSG and NADH/NAD⁺ ratios will be determined using a Glutathione GSH/GSSG Assay Kit (MAK440-1KT, Sigma-Aldrich) and an NAD/NADH Assay Kit (ab65348, Abcam), respectively, following the manufacturers' instructions. The results will be correlated to the viable cell concentration (CFU/cm²) under each condition.

Candida cells respond to oxidative stress by inducing genes encoding catalase (CAT) to detoxify ROS and restore redox homeostasis. Cat1 plays a major role in protecting Candida cells from peroxide stress by elevating the intracellular catalase level, enhancing the conversion of H₂O₂ to water. If bacterial D-EV induced ferroptosis is the primary mechanism leading to the observed inhibition of fungal growth (FIG. 16A), it will be expected that the intracellular catalase level will be significantly increased after C. auris biofilms are exposed to bacterial D-EVs. In this experiment, an EnzyChrom™ Catalase Assay Kit (BioAssay System) will be used to monitor changes in the basal catalase level of C. auris cells after exposure to D-EVs derived from PAO1 and E. coli. To further explore the role of ferroptosis in bacterial D-EV induced fungal death further, a catalase-deficient C. albicans Δcat1 cell (in which the cat1 gene is nulled) as the model will be used to examine the role of each of the bacterial D-EVs in disrupting the intracellular redox homeostasis of C. auris cells. Both the intracellular catalase level and the viable cells (CFU/cm²) of catalase-deficient albicans Δcat1 and isogenic wild-type albicans will be measured before and after exposure to each bacterial D-EV type. If catalase-deficient cells are more susceptible to D-EV treatment than wild-type fungal cells, this will further support bacterial D-EV induced ferroptosis in activating C. auris cell death.

Proteomic Analysis of C. auris Biofilms after D-EV Treatment.

Because of the dynamic nature of microbial biofilms during their growth, microbial cells within biofilms can rapidly adapt their intracellular processes in response to environmental cues. Based on results listed in Table 2, which shows several unique bacterial EV proteins that potentially enhance ferroptosis and cellular ROS production of the D-EV recipient cells, in response to taking up bacterial D-EVs, C. auris biofilms will alter their cellular processes toward their programmed cell death pathways. To gain a comprehensive understanding of how the intracellular processes of fungal biofilms respond to bacterial D-EV treatments, proteomic analysis will be performed on both PAO1 and E. coli D-EVs and biomasses of C. auris biofilms treated with the selected bacterial D-EVs at their IC₅₀ under a variety of conditions.

C. auris biofilms at each developmental stage (e.g., 0 h, 24 h, and 48 hr. of growth) will be treated with the selected bacterial D-EVs. Followed by collection of the fungal biomass samples at specified time points following D-EV treatment (as outlined in Table 2) for proteomic analysis. In the analysis of P. aeruginosa biomass treated with PAO1 D-EVs, it was observed that an abundance of certain intracellular bacterial proteins significantly increased over time, while the same proteins were observed to start to diminish in control samples. Similar D-EV induced changes in the proteomic profiles of fungal biofilms is expected. Of particular interest are the changes in Candida genes encoding ferric reductases (e.g. CFL5, FRP1), high-affinity iron permeases (e.g. FTR1) and proteins involved in iron assimilation (e.g. FET3), which are known to play important roles in regulating iron homeostasis and fungal survival. Changes in the proteomic profile of Candida genes encoding CAT1, superoxide dismutases (SOD), glutathione peroxidases (GPX) and components of the glutathione/glutaredoxin (GSH1, TTR1) and thioredoxin (TSA1, TRX1, TRR1) systems, which play critical roles in responding to oxidative stress by detoxifying ROS, repairing oxidative damage, synthesizing antioxidants and restoring redox homeostasis of fungal cells are also expected. A proteomic comparison of bacterial D-EVs treatment of C. auris cells at various growth stages, along with an analysis of the proteomic profile changes in C. auris after D-EV treatments, will offer insights into the inhibitory mechanisms of bacterial D-EVs against C. auris biofilms.

TABLE 7
Biofilm biomass collection scheme after D-EV treatment.

| | | Collection time of biomass of | | |
|---|---|---|---|---|
| C. auris | Treated | biofilms after D-EV treatments | | |
| biofilm | with D-EVs | 0 h | 12 h | 24 h |
| Fresh biofilms (8 h) | No | X | X | X |
| | Yes | X | X | X |
| 24 hr. mature | No | X | X | X |
| biofilms | Yes | X | X | X |
| 48 hr. mature | No | X | X | X |
| biofilms | Yes | X | X | X |

*D-EVs derived from PAO1 biofilms and E. coli biofilms.

### Investigation of the Potential Development of Resistance of Fungal Biofilms to Bacterial D-EV Treatments.

Microbial antibiotic resistance can be either developed intrinsically or acquired extrinsically through interaction with the environment. Therefore, if C. auris biofilms will develop resistance to bacterial D-EV treatments will be investigated. Since Candida cells tend to grow together with Gram-negative bacteria such as P. aeruginosa and E. coli in biofilms, it is expected that C. auris biofilms will be less likely to develop resistance to interacting with the D-EVs derived from bacterial biofilms. To test this, a volume of 2.5 μL (OD₆₀₀=0.5) of C. auris will be used to seed sterile 13-mm polycarbonate membrane (WHA10417401, Sigma-Aldrich) on YPD agar plates and incubated at 37° C. Once the fungal biofilms grow into the mature phase (after 24 h), three doses of each bacterial D-EV at its IC₅₀ will be applied (one dose every 24 h). Counting colonies will be prepared from the plates of treated fungal samples every 12 hr. for biomass analysis to calculate CFU/cm² reduction after D-EV treatment. A reduction of 3−log₁₀ CFU/cm² (FIGS. 16B & 16C) will be expected for the first round of treatment. Surviving cells will be streaked on YPD and incubated overnight at 37° C. Following the same protocol, the surviving cells from the second round of D-EV treatment will be subjected to the next round of D-EV treatment and analysis. This procedure will be repeated until the 10^{th} generation to determine potential resistance following protocols from previous work. By comparing the results of biomass analysis from all rounds of D-EV treatment, we will be able to assess the resistance of the fungal biofilms to the bacterial D-EV treatments. However, no statistically significant resistance is expected.

Comprehensive examination of the inhibitory effects of bacterial D-EVs on fungal biofilm growth described in this prophetic example will allow for quantitative assessment of the potential of bacterial D-EVs against C. auris biofilms. Validation of the proposal of ferroptosis for the redox ratios/balances and its relationship in C. auris biofilm growth will allow for a comprehensive understanding of the unique intracellular pathways governing C. auris biofilm growth and death. The identification of key signaling pathways and proteins/enzymes related to bacterial D-EV induced fungal death will give insights into the interspecies communication pathways established by the bacterial D-EVs on the C. auris culture.

Examination of the inhibition of two different bacterial D-EVs on the growth of C. auris biofilms will also offer insights into potential common mechanisms for the effects of D-EVs.

### Example 14

### In Vitro, Ex Vivo and In Vivo Assessments of Bacterial D-EVs for Cellular Viability and Therapeutic Potential.

Ex vivo investigation of inhibition efficacy and toxicity of bacterial D-EVs using a porcine explant biofilm model.

Because of its convenience, vascular nature, and structural similarity to human dermis, porcine explants have been extensively utilized as an ex vivo model for studying bacteria and/or fungal tissue interactions, evaluating antimicrobial agents, assessing cytotoxicity, and investigating wound biofilms and treatment efficacy. A porcine explant biofilm model will be used to assess the effectiveness of each of the bacterial D-EVs for preventing biofilm formation and treating preexisting biofilm. With the expectation that the bacterial D-EVs will have no effect on mammalian cellular functions.

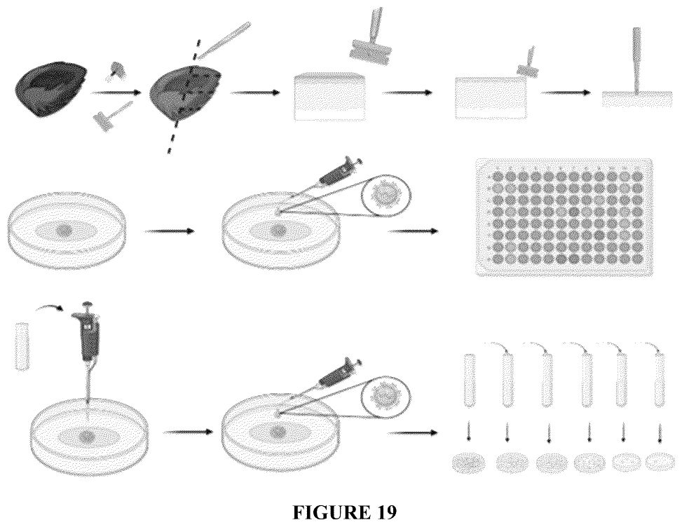

As illustrated in FIG. 19, healthy pig (Sus scrofa domesticus) ears from local butchers will be used after being shaved and cleaned. Where the porcine explants will be prepared by carefully removing a 500-μm-thick layer of skin from an ear strip and another 500-μm-thick layer primarily consisting of dermis will be sliced from each strip. Circular discs with a diameter of 12 mm will be punched out from the dermal layer. The prepared discs will be placed in insert wells with a membrane immersed in 2 mL of Dulbecco's modified Eagle medium (DMEM) and be incubated at 37° C. C. auris culture with or without the bacterial D-EV at IC₅₀ concentration will be introduced onto the explants. The aim of the explant porcine model is to mimic in vivo conditions and evaluate the interactions between the bacterial D-EVs and C. auris biofilms in a controlled ex vivo setting. G-EVs from PAO1 will be used as a negative control. It is expected that the G-EVs will promote biofilm infection whereas the D-EVs will prevent infection.

To evaluate the inhibitory efficacy of the bacterial D-EVs against both fresh and mature dermal explant biofilms, the infected explant models will be established by inoculation with 10 μl of 0.5 McFarland of C. auris. These models will be divided into two subgroups: a control group without D-EV treatment and a treatment group receiving the D-EVs. For the treatment group, the D-EVs will be applied either immediately after fungal infection or after the fungal biofilm has been allowed to mature for 24 h. The explants will be incubated with the D-EVs for a specified duration. Fungal biofilms on the infected explants will be quantified. By comparing the results obtained from the D-EV treatment group to those of the non-D-EV treatment group, the inhibition efficiencies of the bacterial D-EVs on fungal biofilm growth in the porcine explant models will be able to be determined.

To assess the cell viability of porcine explants treated with bacterial D-EVs, the explants will be placed onto 13-mm membranes with a 0.2-μm pore size on TSA plates. These explants will not be subjected to fungal infections. After 24-48 hr. of D-EV treatment, they will be transferred to a 96-well plate containing 180 μl of DMEM and 20 μl of PrestoBlue cell viability assay (ThermoFisher, cat. no. A13261). Established protocols will be used to evaluate cell viability.

### Inhibition by D-EVs in a Murine Wound Infection Model

It remains important to validate the use of bacterial D-EVs in preventing fungal biofilm formation in wound beds, treatment of preexisting biofilms, and promotion of wound healing in a fungal disease model. Results presented in the preceding examples provide evidence for D-EV use in a bacterial murine wound bed; however, the use of D-EVs in a fungal biofilm wound remain to be corroborated. Additionally, the use of D-EVs in an immunocompromised mouse model will further address the utility of D-EVs as a treatment.

### Preparation and Test of Murine Wound Biofilm Model.

After shaving and disinfecting the skin on the dorsum of a mouse, an immunocompromised BKS.Cg-Dock7^{m}+/+Lepr^{db}/J mouse acquired from the Jackson Laboratory, a circular, full-thickness skin wound with a diameter of 5 mm will be created using a sterile skin puncture biopsy tool (Acuderm Inc., Fort Lauderdale). The wounds will be infected with either 15 μL of 0.9% sterile saline (as the control animal group) or with 15 μL of C. auris at a concentration of approximately 108 CFU/ml and will be the experimental group. Each of these groups will be further divided into two Subgroups: I and II.

Subgroup I (prevention protocol): After creating the infected wounds, the wound bed will be promptly treated by loading 10 μL of 1) PBS (treatment control) or 2) either a PAO1 or an E. coli D-EV at its IC₅₀ with/without ferric ions at 20 μM or 30 μM and ertapenem at 1.0 μg/μL, then the wounds will be covered with transparent waterproof bandages (Eseige). These experiments are designed to assess the efficacy of D-EVs in preventing or inhibiting biofilm formation on infected wound beds.

Subgroup II (treatment protocol): The infected wound beds will not be treated for two days. During the initial 2-day period without treatment, fungal biofilms will form and mature on the infected wound beds. On day 3 (after 48 hr. of biofilm growth), the biofilm-infected wound beds will be treated by loading 10 μL of 1) PBS (as a control), 2) either a PAO1 or an E. coli D-EV at its IC₅₀ concentration with/without ferric ions, or 3) ertapenem at 1.0 μg/μL. These experiments are designed to examine the effectiveness of each bacterial D-EV in eliminating existing biofilms that have already formed on wound beds.

In both subgroups, the treated animals will be monitored for a period of five days, and daily assessments of the wound treatments will be performed. The diameters of the wounds will be measured and recorded daily. Any reduction in the measured wound area will indicate progress in wound healing.

### Assessment of the Wound Treatment.

The wound biofilm will be quantified by carefully removing the wound tissue removed using a 10-mm biopsy punch tool, weighed, and then homogenized using a tissue homogenizer. The homogenized tissue will then be sonicated in 1 ml of sterile 0.9% saline. To quantify the C. auris load, 100 μl of this suspension will be serially diluted in 0.9% saline. CFUs will be determined by spread-plating 100 μl of each dilution and incubating it at 37° C. for 24 h. The quantification will be reported as CFU/g.

Via the in vivo wound biofilm will also be assessed via SEM, where the 10-mm biopsy tissue containing the wound bed will be fixed and dried in a vacuum sputter coater. Subsequently, it will be sputter-coated with a layer of gold/palladium (60%/40%) and analyzed using a cold-field emission scanning electron microscope. This will provide high-resolution images of the wound biofilm structure and composition.

The histopathology of the in vivo wound biofilm samples will be assessed. Here, the 10-mm skin tissue containing the wound bed will be fixed in 10% formalin to preserve the tissue structure. The tissue will be processed, embedded in paraffin, and sectioned into thin slices. These will be stained using histological stains such as hematoxylin and eosin (H&E) to assess the changes in the samples. The histopathology images will be reviewed by trained and certified veterinary pathologists.

To evaluate the potential toxicity and inflammation of the D-EVs, the animal blood samples will be collected and analyzed using a Piccolo® Xpress™ Chemistry Analyzer to measure the levels of blood inflammatory and pro-inflammatory biomarkers such as IFN-7, IL-10, IL-12p70, IL-2, IL-4, IL-5, IL-6, TNF-α, and KC/GRO. These biomarkers play multiple roles in wound healing, including promoting epithelialization through the clearance of apoptotic cells. Assessing these biomarkers will provide insight into the potential of our D-EVs for promoting the healing of chronic wounds at the cellular and metabolic levels.

Importantly, the levels of H₂O₂ will be measured using microelectrodes. Daily, the levels of H₂O₂ will be mapped at different layers of the biofilms, wound bed, and inside the tissue during treatment under all evaluated conditions. Together with the results from other assessments of the wound healing, the obtained H₂O₂ concentration depth profiles will provide critical in vivo information on the healing process of the C. auris infected wound under bacterial D-EV treatments. It is expected that these measurements will provide further in vivo evidence to support the inhibition mechanism.

The results from these experiments will provide valuable insights into the in vivo efficacy and safety for treating fungal biofilm infections of each bacterial D-EV at its IC₅₀ concentration. By evaluating the ability of D-EVs to inhibit fungal biofilm formation and treat mature biofilms, a comprehensive understanding of the therapeutic potential of bacterial D-EVs for treating fungal biofilm infections on immunocompromised animal wounds will be obtained.

### Example 15

Mechanistic Study of Inhibition of Bacterial Conditional Extracellular Vesicles Against Pseudomonas aeruginosa Biofilms.

Bacterial extracellular vesicles (EVs) are released during biofilm dispersal stage. These EVs, extracted at the death phase of bacterial growth, have been shown in the preceding examples to prevent biofilm formation and inhibit the growth of mature biofilms. In this example, the proteomics profiles of EVs extracted during the death phase (D-EVs) were compared to biofilms treated with D-EVs with and without iron. Additionally, the mechanisms through which the D-EVs act on the biofilm including production of ROS, production of superoxide through NADPH and Ca²⁺ imbalance was investigated.

Iron is a macronutrient supplement for bacterial growth, but high concentrations of iron (>100 μM) is generally considered toxic and results in cell damage. Therefore, the inhibition efficacy of D-EVs when supplemented with low concentration of iron (50 μM) on the growth of P. aeruginosa PAO1 biofilms was measured.

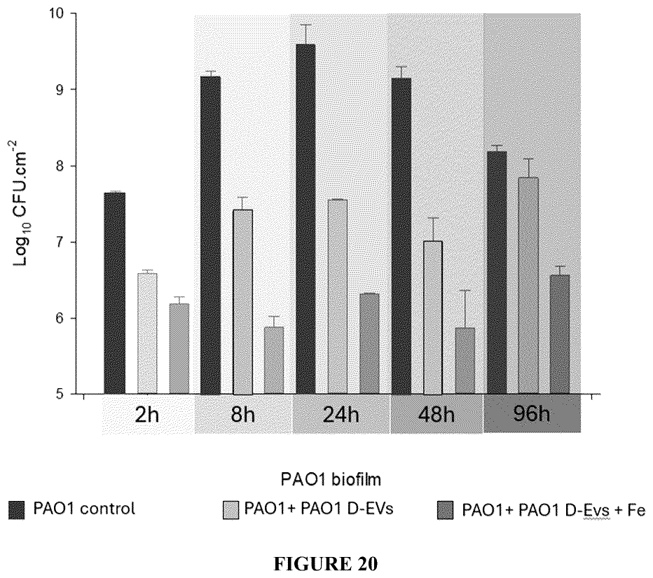

The viability of treated P. aeruginosa PAO1 biofilms was investigated and compared to control. P. aeruginosa PAO1 biofilms at 2 hr., 8 hr., 24 hr., 48 hr., and 96 hr. were individually treated with a one-dose of P. aeruginosa PAO1 D-EVs±iron (50 μM) and the biofilm inhibition was quantified in the term of log₁₀ CFU·cm⁻². As shown in FIG. 20, which displays the inhibition log₁₀ CFU·cm⁻² of P. aeruginosa PAO1 biofilms. FIG. 20 indicates that the growth of PAO1 biofilms increased when supplemented with iron and decreased when treated with D-EVs. Inhibition was further increased when iron was supplemented.

### Free Iron Quantification.

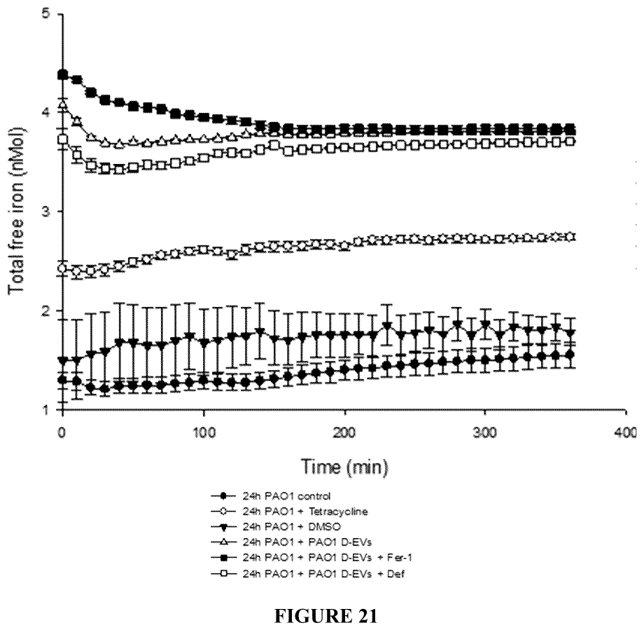

Oxidative stress kills bacteria via biochemical changes in iron pool, reactive oxygen species concentration, and/or glutathione balance. Therefore, to further investigate the mechanism of D-EV treatment on PAO1 biofilms, PAO1 biofilms grown in the presence of D-EVs, D-EVs supplemented with ferroptosis inhibitors (e.g. Ferrostatin-1 (Fer-1)) or deferoxamine (Def.). Fer-1 is a well-known ferroptosis inhibitor in eukaryotic cells, which acts via lipid peroxidation. Def. is an iron chelator, intercalating the ion and reducing its concentration. As exhibited in FIG. 21, there is a significant increase in total free iron in PAO1 biofilms treated with D-EVs. The data presented is also consistent with a model where intracellular iron is increased gradually following bacterial infection. The results support D-EV treatment resulting in physiological changes in the treated biofilms to increase the free iron level and initiate iron-dependent programmed cell death. However, the increase in free iron is not necessarily mechanistically tied to programmed cell death via the Fenton reaction and an increase in ROS.

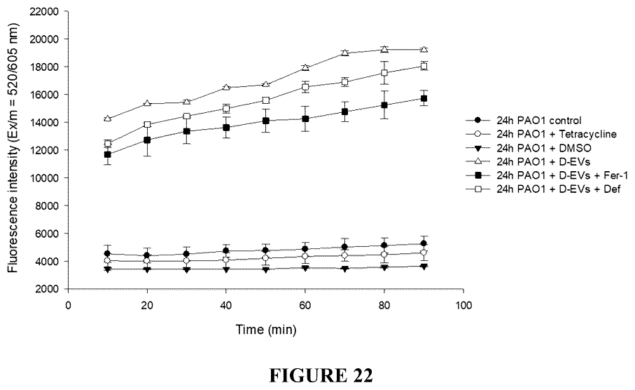

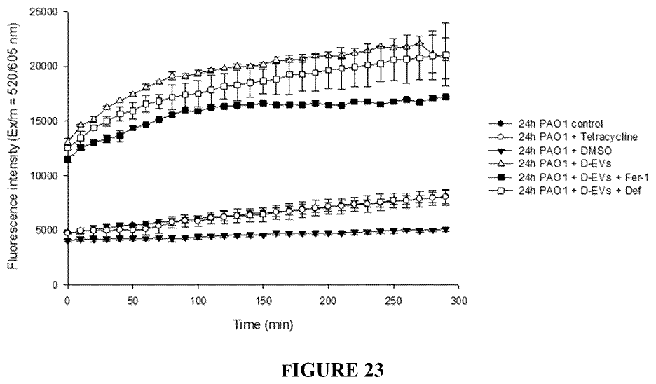

The level of intracellular ROS (e.g. superoxide and hydroxyl radicals) in a culture of PAO1 following various treatments was investigated. The treatments included detection of ROS in PAO1 biofilms. PAO1 cells were incubated with inhibitors for 30 minutes prior to mixing with ROS detection agent for 1 hr. followed by application of D-EVs. Compared to control, the ROS level increased inside the treated biofilms with D-EVs. Additionally, PAO1 biofilms were treated with D-EVs followed by treatment with iron inhibitors (e.g. Fer-1 and Def.). Results shown in FIG. 22 and FIG. 23 indicate that the ROS levels increased in biofilms treated with D-EVs and decreased in biofilms treated with D-EVs supplemented with ferroptosis inhibitors (FIG. 22, FIG. 23).

### Hydrogen Peroxide Measurement

Hydrogen peroxide (H₂O₂) is a non-radical reactive oxygen species that is produced naturally as a byproduct of physiological pathways inside eukaryotes and prokaryotes. H₂O₂ reacts with free ions (e.g., Fe²⁺) to causes oxidative stress. One example of oxidative stress induced by hydrogen peroxide is the Fenton reaction, which produces hydroxyl radicals with concurrent cell damage. Hydrogen peroxide is scavenged through the enzymatic activities of catalase and superoxide dismutase, which convert hydrogen peroxide to water.

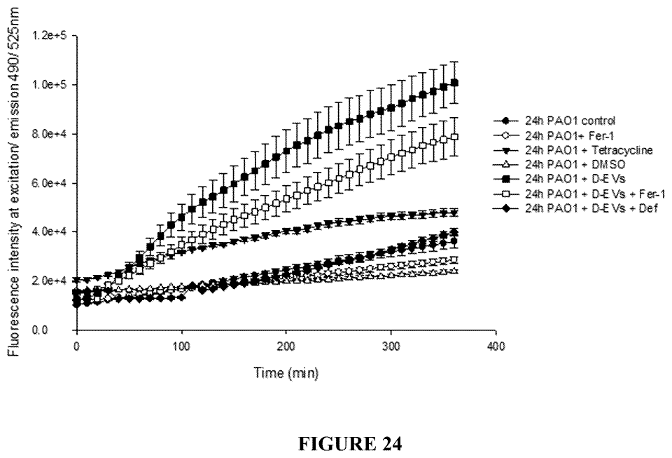

The intracellular concentration of hydrogen peroxide in PAO1 biofilms following treatment with tetracycline, PAO1 D-EVs, and PAO1 D-EVs with two different ferroptosis inhibitors Fer-1 and Def. (FIG. 24). As shown in FIG. 23, the hydrogen peroxide levels increased following treatment of the biofilm with D-EVs, and decreased in biofilms treated with D-EVs followed by subsequent addition of Fer-1 and Def.

### Measurement of Catalase Activity.

Catalase is an antioxidant enzyme that breaks down hydrogen peroxide into water and oxygen to protect cells from oxidative stress. The intracellular concentration of catalase activity in PAO1 biofilms following various treatments was investigated to determine if catalase activity would increase following treatment with D-EVs.

PAO1 biofilms following 24 hr. of growth were treated with tetracycline, PAO1 D-EVs, PAO1 D-EVs with either Fer-1 or Def. PBS and DMSO were included as negative controls. The catalase activity levels increased in biofilms treated with D-EVs. Catalase activity levels decreased in biofilms treated with D-EVs and supplemented with ferroptosis inhibitors (Fer-1 and Def.). Inactivity of catalase increased the pool of H₂O₂ to initiate Fenton reactions resulting in ferroptosis.

Activity of E. coli ΔkatE, ΔkatG, and ΔkatEG

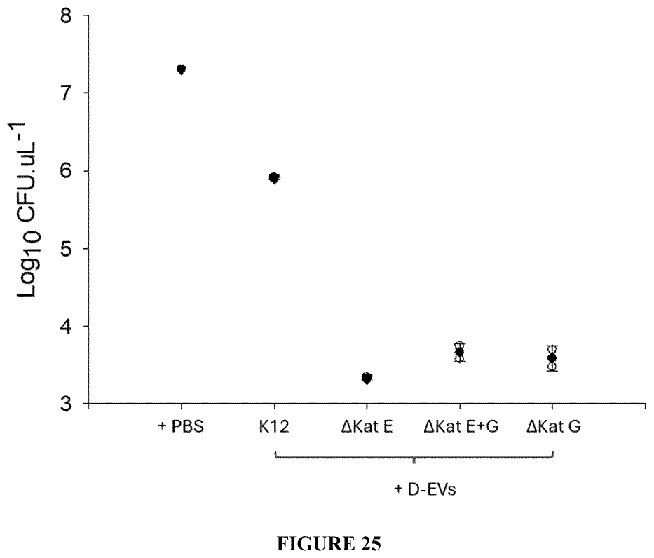

E. coli ΔkatE, ΔkatG, and ΔkatEG mutant biofilms were treated with D-EVs to evaluate the oxidative stress. The E. coli ΔkatE, ΔkatG, and ΔkatEG strains lack catalases and do not exhibit cellular protection against hydrogen peroxide. Following a 24 hr. growth of the three biofilm strains were treated with PAO1 D-EVs and compared to the growth of E. coli K12 biofilm treated with PAO1 D-EVs (FIG. 25).

### Evaluation of Lipid Hydroperoxide (LPO) Following D-EV Treatment

Lipid hydroperoxides indicate oxidative stress that results from high ferrous iron concentration. Lipid hydroperoxides are unstable and react directly with ferrous ions to produce ferric iron. To monitor the concentration of ferric ions, Fe³⁺ are intercalated with thiocyanate to produce a colorimetric readout. The intracellular concentration of lipid hydroperoxide in PAO1 biofilms following various treatments was investigated. 24 hr. P. aeruginosa PAO1 biofilms were treated for 24 hr. with tetracycline, PAO1 D-EVs, PAO1 D-EVs+ferroptosis inhibitors Fer-1 and Def. PBS and DMSO were added as negative controls. Lipid hydroperoxides were extracted in chloroform. FIG. 25 shows lipid hydroperoxide levels in treated and untreated biofilms. Lipid hydroperoxide was quantified in control PAO1 biofilms as 0.1±2.5 nM. Lipid hydroperoxide in PAO1 biofilms treated with D-EVs was found to be 17.1±9.4 nM. The concentration decreased to 7.4±1.04 and 7.9±2.01 nM in PAO1 biofilms treated with D-EVs supplemented with Fer-1 and Def, respectively.

### Evaluation of Redox Balance Following D-EV Treatment

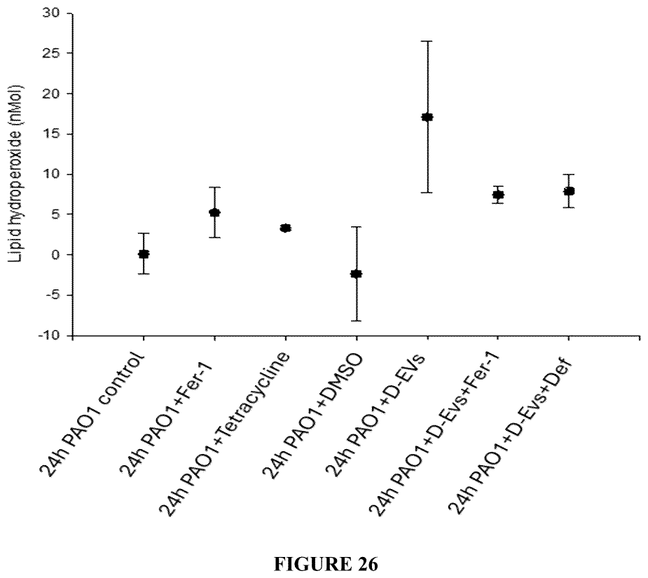

Lactate dehydrogenase catalyzes the conversion between pyruvate and lactate and maintains the redox balance of NADH and NAD⁺. As a result of membrane damage and lipid peroxidation during ferroptosis, LDH levels increase. Therefore, the activity of lactate dehydrogenase in PAO1 biofilms with various treatments was investigated. 24 hr. P. aeruginosa PAO1 biofilms were treated for 24 hr. with tetracycline, PAO1 D-EVs, PAO1 D-EVs with ferroptosis inhibitors Fer-1 and Def. PBS and DMSO were used as negative controls. As demonstrated in FIG. 26, lactate dehydrogenase levels in treated and untreated biofilms were quantified. The activity of lactate dehydrogenase in control PAO1 biofilms was 1.03±0.06 mU/mL. The activity of lactate dehydrogenase in PAO1 biofilms treated with D-EVs was 1.4±0.2 mU/mL. Lactate dehydrogenase activity decreased to 0.4±0.03 and 0.7±0.13 mU/mL in PAO1 biofilms treated with D-EVs and supplemented with Fer-1 and Def, respectively. These results support a model of increased LDH release after ferroptosis induction.

### Production of Superoxide Dismutase

Reactive oxygen species (ROS) are a natural by-product of the normal metabolism of oxygen. The two major sources of cellular ROS are Complex I (NADH dehydrogenase ubiquinone-ubiquinol reductase) and Complex III (ubiquinol cytochrome c reductase), both part of the mitochondrial electron transport chain. These two complexes generate ROS particularly when electron transport is slowed by high mitochondrial membrane potential (Δψm). The major product of ROS in mitochondrial are superoxide and hydroperoxyl radicals. Superoxide is generated in complex Ill in the presence of slow electron transport. The slow electron transport results in formation of a ubisemiquinone anion radical which reacts with oxygen dissolved in the membrane. The exact source of superoxide generated by complex I not as well understood, but it is likely due to electron leakage from its iron-sulfur clusters. Low levels (or optimum levels) of ROS play an important role in signaling pathways. However, when ROS production increases and overwhelms the cellular antioxidant capacity, it can induce macromolecular damage (e.g. reacting with DNA, proteins, and lipids) and disrupt thiol redox circuits. In the first instance, damage can lead to apoptosis, and in the second instance, disruption of thiol redox circuits can lead to aberrant cell signaling and dysfunctional redox control. Therefore, to assess redox balance and quantify superoxide levels, the quantification of superoxide inside biofilms following treatment with D-EVs was investigated. Additionally, to address the role of ferroptosis in redox signaling or formation of superoxide, the biofilms were additionally treated with ferroptosis inhibitors after D-EV treatment.

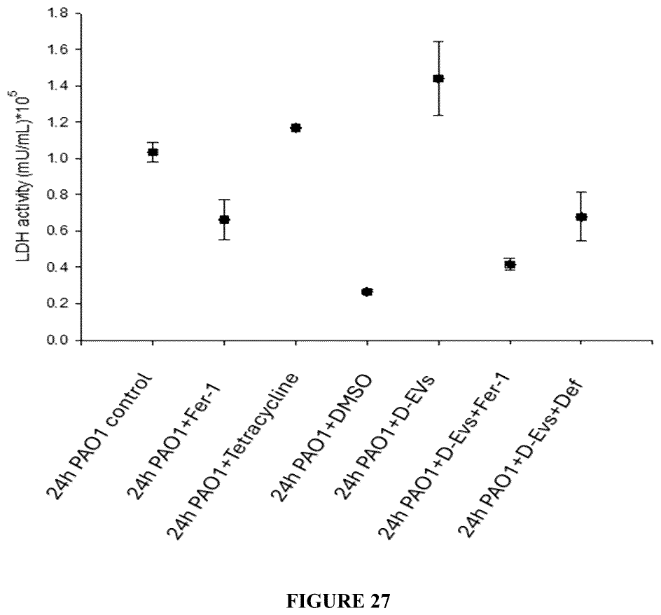

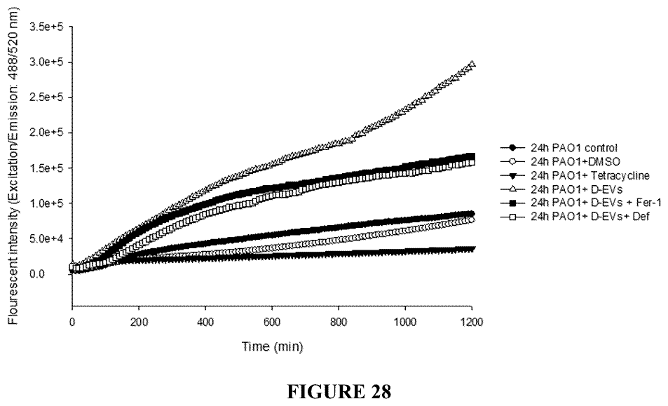

P. aeruginosa PAO1 biofilms were treated for 24 hours with tetracycline, PAO1 D-EVs, PAO1 D-EVs+ferroptosis inhibitors Fer-1 and Def. PBS and DMSO were added to biofilms as negative controls. The superoxide levels were quantified over time. FIG. 27 displays the increase level of superoxide inside PAO1 biofilms treated with D-EVs compared to controls. The concentration of superoxide as measured by fluorescence intensity of a reporter compound indicates when treated with D-EVs, superoxide concentration increased. Additionally, when PAO1 biofilms were treated with D-EVs followed by ferroptosis inhibitors, the concentration of superoxide increased, but not to the same level as seen with D-EVs alone.

1. A composition comprising one or more death extracellular vesicles (D-EVs).

2. The composition of claim 1, wherein the one or more D-EVs comprise one or more proteins selected from the group consisting of a redox cycle-regulating protein, an iron-acquisition protein, a quorum sensing (QS) protein, and any combination thereof.

3. The composition of claim 1, wherein the one or more D-EVs comprise one or more proteins selected from Table 5 and Table 6.

4. The composition of claim 1, wherein the one or more D-EVs is isolated from a microorganism.

5. The composition of claim 1, wherein the one or more D-EVs is isolated from a microorganism under a stress-induced condition.

6. The composition of claim 1, wherein the composition is an antimicrobial composition.

7. A pharmaceutical composition comprising i) one or more death extracellular vesicles (D-EVs) and ii) a pharmaceutically acceptable carrier.

8. The pharmaceutical composition of claim 7, wherein the pharmaceutical composition further comprises an iron chelator.

9. The pharmaceutical composition of claim 7, wherein the pharmaceutical composition further comprises a second therapeutic agent, wherein the second therapeutic agent is an antibiotic.

10. A method of treating an infection in a patient, the method comprising a step of administering a therapeutically effective amount of the pharmaceutical composition of claim 7 to the patient, wherein the pharmaceutical composition provides treatment of the infection.

11. The method of claim 10, wherein the treatment of the infection comprises growth inhibition of the microbe, reduction of growth of a biofilm comprising the microbe, reduction of size of a biofilm comprising the microbe, or any combination thereof.

12. The method of claim 10, wherein the treatment of the infection comprises ROS-based ferroptosis of the microbe.

13. The method of claim 10, wherein the treatment of the infection comprises an increase of ROS accumulation in the microbe, an increase in iron accumulation by the microbe, disruption of iron metabolism of the microbe, or any combination thereof.

14. The method of claim 10, wherein the treatment of the infection comprises disruption of quorum sensing of the microbe.

15. The method of claim 10, wherein the method further comprises administration of an iron chelator to the patient.

16. A method of collecting one or more extracellular vesicles (EVs) from a composition comprising one or more microorganisms, the method comprising:
(a) culturing the one or more microorganisms, wherein the culturing comprises secretion of one or more EVs from the microorganisms;
(b) separating the EVs from the composition; and
(c) collecting the EVs separated in step (b).

17. The method of claim 16, wherein the culturing comprises induction of a stress condition to the one or more microorganisms.

18. The method of claim 17, wherein the stress condition is selected from the group consisting of a temperature stress condition, a nutrient starvation, an oxidative stress, decreasing the pH of the composition to below a pH of 7, increasing the pH of the composition to above a pH of 7, increasing the osmotic pressure of the composition, increasing the salinity of the composition, applying UV stress to the composition, introducing a chemical to the composition, deprivation of oxygen to the composition, or combinations thereof.

19. The method of claim 16, wherein the one or more EVs comprise a death EV (D-EV).

20. The method of claim 16, wherein the collected EVs are subsequently lyophilized or encapsulated.
